SEQUENCE LISTING (35 sequences)

SEQ ID NO: 1 - DNA, 1011 bp, Homo sapiens
agtgttcggctggggcaggcacgctgtggctggctacttcccttcctcccatcccccttgggccaaacgggatcggtgcttctggtgagacgcctccccatgcacatcactcccaggtgccctagggggcacatttcccacaactcccagagggcaggtttctagaaagtgccaccagtggggaggcgccacaacttcactgccattttgtgaggtgccgccgtctctcctccagcaaggaaacaatgaccgataaaacagagaaggtggctgtagatcctgaaactgtgtttaaacgtcccagggaatgtgacagtccttcgtatcagaaaaggcagaggatggccctgttggcaaggaaacaaggagcaggagacagccttattgcaggctctgccatgtccaaagcaaagaagcttatgacaggacatgctattccacccagccaattggattctcagattgatgacttcactggtttcagcaaagataggatgatgcagaaacctggtagcaatgcacctgtgggaggaaacgttaccagcagtttctctggagatgacctagaatgcagagaaacagcctcctctcccaaaagccaacaagaaattaatgctgatataaaacgtaaattagtgaaggaactccgatgcgttggacaaaaatatgaaaaaatcttcgaaatgcttgaaggagtgcaaggacctactgcagtcaggaaacgattttttgaatccatcatcaaggaagcagcaagatgtatgagacgagactttgttaagcaccttaagaagaaactgaaacgtatgatttgagaatacttgtccctggaggattatcacaccccaaatgcataatctcgttaatgattgaggagagaaaaggatcagattgctgttttctacaatggagcaggatattgctgaagtctcctggcatatgttaccgaatcaactggccttccagaggctaagaaatttctgttagtaaaagatgttctttttcccaaaa

SEQ ID NO: 2 - DNA, 856 bp, Homo sapiens
agtgttcggctggggcaggcacgctgtggctggctacttcccttcctcccatcccccttgggccaaacgggatcggtgcttctggaaacaatgaccgataaaacagagaaggtggctgtagatcctgaaactgtgtttaaacgtcccagggaatgtgacagtccttcgtatcagaaaaggcagaggatggccctgttggcaaggaaacaaggagcaggagacagccttattgcaggctctgccatgtccaaagcaaagaagcttatgacaggacatgctattccacccagccaattggattctcagattgatgacttcactggtttcagcaaagataggatgatgcagaaacctggtagcaatgcacctgtgggaggaaacgttaccagcagtttctctggagatgacctagaatgcagagaaacagcctcctctcccaaaagccaacaagaaattaatgctgatataaaacgtaaattagtgaaggaactccgatgcgttggacaaaaatatgaaaaaatcttcgaaatgcttgaaggagtgcaaggacctactgcagtcaggaaacgattttttgaatccatcatcaaggaagcagcaagatgtatgagacgagactttgttaagcaccttaagaagaaactgaaacgtatgatttgagaatacttgtccctggaggattatcacaccccaaatgcataatctcgttaatgattgaggagagaaaaggatcagattgctgttttctacaatggagcaggatattgctgaagtctcctggcatatgttaccgaatcaactggccttccagaggctaagaaatttctgttagtaaaagatgttctttttcccaaaa

SEQ ID NO: 3 - protein, 189 aa, Homo sapiens
MTDKTEKVAVDPETVFKRPRECDSPSYQKRQRMALLARKQGAGDSLIAGSAMSKAKKLMTGHAIPPSQLDSQIDDFTGFSKDRMMQKPGSNAPVGGNVTSSFSGDDLECRETASSPKSQQEINADIKRKLVKELRCVGQKYEKIFEMLEGVQGPTAVRKRFFESIIKEAARCMRRDFVKHLKKKLKRMI

SEQ ID NO: 4 - protein, 121 aa, Homo sapiens
PPSQLDSQIDDFTGFSKDRMMQKPGSNAPVGGNVTSSFSGDDLECRETASSPKSQQEINADIKRKLVKELRCVGQKYEKIFEMLEGVQGPTAVRKRFFESIIKEAARCMRRDFVKHLKKKL

SEQ ID NO: 5 - protein, 64 aa, Homo sapiens
NADIKHQLMKEVRKFGRKYERIFILLEEVQGPLEMKKQFVEFTIKEAARFKRRVLIQYLEKVLE

SEQ ID NO: 6 - protein, 69 aa, Homo sapiens
EINADIKRKLVKELRCVGQKYEKIFEMLEGVQGPTAVRKRFFESIIKEAARCMRRDFVKHLKKKLKRMI

SEQ ID NO: 7 - protein, 450 aa, Homo sapiens
MPILLFLIDTSASMNQRTDLGTSYLDIAKGAVELFLKLRARDPASRGDRYMLVTYDEPPYCIKAGWKENHATFMSELKNLQASGLTTLGQALRSSFDLLNLNRLISGIDNYGQGRNPFFLEPSILITITDGNKLTSTAGVQEELHLPLNSPLPGSELTKEPFRWDQRLFALVLRLPGVASTEPEQLGSVPTDESAITQMCEVTGGRSYCVRTQRMLNQCLESLVQKVQSGVVINFEKTGPDPLPIGEDGLMDSSRPSNSFAAQPWHSCHKLIYVRPNSKTGVPVGHWPIPESFWPDQNLPSLPPRTSHPVVRFSCVDCEPMVIDKLPFDKYELEPSPLTQYILERKSPHTCWQVFVTSSGKYNELGYPFGYLKASTTLTCVNLFVMPYNYPVLLPLLDDLFKVHKLKPNLKWRQAFDSYLKTLPPYYLLVCICVYICINCINDNSCHKKC

SEQ ID NO: 8 - protein, 887 aa, Homo sapiens
MPILLFLIDTSASMNQRSHLGTTYLDTAKGAVETFMKLRARDPASRGDRYMLVTFEEPPYAIKAGWKENHATFMNELKNLQAEGLTTLGQSLRTAFDLLNLNRLVTGIDNYGQGRNPFFLEPAIIITITDGSKLTTTSGVQDELHLPLNSPLPGSELTKEPFRWDQRLFALVLRLPGTMSVESEQLTGVPLDDSAITPMCEVTGGRSYSVCSPRMLNQCLESLVQKVQSGVVINFEKAGPDPSPVEDGQPDISRPFGSQPWHSCHKLIYVRPNPKTGVPIGHWPVPESFWPDQNSPTLPPRTSHPVVKFSCTDCEPMVIDKLPFDKYELEPSPLTQFILERKSPQTCWQVYVSNSAKYSELGHPFGYLKASTALNCVNLFVMPYNYPVLLPLLDDLFKVHKAKPTLKWRQSFESYLKTMPPYYLGPLKKAVRMMGAPNLIADSMEYGLSYSVISYLKKLSQQAKIESDRVIGSVGKKVVQETGIKVRSRSHGLSMAYRKDFQQLLQGISEDVPHRLLDLNMKEYTGFQVALLNKDLKPQTFRNAYDIPRRNLLDHLTRMRSNLLKSTRRFLKGQDEDQVHSVPIAQMGNYQEYLKQVPSPLRELDPDQPRRLHTFGNPFKLDKKGMMIDEADEFVAGPQNKHKRPGEPNMQGIPKRRRCMSPLLRGRQQNPVVNNHIGGKGPPAPTTQAQPDLIKPLPLHKISETTNDSIIHDVVENHVADQLSSDITPNAMDTEFSASSPASLLERPTNHMEALGHDHLGTNDLTVGGFLENHEEPRDKEQCAEENIPASSLNKGKKLMHCRSHEEVNTELKAQIMKEIRKPGRKYERIFTLLKHVQGSLQTRLIFLQNVIKEASRFKKRMLIEQLENFLDEIHRRANQINHINSN

SEQ ID NO: 9 - protein, 904 aa, Homo sapiens
MQASPLQTSQPTPPEELHAAAYVFTNDGQQMRSDEVNLVATGHQSKKKHSRKSKRHSSSKRRKSMSSWLDKQEDAAVTHSICEERINNGQPVADNVLSTAPPWPDATIAHNIREERMENGQSRTDKVLSTAPPQLVHMAAAGIPSMSTRDLHSTVTHNIREERMENGQPQPDNVLSTGPTGLINMAATPIPAMSARDLYATVTHNVCEQKMENVQPAPDNVLLTLRPRRINMTDTGISPMSTRDPYATITYNVPEEKMEKGQPQPDNILSTASTGLINVAGAGTPAISTNGLYSTVPHNVCEEKMENDQPQPNNVLSTVQPVIIYLTATGIPGMNTRDQYATITHNVCEERVVNNQPLPSNALSTVLPGLAYLATADMPAMSTRDQHATIIHNLREEKKDNSQPTPDNVLSAVTPELINLAGAGIPPMSTRDQYATVNHHVHEARMENGQRKQDNVLSNVLSGLINMAGASIPAMSSRDLYATITHSVREEKMESGKPQTDKVISNDAPQLGHMAAGGIPSMSTKDLYATVTQNVHEERMENNQPQPSYDLSTVLPGLTYLTVAGIPAMSTRDQYATVTHNVHEEKIKNGQAASDNVFSTVPPAFINMAATGVSSMSTRDQYAAVTHNIREEKINNSQPAPGNILSTAPPWLRHMAAAGISSTITRDLYVTATHSVHEEKMTNGQQAPDNSLSTVPPGCINLSGAGISCRSTRDLYATVIHDIQEEEMENDQTPPDGFLSNSDSPELINMTGHCMPPNALDSFSHDFTSLSKDELLYKPDSNEFAVGTKNYSVSAGDPPVTVMSSVETVPNTPQISPAMAKKINDDIKYQLMKEVRRFGQNYERIFILLEEVQGSMKVKRQFVEFTIKEAARFKKVVLIQQLEKALKEIDSHCHLRKVKHMRKR

SEQ ID NO: 10 - DNA, 20 bp, Artificial sequence
  THEG forward primer
ccaaaacccaagccacatgt

SEQ ID NO: 11 - DNA, 21 bp, Artificial sequence
  THEG reverse primer
gcacttgtccgactgagcttt

SEQ ID NO: 12 - DNA, 28 bp, Artificial sequence
  THEG probe
cagaccataaccgccctccttcacttgg

SEQ ID NO: 13 - DNA, 23 bp, Artificial sequence
  NALP4 forward primer
ttgtcacctctcacccattgatt

SEQ ID NO: 14 - DNA, 26 bp, Artificial sequence
  NALP4 reverse primer
caggatacattcagatacgtcagctt

SEQ ID NO: 15 - DNA, 29 bp, Artificial sequence
  NALP4 probe
tgaagtccttgctggccttctaaccaaca

SEQ ID NO: 16 - DNA, 24 bp, Artificial sequence
  COXVIB2 forward primer
ccgtaactgctaccagaacttcct

SEQ ID NO: 17 - DNA, 25 bp, Artificial sequence
  COXVIB2 reverse primer
agtggtacacgcggaaatagtactc

SEQ ID NO: 18 - DNA, 26 bp, Artificial sequence
  COXVIB2 probe
actaccaccgctgcctcaagaccagg

SEQ ID NO: 19 - DNA, 25 bp, Artificial sequence
  LOC348120 forward primer
tggattccaattcatctgactacag

SEQ ID NO: 20 - DNA, 21 bp, Artificial sequence
  LOC348120 reverse primer
cttccgcttacctccaactga

SEQ ID NO: 21 - DNA, 30 bp, Artificial sequence
  LOC348120 probe
ctgcaggtgattcatttgcaaggtaagctg

SEQ ID NO: 22 - DNA, 20 bp, Artificial sequence
  CT45 forward primer
ctctgccatgtccaaagcaa

SEQ ID NO: 23 - DNA, 26 bp, Artificial sequence
  CT45 reverse primer
aagtcatcaatctgagaatccaattg

SEQ ID NO: 24 - DNA, 31 bp, Artificial sequence
  CT45 probe
aagcttatgacaggacatgctattccaccca

SEQ ID NO: 25 - protein, 394 aa, Homo sapiens
MATAQLQRTPMSALVFPNKISTEHQSLVLVKRLLAVSVSCITYLRGIFPECAYGTRYLDDLCVKILREDKNCPGSTQLVKWMLGCYDALQKKYLRMVVLAVYTNPEDPQTISECYQFKFKYTNNGPLMDFISKNQSNESSMLSTDTKKASILLIRKIYILMQNLGPLPNDVCLTMKLFYYDEVTPPDYQPPGFKDGDCEGVIFEGEPMYLNVGEVSTPFHIFKVKVTTERERMENIDSTILSPKQIKTPFQKILRDKDVEDEQEHYTSDDLDIETKMEEQEKNPASSELEEPSLVCEEDEIMRSKESPDLSISHSQVEQLVNKTSELDMSESKTRSGKVFQNKMANGNQPVKSSKENRKRSQHESGRIVLHHFDSSSQESVPKRRKFSEPKEHI

SEQ ID NO: 26 - DNA, 3161 bp, Homo sapiens
acgctgcctccatgcctgcctcccctctctgccacgcccccaccacgcgctttgcactctggggcccacgcacttccctgaagagtctagaagctgctcctcatttccagactttccgggcgcccaccaggctcccgggctctcaccggagaccacagggaggagcttcaaggtaccccaagtccctgcggcctccagcagtgaagagttccacaagggagtcttcctacgtcactgaataatgaatgaagatgagaggggaaaagagaagagacaaagtcaatccaaaaagctctcaaaggaaattaaactggattccatcatttcctacctatgattacttcaaccaagtgacgctacagttattagatggctttatgattacactgagcacagatggagtgatcatttgtgtggctgaaaacatctcttctcttcttggacatttaccagctgagattgtgggcaaaaaattattaagccttctgcctgatgaagagaaagatgaagtctaccaaaagattattctcaaatttcctttactaaactcagaaacacatattaaattttgctgtcatttaaaaagaggaaatgtcgaacatggtgatagttctgcttacgaaaacgtgaaatttattgtgaatgtaagagatatttgtaatgagtttcctgtggtctttagtggcttgttttccagccacctctgtgctgactttgctgcatgtgttcctcaggaggatcggctttatcttgtggggaatgtttgcattctcaggactcagctcctgcagcaactttacacttcaaaggcagtcagtgatgaagctgtacttacacaagattcagatgaggaaccttttgtgggagagctcagtagctctcaaggtcaaagaggacacactagcatgaaagccgtgtacgttgaacccgctgctgctgctgctgctgctgctatctcagacgaccaaattgatattgcagaggttgagcagtatggtccacaagaaaacgttcacatgtttgtagattctgattcaacttattgctccagtacagttttcctggatactatgcctgaatctccagccttatccttgcaagactttcgaggtgagcctgaggtgaatccattgtacagggcagacccagtggacctggagttctcggtggatcaggtggactcagtggaccaggagggcccaatggaccagcaggacccagagaacccagttgccccgttggaccaggcaggcctgatggatccagtggatccagaggactcagtggacctgggggctgctggcgcaagtgctcagccattacagccatcatcaccagttgcatatgacatcattagccaggaactggaactgatgaagaagttgaaggagcagctagaagagaggacttggttgctgcatgatgccatccaaaaccagcagaatgcattggaattgatgatggatcaccttcagaagcagccaaacacattacgccacgttgtcattcctgatctccaatcttcggaggcagtgcccaagaaacaacagaaacaacacgctgggcaagtgaagcggcctctcccacatcccaaggacgtcaagtgtttctgtggtttatctttatccaactctctcaaaaacactggggagcttcaggagccttgtgttgccttcaaccagcagcaactggtgcagcaagaacaacacctgaaggagcagcagcggcagctgcgggagcagctgcaacagctgagagagcaaaggaaggtgcagaagcagaagaagatgcaggagaagaagaagctgcaggagcagaaaatgcaggagaagaagaagctgcaggagcagaggcggcaaaagaagaagaagctacaggagcggaagaagtggcaggggcagatgctacagaaagagccagaggaggagcagcagaagcagcagttgcaagagcagccactgaagcataatgtcatcgtggggaatgagagggtgcagatatgcctgcaaaacccacgtgacgtatctgtgcccctctgcaatcaccctgttagatttttacaggcccaacccattgttcctgtccagagagcagctgaacaacagccctctggcttctatcaagatgaaaactgtgggcaacaggaagatgagagtcaaagtttttatcctgaggcgtatcaagggccccccgtgaaccagctgccattgatagatacctcaaactctgaggcaatttcttcttccagcattcctcagtttcccataacttcagactcaaccataagcaccctggagaccccacaggattacatccggctttggcaagagttgtctgattcactcggtcctgttgtccaagtgaacacttggtcttgcgatgagcagggcaccctgcacggccaacccacctaccatcaggtgcaagtttctgaggtaggagtcgagggacctcctgatccacaggctttccaaggccctgctgcataccagccagaccagatgagatctgcggagcagaccagattgatgcctgcagagcaacgtgactcaaataagccgtgctaacagtactttcatgaccagtgatgaggggaaatggggggagggggcaggccaatgaggtctgcatggccaggggaccttcaaggtgcgtaaagtcccttggggtagggtttagtgggtagagacttatttgtttcctgataggttatgtttgtaattgtttgttaagcacagcctgtttcttggaagttatgctgtagaggcagcctgtgatccgtagtatgctagggtttgacagcagccagccacagctggatctgatgtcttgtctgccccgcccagctttgcatatccatgttctaccacaggaaggtggcctgccaagagtctgctcaaagttttcaacataaagaaaaaagaaaaaaaaatgccaaagtgcttttcaatctagtaaatctagagggttgttttgtcttagccacaagaattccgaggtcttgaccctgatgatcaacctgcctcccctccatagtcttgttggagaagcccagagagaatgggactccaactaagggaacctgaaatcaactcaatggaggcacttcagagctaaaataattatggctaccttgcttaataaacattttcgttcactgc

SEQ ID NO: 27 - protein, 394 aa, Homo sapiens; identical to SEQ ID NO: 25

SEQ ID NO: 28 - protein, 78 aa, Homo sapiens
TELKAQIMKEIRKPGRKYERIFTLLKHVQGSLQTRLIFLQNVIKEASRFKKRMLIEQLENFLDEIHRRANQINHINSN

SEQ ID NO: 29 - protein, 25 aa, Homo sapiens
QQLEKALKEIDSHCHLRKVKHMRKR

SEQ ID NO: 30 - DNA, 1831 bp, Homo sapiens
atgcgcgtcgggcacagcgcgtgcagcctcgtgcagctcttctggtctccggcgcccgcccctcagacgtaatgttgaattaaagaaaatactttatcagaagaagatggccactgcccagttgcagaggactcccatgagtgcactggtatttcccaataagatatcaactgaacaccagtctttggtgttagtgaagaggcttctagcagtttcagtatcctgtatcacgtatttgaggggaatattcccagaatgcgcttatggaacaagatatctagatggatgctaggatgttatgatgctttacagaaaaaatatctaaggatggttgttctagctgtatacacaaacccagaagatcctcagacaatttcagaatgttaccaattcaaattcaaatacaccaataatggaccactcatggacttcataagtaaaaaccaaagcaacgaatctagcatgttgtctactgacaccaagaaagcaagcattctcctcattcgcaagatttatatcctaatgcaaaatctggggcctttacctaatgatgtttgtttgaccatgaaacttttttactatgatgaagttacacccccagattaccagcctcccggttttaaggatggtgattgtgaaggagttatatttgaaggggaacctatgtatttaaatgtgggagaagtctcaacaccttttcacatcttcaaagtaaaagtgaccactgagagagaacgaatggaaaatattgactcaactatactatcaccaaaacaaataaaaacaccatttcaaaaaatcctgagggacaaagatgtagaagatgaacaggagcattatacaagtgatgatttggacattgaaactaaaatggaagaacaggaaaaaaaccctgcatcttctgaacttgaagaaccaagtttagtttgtgaggaagatgaaattatgaggtctaaagaaagtccagatctttctatttctcattctcaggttgagcagttagtcaataaaacatctgaacttgatatgtctgaaagcaaaacaagaagtggaaaagtctttcagaataaaatggcaaatggaaatcaaccagtaaaatcttccaaagaaaatcggaagagaagtcaacatgaatctgggagaatagtcctccatcactttgattcttctagtcaagagtcagtgccaaaaaggagaaagtttagtgaaccaaaggaacatatataaaaattatttttgttctgcaggcttgcagagttcttctcaccatttaaactgaaggaccctatattatatttccctaactctgaagatgtatatgtagtttaaagcagtttgtacactaaaactaagtttttggctgactgtcatattgtggtccttaatcttgagataaatccaatagaacttttgaataaaagcaaaagtacaaatgtcataattgattcggtaataagtaaaatttcaaaattgattttgttcattacctacttaatatttcctttaaatatatactaactgttaaggccctctaatgccatttttctaaacagtaatgtttactttggtattaaaatttggtatggattcactttttacttatgttaaaattataccatttaactggctcttttgtcattgtgctgttattaaaacaatgttcttcaatattttgacataatgtattaacattttaatatataatgtacaatttaagaattggtgctttacctttactatgcttttttttacaggacaaaaagactgatttttaaagtatggcattttttgcagcataaataaaatattgttcagcaaaaaaaaaaaaaaa

SEQ ID NO: 31 - protein, 61 aa, Homo sapiens
MATAQLQRTPMSALVFPNKISTEHQSLVLVKRLLAVSVSCITYLRGIFPECAYGTRYLDGC

SEQ ID NO: 32 - protein, 323 aa, Homo sapiens
MRLWNKISRWMLGCYDALQKKYLRMVVLAVYTNPEDPQTISECYQFKFKYTNNGPLMDFISKNQSNESSMLSTDTKKASILLIRKIYILMQNLGPLPNDVCLTMKLFYYDEVTPPDYQPPGFKDGDCEGVIFEGEPMYLNVGEVSTPFHIFKVKVTTERERMENIDSTILSPKQIKTPFQKILRDKDVEDEQEHYTSDDLDIETKMEEQEKNPASSELEEPSLVCEEDEIMRSKESPDLSISHSQVEQLVNKTSELDMSESKTRSGKVFQNKMANGNQPVKSSKENRKRSQHESGRIVLHHFDSSSQESVPKRRKFSEPKEHI

SEQ ID NO: 33 - DNA, 1914 bp, Homo sapiens
  CDS (71)..(994)
agcggggtgggagggaagcgtcgacgagctgaggttggacttgttgaaataatcctgatacattcctacaatggccactgctcagctttctcactgcatcacaatacacaaggcttctaaggaaacagttttcccatcccaaatcactaatgagcatgagtcattgaaaatggtgaagaaactttttgctacttccatctcatgtataacatacctaaggggcctgtttccagagagctcttatggagaacgccatttggatgacctcagtttaaaaatcctccgagaagataaaaaatgtcccgggtcactgcatattatcagatggattcaaggttgttttgatgctttggaaaagagatacctacgtatggcagtactgacactttacacagatcccatgggatctgagaaggtgactgagatgtaccagttcaaattcaaatacacgaaagaaggagccactatggattttgacagtcatagcagcagtacaagctttgaaagtggaacaaacaatgaagatattaagaaagccagtgttctactgatccgtaaattgtatatactgatgcaggaccttgagccacttcctaataatgttgtacttactatgaaactccactactataatgcagtgaccccacatgattaccaacccctcggttttaaagaaggggtaaattcacacttcctgctgtttgacaaggagcctatcaacgtgcaagtgggatttgtctccactggctttcatagcatgaaagtaaaagtcatgacagaggctacaaaagtgattgatttggagaacaatctgtttcgggagaacagcactactgagatcgcccatcagggtctagactgtgatgaggaagaagaatgcaatgaccatattcaaagaatgaattttgtgtgcagtcagcaaagttctgagtgctccaggaagaagaggaaggtcagtgaaccagtgaaggtcttcatccctaacagaaaatgaaagctaaatattccttctacatttattttaaaataagtttattttgtaaaaacatgcataaactgtcttagcaggaaagtacattcctgttaccaaaacctttttctaaattttttgctcaattttttttgtcagttgctaagtgctaaattactggccaggtaggacgcgagtccactttgccataaggaaagcgggtcaatagggctgcctctggatagcattacttcaaagctgggttagagatgaggcaccttttatattatatttgtaaaagaaccacagcagctattttggaaagaagctgttgttctcaaaggaaagaaggatttttttgcataattaaactcttcagctatttagactactgtgaaatgttttgacttgttgtaacctgtatactggaatccctctctagctgtattaaaaattcccacagagttgcataggatgtgtggaggtgcttatccaggtgtgatttatgtgttaacctcagtcacgaggttgatcagctaattgtgggaccaaaatgtgccctgcctcaactctggccccaggccctgttctgttatccaatccgaaacacggaacatgctgcaattcaggagtctggctctcttcttaagacctatattaaatatttcccaagaagcacttcagtttatcagtctgtaacctgcaaatgatgctattagttccctccttattctggaagaatttggatagacttaattgatgattagtatgagatgaacaatagatgacttaaagtgttatttctattatttatataacatgagatttagtaacttataaaatgaggtttagtaatttaatttaaatggataggaccaataagttaaatattaagtgtgtgacttatggttaaataaaatgaaaatacaaaaaaaaaaaaaaaa

SEQ ID NO: 34 - protein, 307 aa, Homo sapiens
MATAQLSHCITIHKASKETVFPSQITNEHESLKMVKKLFATSISCITYLRGLFPESSYGERHLDDLSLKILREDKKCPGSLHIIRWIQGCFDALEKRYLRMAVLTLYTDPMGSEKVTEMYQFKFKYTKEGATMDFDSHSSSTSFESGTNNEDIKKASVLLIRKLYILMQDLEPLPNNVVLTMKLHYYNAVTPHDYQPLGFKEGVNSHFLLFDKEPINVQVGFVSTGFHSMKVKVMTEATKVIDLENNLFRENSTTEIAHQGLDCDEEEECNDHIQRMNFVCSQQSSECSRKKRKVSEPVKVFIPNRK

SEQ ID NO: 35 - protein, 324 aa, Homo sapiens
TLPNGLENEKQSLEFMTRLLYVAISTILRERGIFPEEYFKDRYVDGNLLVMTLLRRQDAPEGRLVSWLEKGVHDAIRQKLLKKLSLVITESEDPEDIEVYIFSFVYDEEGSVSARINYGINGQSSKAFELSQLSMDDTRRQFAKLIRKLHICTQLLEPLPQGLILSMRLYYTERVPPDYQPEGFKDSTRAFYTLPVNPEQINIGAVSTPHHKGFVKVLSDATDSMEKAERTDKISDDPFDLILVQQELNKSEEADKSFSQEKTTSITPNVLGNPLVPVDQSEEDLLKSQDSPGTGRCSCECGLDVSKQASVPKTRKSCRKTEHG

(19) United States
(12) Patent Application Publication
(10) Pub. No.: US 2011/0177079 A1
(43) Pub. Date: Jul. 21, 2011

(54) Cancer-testis antigens

(75) Inventors: Yao-Tseng Chen, New York, NY (US); Lloyd J. Old, New York, NY (US); Andrew J.G. Simpson, New York, NY (US); Victor C. Jongeneel, Lausanne (CH); Matthew J. Scanlan, (US); Cynthia H. Scanlan, (US)

(73) Assignees: Ludwig Institute for Cancer Research, New York, NY (US); Cornell Research Foundation, Inc., Ithaca, NY (US)

(21) Appl. No.: 11/662,322

(22) PCT Filed: Sep. 8, 2005
(86) PCT No.: PCT/US05/31770

§ 371 Date: Aug. 28, 2009

Related U.S. Application Data
(60) Provisional application No. 60/607,821, filed on Sep. 8, 2004.
(60) Provisional application No. 60/664,791, filed on Mar. 24, 2005.

(51) Int. Cl. A61K 39/395; A61K 39/00; C07K 14/435; G01N 33/574; C12N 15/12; A61P 35/00

(52) U.S. Cl. 424/139.1; 424/185.1; 424/178.1; 530/350; 530/324; 436/64; 436/501; 536/23.5

(57) ABSTRACT

The invention relates to cancer-testis antigens and the nucleic acid molecules that encode them. The invention further relates to the use of the nucleic acid molecules, polypeptides and fragments thereof in methods and compositions for the diagnosis and treatment of diseases, such as cancer. More specifically, the invention relates to the discovery of novel cancer-testis (CT) antigens.

### RELATED APPLICATIONS

This application claims the benefit under 35 U.S.C. §119(e) of U.S. provisional patent application Ser. No. 60/607,821, filed Sep. 8, 2004, and U.S. provisional patent application Ser. No. 60/664,791, filed Mar. 24, 2005, the contents of which are incorporated herein by reference in their entirety.

### FIELD OF THE INVENTION

The invention relates to cancer-testis antigens and the nucleic acid molecules that encode them. The invention further relates to the use of the nucleic acid molecules, polypeptides and fragments thereof in methods and compositions for the diagnosis and treatment of diseases, such as cancer. More specifically, the invention relates to the discovery of novel cancer/testis (CT) antigens.

### BACKGROUND OF THE INVENTION

Massively parallel signature sequencing (MPSS) is a recent developed technique that can generate from a given cell line or tissue sample millions of signature tags proximal to the 3′ end of the transcripts (Jongeneel, V. C. et al., 2003, Proc Natl Acad Sci USA, 100(8):4702-4705). As there are estimated 200,000 to 300,000 different transcript species per cell, this methodology allows a truly redundant coverage of all different mRNA species expressed in that particular cell line or tissue analyzed. Most of these MPSS tags can be traced back to their corresponding genes, with the number of tags represented by each gene roughly reflecting the mRNA abundance level of that gene in the cell population analyzed.

By comparing MPSS data sets from different tissues, it is then conceivable to identify genes that are expressed in one tissue but not in others, and MPSS thus appears to be an ideal tool to define genes with tissue-restricted expression. Another valuable method for analyzing tissue-specific expression is analyzing expressed sequence tags (ESTs) in EST databases for genes with testis-predominant expression, followed by investigation of their expression in tumors by RT-PCR analysis.

An interesting group of genes with such tissue-specific expression pattern are the cancer-testis (CT) genes, i.e., genes that encode CT antigens. CT antigens are protein antigens that are normally expressed only in germ cells, notably in testis, but are found to be activated and expressed in various cancer cells, presumably as a result of gene de-repression secondary to hypomethylation. These genes are of particular interests to tumor immunologists, as many CT antigens have been shown to be immunogenic in human patients, and are thus considered prime targets for cancer vaccines.

A need therefore exists for identifying additional cancer antigens, in particular cancer-testis antigens. A method that allows detection of cancer genes expressed in a specific tissue would provide a valuable tool for identifying genes that are useful in diagnosing and treating cancer. The ability to identify cancer-testis genes and to determine their expression level in a single or several tissues provides an important tool for selecting genes that are useful in diagnosing and treating cancer.

### SUMMARY OF THE INVENTION

The technique of massively parallel signature sequencing (MPSS) has been used to identify cancer genes in several tissues. In addition, EST database were analyzed for genes with testis-predominant expression, followed by investigation of their expression in tumors by RT-PCR analysis. Several cancer-testis (CT) antigens and cancer testis-like antigens have been identified using these methods. The invention provides, inter alia, isolated nucleic acid molecules, expression vectors containing those molecules and host cells transfected with those molecules. The invention also provides isolated proteins and peptides, antibodies to those proteins and peptides and CTLs which recognize the proteins and peptides. Fragments including functional fragments and variants of the foregoing also are provided. Kits containing the foregoing molecules additionally are provided. The foregoing can be used in the diagnosis, monitoring, research, or treatment of conditions characterized by the expression of one or more cancer-testis or cancer-testis-like antigens.

The invention involves the discovery that these techniques can be used to identify cancer-testis and cancer testis-like genes that are expressed in certain tissues and not others, i.e. tissue-specific genes. The invention provides methods for diagnosing new cancer antigens. Methods are also provided that allow the ability to determine the expression level of the identified cancer-testis genes in a single or several tissues.

According to one aspect of the invention, methods of diagnosing cancer in a subject are provided. The methods include determining the presence or amount of a nucleic acid molecule that encodes an amino acid sequence set forth as SEQ ID NO:3 or a fragment thereof, in a biological sample isolated from the subject, wherein the presence or amount of the nucleic acid molecule in the biological sample indicates the presence of cancer in the subject.

In some embodiments, the nucleic acid molecule comprises the coding sequence of the nucleotide sequence set forth as SEQ ID NOs:1 or 2, or a nucleotide sequence at least about 90% identical to the coding sequence of the nucleotide sequence set forth as SEQ ID NOs:1 or 2. Preferably, the nucleic acid molecule comprises the coding sequence of the nucleotide sequence set forth as SEQ ID NOs:1 or 2 or the nucleotide sequence set forth as SEQ ID NOs:1 or 2.

In other embodiments, the fragment of the polypeptide sequence set forth as SEQ ID NO:3 comprises SEQ ID NO:4 or SEQ ID NO:6.

In still other embodiments, the step of determining the presence or amount of the nucleic acid molecule comprises contacting the biological sample with an agent that selectively binds to the nucleic acid molecule. Preferably, the agent that selectively binds is another nucleic acid molecule. In certain embodiments, the step of determining the presence or amount of the nucleic acid molecule comprises nucleic acid hybridization or nucleic acid amplification. Preferably the nucleic acid amplification is PCR, or the nucleic acid hybridization is performed using a nucleic acid microarray. Preferred primers used in the methods are SEQ ID NO:22 and/or SEQ ID NO:23. Preferably cDNA is detected.

In still other embodiments, the biological sample is tissue, cells and/or blood; the biological sample preferably does not contain testis tissue. In further embodiments, the presence or amount of the nucleic acid molecule in the biological sample is compared with the presence or amount of the nucleic acid molecule in a biological sample from a subject not having cancer.

According to another aspect of the invention, methods of diagnosing cancer in a subject are provided. The methods include determining the presence or amount of a CT45 polypeptide molecule comprising an amino acid sequence set forth as SEQ ID NO:3 or a fragment thereof, in a biological sample isolated from the subject, wherein the presence or amount of the CT45 polypeptide molecule in the biological sample indicates the presence of cancer in the subject. In certain embodiments, the biological sample is contacted with an agent that specifically binds the CT45 polypeptide or fragment thereof. In preferred embodiments, the fragment of the CT45 polypeptide molecule comprises the amino acid sequence set forth as SEQ ID NO:4 or SEQ ID NO:6.

In other embodiments, the agent that selectively binds is an antibody or antigen-binding fragment thereof. Preferably the antibody or fragment thereof is a monoclonal antibody; a chimeric, human, or humanized antibody; a single chain antibody; or a F(ab′)2, Fab, Fd, or Fv fragment. Preferably the antibody or antigen-binding fragment is labeled with a detectable label. Preferred detectable labels include a fluorescent molecule, a radioactive molecule, an enzyme, a metal, a biotin molecule, a chemiluminescent molecule, a bioluminescent molecule, or a chromophore molecule.

In still other embodiments, the biological sample is tissue, cells and/or blood; the biological sample preferably does not contain testis tissue. In further embodiments, the presence or amount of the CT45 polypeptide molecule in the biological sample is compared with the presence or amount of the CT45 polypeptide molecule in a biological sample from a subject not having cancer.

According to a further aspect of the invention, methods for diagnosing cancer in a subject are provided. The methods include determining the presence or amount of antibodies that specifically bind to a CT45 polypeptide molecule comprising an amino acid sequence set forth as SEQ ID NO:3 or a fragment thereof, in a biological sample isolated from the subject, wherein the presence or amount of the antibodies in the biological sample indicates the presence of cancer in the subject. In certain embodiments, the step of determining the presence or amount of antibodies comprises contacting the biological sample with CT45 polypeptide molecules comprising an amino acid sequence set forth as SEQ ID NO:3 or a fragment thereof, and determining the specific binding of the CT 45 polypeptide molecules to the antibodies. Preferably the CT45 polypeptide molecules are bound to a substrate and/or include a detectable label. Preferred detectable labels include a fluorescent molecule, a radioactive molecule, an enzyme, a metal, a biotin molecule, a chemiluminescent molecule, a bioluminescent molecule, or a chromophore molecule. In preferred embodiments, the fragment of the CT45 polypeptide molecule comprises the amino acid sequence set forth as SEQ ID NO:4 or SEQ ID NO:6.

In some embodiments, the methods further include contacting the biological sample with a detectable second antibody that binds the CT45 polypeptide molecules. In still other embodiments, the biological sample is tissue, cells and/or blood; the biological sample preferably does not contain testis tissue.

According to a further aspect of the invention, methods for treating a subject are provided. The methods include administering to a subject having or suspected of having cancer an effective amount of an antibody or antigen-binding fragment thereof that specifically binds to a CT45 polypeptide molecule that comprises an amino acid sequence as set forth in SEQ ID NO:3, or an immunogenic fragment thereof that preferably is eight or more amino acids in length. In certain embodiments, the antibody or a fragment thereof is a monoclonal antibody; a chimeric, human, or humanized antibody; a single chain antibody; a (single) domain antibody or other intracellular antibody; or a F(ab′)₂, Fab, Fd, or Fv fragment. In preferred embodiments, the fragment of the CT45 polypeptide molecule comprises the amino acid sequence set forth as SEQ II) NO:4 or SEQ ID NO:6.

In some embodiments, the antibody or antigen-binding fragment is bound to a cytotoxic agent. Preferably the cytotoxic agent is calicheamicin, esperamicin, methotrexate, doxorubicin, melphalan, chlorambucil, ARA-C, vindesine, mitomycin C, cisplatinum, etopside, bleomycin and/or 5-fluorouracil. Other preferred cytotoxic agents include radioisotopes, including those that emit α radiation, β radiation or γ radiation. Preferred radioisotopes include: ²²⁵Ac, ²¹¹At, ²¹²Bi, ²¹³Bi, ¹⁸⁶Rh, ¹⁸⁸Rh, ¹⁷⁷Lu, ⁹⁰Y, ¹³¹I, ⁶⁷Cu, ¹²⁵I, ¹²³I, ⁷⁷Br, ¹⁵³Sm, ¹⁶⁶Bo, ⁶⁴Cu, ²¹²Pb, ²²⁴Ra and/or ²²³Ra.

According to still another aspect of the invention, methods of inducing an immune response in a subject are provided. The methods include administering to a subject in need of such treatment an isolated polypeptide comprising an amino acid sequence, wherein the amino acid sequence is SEQ ID NO:3, SEQ ID NO:4, SEQ ID NO:6 or an immunogenic fragment thereof, in an amount effective to induce an immune response in the subject. The immunogenic fragment preferably is eight or more amino acids in length. In some embodiments, the subject has or is suspected of having cancer, although prophylactic induction of an immune response also is contemplated. In preferred embodiments, the cancer is melanoma, small cell lung cancer, non-small cell lung cancer, colon cancer, sarcoma or bladder cancer.

In certain embodiments, the immune response includes antibodies that bind to the isolated polypeptide and/or T cells that recognize epitopes of the isolated polypeptide presented by MHC molecules.

The method also can include administering to a subject an antigen presenting cell. Preferred antigen presenting cells are dendritic cells or autologous cells. The dendritic cells can be autologous cells.

In a further aspect of the invention, compositions are provided that include an isolated CT45 polypeptide comprising an amino acid sequence, wherein the amino acid sequence is SEQ ID NO:3, SEQ ID NO:4, SEQ ID NO:6, or an immunogenic fragment thereof. The compositions optionally include a pharmaceutically acceptable carrier, and/or an antigen presenting cell. In preferred embodiments, the antigen presenting cells are dendritic cells or autologous cells. The dendritic cells can be autologous cells.

In preferred embodiments, the compositions include an amount of the isolated polypeptide effective to induce an immune response, or an amount of the isolated polypeptide effective to induce treat cancer.

According to a further aspect of the invention, methods of diagnosing cancer in a subject are provided. The methods include determining the presence or amount of a CT46 nucleic acid molecule that encodes an amino acid sequence set forth as SEQ ID NO:25 or a fragment thereof, in a biological sample isolated from the subject. The presence or amount of the nucleic acid molecule in the biological sample indicates the presence of cancer in the subject. In certain embodiments, the nucleic acid molecule comprises the coding sequence of the nucleotide sequence set forth as SEQ ID NO:26, or a nucleotide sequence at least about 90% identical to the coding sequence of the nucleotide sequence set forth as SEQ ID NO:26. In some preferred embodiments, the nucleic acid molecule includes or consists of the coding sequence of the nucleotide sequence set forth as SEQ ID NO:26 or the nucleotide sequence set forth as SEQ ID NO:26.

In certain embodiments, determining the presence or amount of the nucleic acid molecule includes contacting the biological sample with an agent that selectively binds to the nucleic acid molecule. The agents that selectively binds can be another nucleic acid molecule. Preferably determining the presence or amount of the nucleic acid molecule includes nucleic acid hybridization or nucleic acid amplification. A preferred method of nucleic acid amplification is PCR, and in a preferred method the nucleic acid hybridization is performed using a nucleic acid microarray. Preferably cDNA is detected.

In some embodiments, the biological sample is tissue, cells and/or blood; the biological sample preferably does not contain testis tissue. In still other embodiments, the presence or amount of the nucleic acid molecule in the biological sample is compared with the presence or amount of the nucleic acid molecule in a biological sample from a subject not having cancer.

According to another aspect of the invention, methods of diagnosing cancer in a subject are provided. The methods include determining the presence or amount of a CT46 polypeptide molecule that includes an amino acid sequence set forth as SEQ ID NO:25, SEQ ID NO:31 or SEQ ID NO:32, or a fragment thereof, in a biological sample isolated from the subject. The presence or amount of the CT46 polypeptide molecule in the biological sample indicates the presence of cancer in the subject. In preferred embodiments, the CT46 polypeptide molecule consists of an amino acid sequence set forth as SEQ ID NO:25, SEQ ID NO:31 or SEQ ID NO:32. In certain embodiments, the presence or amount of the CT46 polypeptide molecule in the biological sample is compared with the presence or amount of the CT46 polypeptide molecule in a biological sample from a subject not having cancer. Samples with which the method is performed include tissues, cells and blood.

The biological sample is contacted in some embodiments with an agent that selectively binds the CT46 polypeptide or fragment thereof, which preferably is an antibody or antigen-binding fragment thereof. More preferably, the antibody is a monoclonal antibody, particularly a chimeric, human, humanized or single chain antibody. Preferred antigen-binding fragments include F(ab′)2, Fab, Fd, and Fv fragments.

In certain embodiments, the antibody or antigen-binding fragment is labeled with a detectable label. Preferred detectable labels include a fluorescent molecule, a radioactive molecule, an enzyme, a metal, a biotin molecule, a chemiluminescent molecule, a bioluminescent molecule, and a chromophore molecule.

According to still another aspect of the invention, methods for diagnosing cancer in a subject are provided. The methods include determining the presence or amount of antibodies that specifically bind to a CT46 polypeptide molecule comprising an amino acid sequence set forth as SEQ ID NO:25, SEQ ID NO:31 or SEQ ID NO:32, or a fragment thereof, in a biological sample isolated from the subject. The presence or amount of the antibodies in the biological sample indicates the presence of cancer in the subject. The methods also can include contacting the biological sample with a detectable second antibody that binds the CT46 polypeptide molecules.

In some embodiments, determining the presence or amount of antibodies includes contacting the biological sample with CT46 polypeptide molecule comprising an amino acid sequence set forth as SEQ ID NO:25, SEQ ID NO:31 or SEQ ID NO:32, or a fragment thereof, and determining the specific binding of the CT46 polypeptide molecules to the antibodies. The CT46 polypeptide molecules optionally are bound to a substrate, and optionally include a detectable label. Preferred detectable labels include a fluorescent molecule, a radioactive molecule, an enzyme, a metal, a biotin molecule, a chemiluminescent molecule, a bioluminescent molecule and a chromophore molecule.

In some embodiments, the methods further include contacting the biological sample with a detectable second antibody that binds the CT46 polypeptide molecules. In still other embodiments, the biological sample is tissue, cells and/or blood; the biological sample preferably does not contain testis tissue.

Methods for treating a subject also are provided in a further aspect of the invention. The methods include administering to a subject having or suspected of having cancer an effective amount of an antibody or antigen-binding fragment thereof that specifically binds to a CT46 polypeptide molecule comprising an amino acid sequence set forth as SEQ ID NO:25, SEQ ID NO:31 or SEQ ID NO:32, or an immunogenic fragment thereof that preferably is eight or more amino acids in length.

In certain embodiments, the antibody or a fragment thereof is a monoclonal antibody; a chimeric, human, or humanized antibody; a single chain antibody; a (single) domain antibody or other intracellular antibody; or a F(ab′)₂, Fab, Fd, or Fv fragment.

The antibody or antigen-binding fragment thereof optionally is bound to a cytotoxic agent, preferably calicheamicin, esperamicin, methotrexate, doxorubicin, melphalan, chlorambucil, ARA-C, vindesine, mitomycin C, cisplatinum, etopside, bleomycin, 5-fluorouracil, or a radioisotope. Preferred radioisotopes emit α radiation, β radiation, γ radiation or a combination thereof. Preferred radioisotopes include: ²²⁵Ac, ²¹¹At, ²¹²Bi, ²¹³Bi, ¹⁸⁶Rh, ¹⁸⁸Rh, ¹⁷⁷Lu, ⁹⁰Y, ¹³¹I, ⁶⁷Cu, ¹²⁵I, ¹²³I, ⁷⁷Br, ¹⁵³Sm, ¹⁶⁶Bo, ⁶⁴Cu, ²¹²Pb, ²²⁴Ra and ²²³Ra.

Also provided in another aspect of the invention are methods of inducing an immune response in a subject. The methods include administering to a subject in need of such treatment an isolated CT46 polypeptide molecule comprising an amino acid sequence set forth as SEQ ID NO:25, SEQ ID NO:31, SEQ ID NO:32, or an immunogenic fragment thereof, in an amount effective to induce an immune response in the subject. The immunogenic fragment preferably is eight or more amino acids in length. The subject preferably has or is suspected of having cancer, although prophylactic induction of an immune response also is contemplated. In some embodiments, the cancer is melanoma, small cell lung cancer, non-small cell lung cancer, colon cancer, bladder cancer, breast cancer, esophageal cancer, or endometrial cancer.

In certain embodiments, the immune response comprises antibodies that bind to the isolated polypeptide, while in other embodiments, the immune response comprises T cells that recognize epitopes of the isolated polypeptide presented by MHC molecules.

In further embodiments, the methods include administering an antigen presenting cell, preferably a dendritic cell or an autologous cell.

According to other aspects of the invention, nucleic acid molecules are provided that encode the amino acid sequence of SEQ ID NO:31 or SEQ ID NO:32. Preferably the nucleic acid molecule comprises CT46 transcript variant 2 (SEQ ID NO:30).

The invention in other aspects provides isolated polypeptides that include the amino acid sequences encoded by CT 46 transcript variant 2, including SEQ ID NO:31 and/or SEQ ID NO:32. Compositions that include these polypeptides, polypeptides that include SEQ ID NO:25, or immunogenic fragments of any of these also are provided, which compositions include pharmaceutically acceptable carrier(s) and/or antigen presenting cell(s). The antigen presenting cells preferably are dendritic cells, and may be autologous cells.

In preferred embodiments, the compositions include an amount of the isolated polypeptide effective to induce an immune response, or an amount of the isolated polypeptide effective to induce treat cancer.

The use of the foregoing compositions in the preparation of medicaments for treatment of disease, particularly cancer, also is provided in accordance with the invention.

These and other aspects of the invention, as well as various embodiments thereof, will become more apparent in reference to the drawings and detailed description of the invention.

### BRIEF DESCRIPTION OF THE DRAWINGS

FIG. 1 is a schematic summary of genes analyzed in each step of this study. Gene numbers in each category are shown in parentheses.

FIG. 2 shows expression of 5 CT antigen genes in lung and breast cancer. mRNA expression of the CT genes in non-small cell lung cancer (X) and in breast cancer (O) was examined by real-time RT-PCR. Each symbol represents one case. The dashed line at 100% indicates the testicular level of expression with which tumor expression levels were compared.

FIG. 3 depicts the CT45 gene family and transcript variants. The location of the six copies of CT45 genes and their relation to LOC203522 and SAGE genes on the X chromosome are shown, and the transcriptional orientations are indicated by arrows. The three transcriptional variants of CT45 are shown schematically, with boxes indicating exons. Untranslated regions (UT) at the 5′ and 3′ ends are shown as shaded boxes. Translational initiation sites (ATG) and termination codons (*) are indicated.

FIG. 4 is a multiple sequence alignment of the conserved region between SAGE (SEQ ID NO:29), DDX26 (SEQ ID NO:28), LOC203522 (SEQ ID NO:5) and CT45 (SEQ ID NO:6). Identical sequences are shown in black, whereas conservative changes are shown in gray. The amino acid number for each gene in the starting point of this conserved segment is indicated.

FIG. 5 is an analysis flowchart of 20 CT candidate genes. Gene expression in normal tissues and cancer cell lines was evaluated by qualitative RT-PCR. Ubiquitous expression corresponded to the presence of PCR products of similar intensity in most or all normal tissues examined, as judged by the ethidium bromide staining on agarose gels. Variable expression means significant expression in at least several normal tissues, and testis-specific or predominant expression means significant PCR products observed only in testis or in testis and at most two additional normal tissues (also see Table 6).

FIG. 6 shows mRNA expression of CT46 in tumor cell lines and specimens. The expression level was determined by real-time RT-PCR and expressed as a sample percentage of the testicular expression level. Each open circle represents one sample. Cell lines were for melanoma, small cell lung cancer (SCLC), neuroblastoma, and colon cancer, whereas RNA from primary tumor specimens was from non-small cell lung cancer (NSCLC), breast cancer, bladder cancer, esophageal cancer, endometrial cancer, and colon cancer.

FIG. 7 shows amino acid sequence alignments. FIG. 7A shows an alignment between CT46 (amino acids 15-394 of SEQ ID NO:25) and prototype HORMA-domain containing protein KOG4652 (SEQ ID NO:35). FIG. 7B shows an alignment between CT46 (amino acids 1-241 of SEQ ID NO:25) and the homologous MGC26710 hypothetical protein (amino acids 1-249 of SEQ ID NO:34). Identical sequences are indicated, conservative amino acid changes are shown as +, and gaps are indicated with dashes.

FIG. 8 shows reactivity of antibodies in sera of non-small cell lung cancer patients with CT45 protein.

FIG. 9 shows reactivity of antibodies in sera of non-small cell lung cancer patients with CT46-HORMAD1 protein.

### BRIEF DESCRIPTION OF TABLES

Table 1—chromosome distribution of CT and CT-like genes.
Table 2—expression of CT and CT-like genes in normal tissue.
Table 3—mRNA expression of CT and CT-like genes in different cell lines.
Table 4—primer and probe sequences used for quantitative RT-PCR of CT genes. The sequences are: THEG forward primer, reverse primer and probe, SEQ ID NOs:10-12, respectively; NALP4 forward primer, reverse primer and probe, SEQ ID NOs:13-15, respectively; COXVIB2 forward primer, reverse primer and probe, SEQ ID NOs:16-18, respectively; LOC348120 forward primer, reverse primer and probe, SEQ ID NOs:19-21; and CT45 forward primer, reverse primer and probe, SEQ ID NOs:22-24.
Table 5—CT candidate genes selected for RT-PCR validation.
Table 6—expression of CT candidate genes in normal tissues.
Table 7—expression of CT candidate genes in a “CT-rich” cell line panel.

### DESCRIPTION OF THE SEQUENCES

SEQ ID NO:1 Full-length cDNA sequence of CT45, transcript variant 1 (NM_{—}152582.3): The coding sequence begins at nucleotide residue 246 and ends at nucleotide residue 815.
SEQ ID NO:2 Full-length cDNA sequence of CT45, transcript variant 2 (AK098689.1). The coding sequence begins at nucleotide residue 91 and ends at nucleotide residue 660.
SEQ ID NO:3 Predicted protein sequence of CT45 (Hypothetical protein MGC27005).
SEQ ID NO:4 Amino acid sequence of CT45 protein DEAD box helicase domain, residues 65-185.
SEQ ID NO:5 Amino acid sequence of a portion of the LOC203522 protein
SEQ ID NO:6 Amino acid sequence of a portion of the CT45 protein, from residue 126.
SEQ ID NO:7 Amino acid sequence of CT45-like protein LOC203522 (NM_{—}182540).
SEQ ID NO:8 Amino acid sequence for DDX26 (NM_{—}012141).
SEQ ID NO:9 Amino acid sequence of SAGE protein (NP_{—}061136.1).
SEQ ID NO:10 Nucleotide sequence for forward primer THEE.
SEQ ID NO:11 Nucleotide sequence for reverse primer THEG.
SEQ ID NO:12 Nucleotide sequence for probe THEG.
SEQ ID NO:13 Nucleotide sequence for forward primer NALP4.
SEQ ID NO:14 Nucleotide sequence for reverse primer NALP4.
SEQ ID NO:15 Nucleotide sequence for probe NALP4.
SEQ ID NO:16 Nucleotide sequence for forward primer COXVIB2.
SEQ ID NO:17 Nucleotide sequence for reverse primer COXVIB2.
SEQ ID NO:18 Nucleotide sequence for probe COXVIB2.
SEQ ID NO:19 Nucleotide sequence for forward primer LOC348120 (Hs.116287).
SEQ ID NO:20 Nucleotide sequence for reverse primer LOC348120 (Hs.116287).
SEQ ID NO:21 Nucleotide sequence for probe LOC348120 (Hs.116287).
SEQ ID NO:22 Nucleotide sequence for forward primer CT45.
SEQ ID NO:23 Nucleotide sequence for reverse primer CT45.
SEQ ID NO:24 Nucleotide sequence for probe CT45.
SEQ ID NO:25 Amino acid sequence of the CT46 protein (NM_{—}032132).
SEQ ID NO:26 Nucleotide sequence encoding the CT46 protein (NM_{—}173493.1)
SEQ ID NO:27 Amino acid sequence for HORMA domain (KOG4652).
SEQ ID NO:28 Amino acid sequence of a portion of the DDX26 protein, from residue 810.
SEQ ID NO:29 Amino acid sequence of a portion of the SAGE protein, from residue 880.
SEQ ID NO:30 Nucleotide sequence for CT46 transcript variant 2.
SEQ ID NO:31 Amino acid sequence of the 60 amino acid polypeptide encoded by CT46 transcript variant 2 from the same initiation codon as used in transcript variant 1 (SEQ ID NO:26).
SEQ ID NO:32 Amino acid sequence of the 323 amino acid polypeptide encoded by CT46 transcript variant 2 from an alternative initiation codon.
SEQ ID NO:33 Nucleotide sequence encoding the MGC26710 protein (NM_{—}152510).
SEQ ID NO:34 Amino acid sequence of the MGC26710 protein (NM_{—}152510).
SEQ ID NO:35 Amino acid sequence of KOG4652, HORMA domain.

### DETAILED DESCRIPTION OF THE INVENTION

In the first part of this study, we identified genes with massively parallel signature sequencing (MPSS) tags only in testis but not in other normal somatic tissues, and the mRNA expression patterns of these genes in normal tissue and in cancer cell lines were then investigated by RT-PCR. By this approach, we have identified more than a dozen cancer-testis (CT) and CT-like genes, providing targets for cancer vaccines. We also searched for CT and CT-like genes by analyzing EST database records for genes with testis-predominant expression, followed by investigation of their expression in tumors by RT-PCR analysis. Another CT gene was identified using this methodology.

MPSS data on 32 normal tissues were analyzed, and a list of testis-specific genes was compiled, consisting of genes that showed 10 or more MPSS tags in the two testicular samples combined. This list contains 1056 genes in total, with 39 genes located on chromosome X. Among the genes on chromosome X were several known CT gene families, including NY-ESO-1, LAGE1, MAGE-B1, -B2, and -B4, GAGE1, GAGE2, and PAGE5, validating the potential of this technique in finding new CT antigen genes.

The unknown genes on chromosome X were further evaluated, first by searching corresponding EST sequences in the public database. Genes were considered CT-candidate genes if they have ESTs derived from a) testis, ovary, and/or placenta, b) any tumor (except germ cell tumor), and c) no more than two somatic tissues. The exon-intron structures of the CT-candidate genes were then defined by BLASTN. Trans-intronic PCR primer pairs were made for each gene, and the mRNA expression of these genes in normal and tumor cells were then evaluated experimentally by RT-PCR analysis. Two RNA panels were tested sequentially, the first one consisting of 16 normal tissues and the second one of 21 cancer cell lines. The normal tissues tested were brain, colon, heart, kidney, leukocytes, liver, lung, ovary, pancreas, placenta, prostate, skeletal muscle, small intestine, spleen, thymus, and testis. The cancer cell lines tested included 7 melanoma (SK-MEL-10, -24, -37, -49, -55, -80, -128), 4 small cell lung cancer (NCI-H82, -H128, -H187, -H740), 3 non-small cell lung cancer (SK-LC-5, -14, -17), 3 colon cancer (SW403, HCT15, LS174T), 1 renal cancer (SK-RCC-1), 1 hepatocellular carcinoma (SK-HEP-1), 1 bladder cancer (T24), and 1 sarcoma (SW982). These cell lines were selected as a “CT-rich” panel, with each of them positive for one or more well-characterized CT genes.

Using these screening methodologies, two genes in the X chromosome emerged as new CT genes, designated CT45 and CT46, following the proposed CT nomenclature system. CT46/HORMAD1 (Hs.160594, NM_{—}173493.1, SEQ ID NOs:25 and 26) is a single copy gene on Xq28, whereas CT45, encoding hypothetical protein MGC27005 (Hs. 460933, NM_{—}152582.3, SEQ ID NOs:1-3), is located on chromosome Xq26.3 and was found to be a multigene family. An alternative transcript of CT46/HORMAD1 (transcript variant 2) was identified (SEQ ID NO:30), as were two translation products of the alternative transcript (SEQ ID NOs:31 and 32). The foregoing are referred to herein as cancer-testis antigens.

The invention relates, in part, to the cancer-testis antigens defined herein and the nucleic acid molecules that encode them. The invention further relates to the use of the nucleic acid molecules, polypeptides and fragments thereof in methods and compositions for the diagnosis and treatment of diseases, such as cancer.

The invention involves diagnosing or monitoring cancer in a subject by determining the presence or amount of an immune response to one or more cancer-testis antigens of the invention. In preferred embodiments, this determination is performed by assaying a biological sample obtained from the subject, preferably serum, blood, or lymph node fluid, for the presence of antibodies against the cancer-testis antigens described herein. This determination may also be performed by assaying a tissue or cells from the subject for the presence of one or more cancer-testis antigens (or nucleic acid molecules that encode these antigens) described herein. In another embodiment, the presence of antibodies against at least one additional cancer antigen is determined for diagnosis of cancer. The additional antigen may be a cancer-testis antigen as described herein or may be some other cancer-associated antigen. Thus tissues or cells from the subject can be assayed for the presence of a plurality of cancer-testis antigens.

Measurement of the immune response against one of the cancer-testis antigens over time by sequential determinations permits monitoring of the disease and/or the effects of a course of treatment. For example, a sample, such as serum, blood, or lymph node fluid, may be obtained from a subject, tested for an immune response to one of the cancer-testis antigens, and at a second, subsequent time, another sample, may be obtained from the subject and similarly tested. The results of the first and second (or subsequent) tests can be compared as a measure of the onset, regression or progression of cancer, or, if cancer treatment was undertaken during the interval between obtaining the samples, the effectiveness of the treatment may be evaluated by comparing the results of the two tests. In preferred embodiments the cancer-testis antigens are bound to a substrate. In other preferred embodiments the immune response of the biological sample to the cancer-testis antigens is determined with ELISA. Other methods will be apparent to one of skill in the art.

Diagnostic methods of the invention also involve determining the aberrant expression of one or more of the cancer-testis antigens described herein or the nucleic acid molecules that encode them. Such determinations can be carried out via any standard nucleic acid assay, including the polymerase chain reaction or assaying with hybridization probes, which may be labeled, or by assaying biological samples with binding partners (e.g., antibodies) for cancer-testis antigens using standard methodologies.

The diagnostic methods of the invention can be used to detect the presence of a disorder associated with aberrant expression of a cancer-testis molecule, as well as to assess the progression and/or regression of the disorder such as in response to treatment (e.g., chemotherapy, radiation). According to this aspect of the invention, the method for diagnosing a disorder characterized by aberrant expression of a cancer-testis molecule involves: detecting expression of a cancer-testis molecule in a first biological sample obtained from a subject, wherein differential expression of the cancer-testis molecule compared to a control sample indicates that the subject has a disorder characterized by aberrant expression of a cancer-testis molecule, such as cancer.

As used herein, “aberrant expression” of a cancer-testis antigen is intended to include any expression that is different by a statistically significant amount from the expected amount of expression. For example, expression of a cancer-testis molecule (i.e., the cancer-testis antigen or the nucleic acid molecules that encode it) in a tissue that is not expected to express the cancer-testis molecule would be included in the definition of “aberrant expression”. Likewise, expression of the cancer-testis molecule that is determined to be expressed at a significantly higher or lower level than expected is also included. Therefore, a determination of the level of expression (i.e., the presence of amount) of one or more of the cancer-testis antigens and/or the nucleic acids that encode them is diagnostic of cancer if the level of expression is above a baseline level determined for that tissue type. The baseline level of expression can be determined using standard methods known to those of skill in the art. Such methods include, for example, assaying a number of histologically normal tissue samples from subjects that are clinically normal (i.e., do not have clinical signs of cancer in that tissue type) and determining the mean level of expression for the samples.

The level of expression of the nucleic acid molecules of the invention or the antigens they encode can indicate cancer in the tissue when the level of expression is significantly more in the tissue than in a control sample. In some embodiments, a level of expression in the tissues that is at least about 5%, 10%, 20%, 30%, 40%, 50%, 60%, 70%, 80%, 90%, 100%, 150%, 200%, 250%, 300%, 400%, or 500% more than the level of expression in the control tissue indicates cancer in the tissue. Alternatively, expression of the CT antigens or nucleic acids in a non-testis tissue that is at least about 0.1%, 0.2%, 0.3%, 0.4%, 0.5%, 0.6%, 0.7%, 0.8%, 0.9%, more preferably at least about 1.0%, 2.0%, 3.0%, 4.0%, 5.0%, 6.0%, 7.0%, 8.0% or 9.0%, or most preferably at least about 10.0%, 11%, 12%, 13%, 14%, 15%, 16%, 17%, 18%, 19%, or 20% of the level of expression in testis, indicates cancer in the tissue.

As used herein the term “control” means predetermined values, and also means samples of materials tested in parallel with the experimental materials. Examples include samples from control populations or control samples generated through manufacture to be tested in parallel with the experimental samples.

As used herein the term “control” includes positive and negative controls which may be a predetermined value that can take a variety of forms. The control(s) can be a single cut-off value, such as a median or mean, or can be established based upon comparative groups, such as in groups having normal amounts of cancer-testis molecules of the invention and groups having abnormal amounts of cancer-testis molecules of the invention. Another example of a comparative group is a group having a particular disease, condition and/or symptoms and a group without the disease, condition and/or symptoms. Another comparative group is a group with a family history of a particular disease and a group without such a family history of the particular disease. The predetermined control value can be arranged, for example, where a tested population is divided equally (or unequally) into groups, such as a low-risk group, a medium-risk group and a high-risk group or into quadrants or quintiles, the lowest quadrant or quintile being individuals with the lowest risk or lowest expression levels of a cancer-testis molecule of the invention that is up-regulated in cancer and the highest quadrant or quintile being individuals with the highest risk or highest expression levels of a cancer-testis molecule of the invention that is up-regulated in cancer.

The predetermined value of a control will depend upon the particular population selected. For example, an apparently healthy population will have a different “normal” cancer-testis molecule expression level range than will a population which is known to have a condition characterized by aberrant expression of the cancer-testis molecule. Accordingly, the predetermined value selected may take into account the category in which an individual falls. Appropriate ranges and categories can be selected with no more than routine experimentation by those of ordinary skill in the art. Typically the control will be based on apparently healthy individuals in an appropriate age bracket. As used herein, the term “increased expression” means a higher level of expression relative to a selected control.

The invention involves in some aspects diagnosing or monitoring cancer by determining the level (i.e., presence or amount) of expression of one or more cancer-testis nucleic acid molecules and/or determining the level (i.e., presence or amount) of expression of one or more cancer-testis polypeptides they encode. In some important embodiments, this determination is performed by assaying a tissue sample from a subject for the level of expression of one or more cancer-testis nucleic acid molecules or for the level of expression of one or more cancer-testis polypeptides encoded by the nucleic acid molecules of the invention.

The expression of the molecules of the invention may be determined using routine methods known to those of ordinary skill in the art. These methods include, but are not limited to: direct RNA amplification, reverse transcription of RNA to cDNA, real-time RT-PCR, amplification of cDNA, hybridization, and immunologically based assay methods, which include, but are not limited to immunohistochemistry, antibody sandwich capture assay, ELISA, and enzyme-linked immunospot assay (EliSpot assay). For example, the determination of the presence of level of nucleic acid molecules of the invention in a subject or tissue can be carried out via any standard nucleic acid determination assay, including the polymerase chain reaction, or assaying with labeled hybridization probes. Such hybridization methods include, but are not limited to, microarray techniques.

These methods of determining the presence and/or level of the molecules of the invention in cells and tissues may include use of labels to monitor the presence of the molecules of the invention. Such labels may include, but are not limited to radiolabels or chemiluminescent labels, which may be utilized to determine whether a molecule of the invention is expressed in a cell or tissue, and to determine the level of expression in the cell or tissue. For example, a fluorescently labeled or radiolabeled antibody that selectively binds to a polypeptide of the invention may be contacted with a tissue or cell to visualize the polypeptide in vitro or in vivo. These and other in vitro and in vivo imaging methods for determining the presence of the nucleic acid and polypeptide molecules of the invention are well known to those of ordinary skill in the art.

The invention includes kits for assaying the presence of cancer-testis antigens and/or antibodies that specifically bind to cancer-testis polypeptides. An example of such a kit may include the above-mentioned polypeptides bound to a substrate, for example a dipstick, which is dipped into a blood or body fluid sample of a subject. The surface of the substrate may then be processed using procedures well known to those of skill in the art, to assess whether specific binding occurred between the polypeptides and agents (e.g., antibodies) in the subject's sample. For example, procedures may include, but are not limited to, contact with a secondary antibody, or other method that indicates the presence of specific binding.

Another example of a kit may include an antibody or antigen-binding fragment thereof, that binds specifically to a cancer-testis antigen. The antibody or antigen-binding fragment thereof, may be applied to a tissue or cell sample from a patient with cancer and the sample then processed to assess whether specific binding occurs between the antibody and an antigen or other component of the sample. In addition, the antibody or antigen-binding fragment thereof, may be applied to a body fluid sample, such as serum, from a subject, either suspected of having cancer, diagnosed with cancer, or believed to be free of cancer. As will be understood by one of skill in the art, such binding assays may also be performed with a sample or object contacted with an antibody and/or cancer-testis antigen that is in solution, for example in a 96-well plate or applied directly to an object surface.

Another example of a kit of the invention is a kit that provides components necessary to determine the level of expression of one or more cancer-testis nucleic acid molecules of the invention. Such components may include primers useful for amplification of one or more cancer-testis nucleic acid molecules and/or other chemicals for PCR amplification.

Another example of a kit of the invention is a kit that provides components necessary to determine the level of expression of one or more cancer-testis nucleic acid molecules of the invention using a method of hybridization.

The foregoing kits can include instructions or other printed material on how to use the various components of the kits for diagnostic purposes.

As used herein, the “nucleic acid molecules that encode” means the nucleic acid molecules that code for the cancer-testis polypeptides or immunogenic fragments thereof. These nucleic acid molecules may be DNA or may be RNA (e.g. mRNA). The cancer-testis nucleic acid molecules of the invention also encompass variants of the nucleic acid molecules described herein. These variants may be splice variants or allelic variants of certain sequences provided. Variants of the nucleic acid molecules of the invention are intended to include homologs and alleles which are described further below. Further, as used herein, the term “cancer-testis molecules” includes cancer-testis antigens (polypeptides and fragments thereof) as well as cancer-testis nucleic acids. In all embodiments, human cancer-testis antigens and the encoding nucleic acid molecules thereof, are preferred.

In one aspect, the invention provides isolated nucleic acid molecules that encode the cancer-testis antigens defined herein. The isolated nucleic acid molecules of this aspect of the invention comprise: (a) nucleotide sequences selected from the group consisting of nucleotide sequences set forth as SEQ ID NO:1, SEQ ID NO:2 and SEQ ID NO:26 (b) isolated nucleic acid molecules which hybridize under highly stringent conditions to the nucleic acid molecules of (a) and which code for a cancer-testis antigen, (c) nucleic acid molecules that differ from (a) or (b) due to the degeneracy of the genetic code, and (d) complements of (a), (b) or (c). In certain preferred embodiments, the nucleic acid molecules are those that encode a polypeptide having an amino acid sequence as set forth in SEQ ID NO:3, or a fragment thereof, or nucleic acid molecules comprising a nucleotides sequence that is at least about 90%, more preferably at least about 93%, more preferably at least about 95%, more preferably at least about 97%, still more preferably at least about 99% identical to a nucleotide sequence that encodes SEQ ID NO:3.

As used herein the term “isolated nucleic acid molecule” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

The cancer-testis nucleic acid molecules of the invention are also intended to encompass homologs and alleles which can be identified by conventional techniques. Identification of human homologs and homologs of other organisms (i.e., orthologs) of cancer-testis polypeptides will be familiar to those of skill in the art. In general, nucleic acid hybridization is a suitable method for identification of homologous sequences of another species (e.g., human, cow, sheep), which correspond to a known sequence. Standard nucleic acid hybridization procedures can be used to identify related nucleic acid sequences of selected percent identity. For example, one can construct a library of cDNAs reverse transcribed from the mRNA of a selected tissue and use the nucleic acids that encode cancer-testis antigens identified herein to screen the library for related nucleotide sequences. The screening preferably is performed using high-stringency conditions to identify those sequences that are closely related by sequence identity. Nucleic acids so identified can be translated into polypeptides and the polypeptides can be tested for activity.

The term “high stringency” as used herein refers to parameters with which the art is familiar. Nucleic acid hybridization parameters may be found in references that compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, high-stringency conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄ (pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C. The temperature of the wash may be adjusted to provide different levels of stringency. For example the wash can be performed at temperatures of 42° C., 42° C., 42° C., 42° C., 42° C., 42° C., or 68° C. The skilled artisan would be able to adjust the temperature to determine the optimum temperature as required.

There are other conditions, reagents, and so forth that can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of the cancer-testis nucleic acids of the invention (e.g., by using lower stringency conditions). The skilled artisan also is familiar with the methodology for screening cells and libraries for expression of such molecules, which then are routinely isolated, followed by isolation of the pertinent nucleic acid molecule and sequencing.

Optimal alignment of sequences for comparison may alternatively be conducted using programs such as BLAST, publicly available on the National Library of Medicine website. Other programs such as UniGene (The National Library of Medicine website), SAGE Anatomic Reviewer and its Virtual Northern tool, (The Cancer Genome Anatomy Project CGAP website) are also publicly available. Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

In general, homologs and alleles typically will share at least 90% nucleotide identity and/or at least 95% amino acid identity to the sequences of cancer-testis nucleic acids and polypeptides, respectively, in some instances will share at least 95% nucleotide identity and/or at least 97% amino acid identity, in other instances will share at least 97% nucleotide identity and/or at least 98% amino acid identity, in other instances will share at least 99% nucleotide identity and/or at least 99% amino acid identity, and in other instances will share at least 99.5% nucleotide identity and/or at least 99.5% amino acid identity. The homology can be calculated using various, publicly available software tools developed by NCBI (Bethesda, Md.) that can be obtained through the internet. Exemplary tools include the BLAST system available from the website of the National Center for Biotechnology Information (NCBI) at the National Institutes of Health. Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

In another aspect of the invention, unique fragments are provided which include unique fragments of the nucleotide sequences of the invention and complements thereof. The invention, in a preferred embodiment, provides unique fragments of SEQ ID NO:1, SEQ ID NO.2, SEQ ID NO:26 or SEQ ID NO:30 and complements thereof. A unique fragment is one that is a ‘signature’ for the larger nucleic acid. It, for example, is long enough to assure that its precise sequence is not found in molecules outside of the nucleic acid molecules that encode the cancer-testis antigens defined above. Those of ordinary skill in the art may apply no more than routine procedures to determine if a fragment is unique within the human genome. For polypeptides of the invention (e.g., SEQ ID NOs:3, 25, 31, 32), the fragment can be at least about 5, 6, 7, 8, 9, 10, 11, 12, 13, 14, 15, 16, 17, 18, 19, 20, 25, 30, 35, 40, 45, 50, 75, or 100 amino acids in length.

Unique fragments can be used as probes in Southern blot assays to identify such nucleic acid molecules, or can be used as probes in amplification assays such as those employing the polymerase chain reaction (PCR), including, but not limited to RT-PCR and RT-real-time PCR. As known to those skilled in the art, large probes such as 200 nucleotides or more are preferred for certain uses such as Southern blots, while smaller fragments will be preferred for uses such as PCR. Unique fragments also can be used to produce fusion proteins for generating antibodies or determining binding of the polypeptide fragments, or for generating immunoassay components. Likewise, unique fragments can be employed to produce nonfused fragments of the cancer-testis polypeptides useful, for example, in the preparation of antibodies and in immunoassays.

In screening for cancer-testis antigen genes, a Southern blot may be performed using the foregoing conditions, together with a detectably labeled probe (e.g., radioactive or chemiluminescent probes). After washing the membrane to which the DNA is finally transferred, the signal from the detectably labeled probe can be detected, for example by placing the membrane against X-ray film or analyzing it using a phosphorimager device to detect the detectable signal. In screening for the expression of cancer-testis antigen nucleic acids, Northern blot hybridizations using the foregoing conditions can be performed on samples taken from cancer patients or subjects suspected of having a condition characterized by abnormal cell proliferation or neoplasia. Amplification protocols such as polymerase chain reaction using primers that hybridize to the sequences presented also can be used for detection of the cancer-testis antigen genes or expression thereof.

Identification of related sequences can also be achieved using polymerase chain reaction (PCR) and other amplification techniques suitable for cloning related nucleic acid sequences. Preferably, PCR primers are selected to amplify portions of a nucleic acid sequence believed to be conserved (e.g., a catalytic domain, a DNA-binding domain, etc.). Again, nucleic acids are preferably amplified from a tissue-specific library (e.g., testis).

The invention also includes degenerate nucleic acids that include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating cancer-testis polypeptide. Similarly, nucleotide sequence triplets which encode other amino acid residues include, but are not limited to: CCA, CCC, CCG, and CCT (proline codons); CGA, CGC, CGG, CGT, AGA, and AGG (arginine codons); ACA, ACC, ACG, and ACT (threonine codons); AAC and AAT (asparagine codons); and ATA, ATC, and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules, which include additions, substitutions and deletions of one or more nucleotides (preferably 1-20 nucleotides). In preferred embodiments, these modified nucleic acid molecules and/or the polypeptides they encode retain at least one activity or function of the unmodified nucleic acid molecule and/or the polypeptides, such as antigenicity, receptor binding, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under stringent conditions known to one of skill in the art.

For example, modified nucleic acid molecules that encode polypeptides having single amino acid changes can be prepared. Each of these nucleic acid molecules can have one, two or three nucleotide substitutions exclusive of nucleotide changes corresponding to the degeneracy of the genetic code as described herein. Likewise, modified nucleic acid molecules that encode polypeptides having two amino acid changes can be prepared which have, e.g., 2-6 nucleotide changes. Numerous modified nucleic acid molecules like these will be readily envisioned by one f skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of activity or structural relation to the nucleic acids and/or polypeptides disclosed herein. As used herein the terms: “deletion”, “addition”, and “substitution” mean deletion, addition, and substitution changes to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more nucleic acids of a sequence of the invention.

According to yet another aspect of the invention, an expression vector comprising any of the isolated nucleic acid molecules of the invention, preferably operably linked to a promoter, is provided. In a related aspect, host cells transformed or transfected with such expression vectors also are provided. As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids, and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art, e.g., -galactosidase or alkaline phosphatase, and genes which visibly affect the phenotype of transformed or transfected cells, hosts, colonies or plaques, e.g., green fluorescent protein. Preferred vectors are those capable of autonomous replication and expression of the structural gene products present in the DNA segments to which they are operably joined.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation; (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region is capable of effecting transcription of that DNA sequence such that the resulting transcript can be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Often, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

It will also be recognized that the invention embraces the use of the cancer-testis nucleic acid molecules and genomic sequences in expression vectors, as well as to transfect host cells and cell lines, be these prokaryotic, e.g., E. coli, or eukaryotic, e.g., CHO cells, COS cells, yeast expression systems, and recombinant baculovirus expression in insect cells. Especially useful are mammalian cells such as human, mouse, hamster, pig, goat, primate, etc. They may be of a wide variety of tissue types, including mast cells, fibroblasts, oocytes, and lymphocytes, and may be primary cells and cell lines. Specific examples include dendritic cells, peripheral blood leukocytes, bone marrow stem cells and embryonic stem cells. The expression vectors require that the pertinent sequence, i.e., those nucleic acids described supra, be operably linked to a promoter.

The invention, in one aspect, also permits the construction of cancer-testis antigen gene “knock-outs” and “knock-ins” in cells and in animals, providing materials for studying certain aspects of cancer and immune system responses to cancer.

Expression vectors containing all the necessary elements for expression are commercially available and known to those skilled in the art. See, e.g., Sambrook et al., Molecular Cloning: A Laboratory Manual, Second Edition, Cold Spring-Harbor Laboratory Press, 1989. Cells are genetically engineered by the introduction into the cells of heterologous DNA or RNA encoding a cancer-testis antigen, a mutant cancer-testis antigen, fragments, or variants thereof. The heterologous DNA or RNA is placed under operable control of transcriptional elements to permit the expression of the heterologous DNA in the host cell.

Preferred systems for mRNA expression in mammalian cells are those such as pcDNA/V5-GW/D-TOPO® and pcDNA3.1 (Invitrogen) that contain a selectable marker (which facilitates the selection of stably transfected cell lines) and contain the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element. Another expression vector is the pEF-BOS plasmid containing the promoter of polypeptide Elongation Factor 1, which stimulates efficiently transcription in vitro. The plasmid is described by Mizushima and Nagata (Nuc. Acids Res. 18:5322, 1990), and its use in transfection experiments is disclosed by, for example, Demoulin (Mol. Cell. Biol. 16:4710-4716, 1996). Still another preferred expression vector is an adenovirus, described by Stratford-Perricaudet, which is defective for E1 and E3 proteins (J. Clin. Invest. 90:626-630, 1992). The use of the adenovirus as an Adeno.P1A recombinant is described by Warnier et al., in intradermal injection in mice for immunization against P1A (Int. J. Cancer, 67:303-310, 1996).

The invention also embraces kits termed expression kits, which allow the artisan to prepare a desired expression vector or vectors. Such expression kits include at least separate portions of each of the previously discussed coding sequences. Other components may be added, as desired, as long as the previously mentioned sequences, which are required, are included.

The invention also includes kits for amplification of a cancer-testis antigen nucleic acid, including at least one pair of amplification primers which hybridize to a cancer-testis nucleic acid. The primers preferably are about 12, 13, 14, 15, 16, 17, 18, 19, 20, 21, 22, 23, 24, 25, 26, 27, 28, 29, 30, 31 or 32 nucleotides in length and are non-overlapping to prevent formation of “primer-dimers”. One of the primers will hybridize to one strand of the cancer-testis nucleic acid and the second primer will hybridize to the complementary strand of the cancer-testis nucleic acid, in an arrangement which permits amplification of the cancer-testis nucleic acid. Selection of appropriate primer pairs is standard in the art. For example, the selection can be made with assistance of a computer program designed for such a purpose, optionally followed by testing the primers for amplification specificity and efficiency.

The invention, in another aspect provides isolated polypeptides (including whole proteins and partial proteins) encoded by the foregoing cancer-testis nucleic acids. Examples of the amino acid sequences encoded by the foregoing cancer-testis nucleic acids are set forth as SEQ ID NOs: 3, 4, 6, 25, 31 and 32. The amino acids of the invention are also intended to encompass amino acid sequences that result from the translation of the nucleic acid sequences provided herein in a different reading frame. In preferred embodiments of the invention a polypeptide is provided which comprises the amino acid sequence set forth as SEQ ID NO: 3, 4, 6 or 25. In a particularly preferred embodiment, a polypeptide is provided which comprises the amino acid sequence set forth as SEQ ID NO: 3 or a fragment thereof. In another particularly preferred embodiment, the fragment comprises 8 or more amino acids. In a further particularly preferred embodiment, polypeptides which comprise the amino acid sequences set forth as SEQ ID NO:4, SEQ ID NO:6 or SEQ ID NO:25 are provided. Such polypeptides are useful, for example, alone or as fusion proteins to generate antibodies, and as components of an immunoassay or diagnostic assay. Immunogenic cancer-testis polypeptides can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein.

Fragments of the immunogenic cancer-testis polypeptides (including immunogenic peptides) also can be synthesized chemically using well-established methods of peptide synthesis. Thus, fragments of the disclosed polypeptides are useful for eliciting an immune response and for assaying for the presence of antibodies, or other similar molecules such as T cell receptors. In one embodiment fragments of a polypeptide which comprises SEQ ID NO:3 that are at least eight amino acids in length and exhibit immunogenicity are provided. The fragments may be any length from 8 amino acids up to one amino acid less than the full length size of polypeptide Specific embodiments provide fragments of a polypeptide which comprise the polypeptide sequences set forth as SEQ ID NO: 4 or 6. In other embodiments of a like nature, fragments of CT46 polypeptides (SEQ ID NOs: 25, 31, 32) that are at least eight amino acids in length and exhibit immunogenicity are provided.

Fragments of a polypeptide preferably are those fragments that retain a distinct functional capability of the polypeptide. Functional capabilities that can be retained in a fragment of a polypeptide include interaction with antibodies or MHC molecules (e.g. immunogenic fragments), interaction with other polypeptides or fragments thereof, selective binding of nucleic acids or proteins, and enzymatic activity. One important activity is the ability to provoke in a subject an immune response. As will be recognized by those skilled in the art, the size of the fragment that can be used for inducing an immune response will depend upon factors such as whether the epitope recognized by an antibody is a linear epitope or a conformational epitope or the particular MHC molecule that binds to and presents the fragment (e.g. HLA class I or II). Thus, some immunogenic fragments of cancer-testis polypeptides will consist of longer segments while others will consist of shorter segments, (e.g., about 8, 9, 10, 11, 12, 13, 14, 15, 16 or more amino acids long, including each integer up to the full length of the cancer-testis polypeptides). Those skilled in the art are well versed in methods for selecting immunogenic fragments of polypeptides.

The invention embraces variants of the cancer-testis polypeptides described above. As used herein, a “variant” of a cancer-testis antigen polypeptide is a polypeptide which contains one or more modifications to the primary amino acid sequence of a cancer-testis polypeptide. Modifications which create a cancer-testis antigen variant can be made to a cancer-testis polypeptide 1) to reduce or eliminate an activity of a cancer-testis polypeptide; 2) to enhance a property of a cancer-testis polypeptide, such as protein stability in an expression system or the stability of protein-protein binding; 3) to provide a novel activity or property to a cancer-testis polypeptide, such as addition of an antigenic epitope or addition of a detectable moiety; or 4) to provide equivalent or better binding to a MHC molecule.

Modifications to a cancer-testis polypeptide are typically made to the nucleic acid which encodes the cancer-testis polypeptide, and can include deletions, point mutations, truncations, amino acid substitutions and additions of amino acids or non-amino acid moieties. Alternatively, modifications can be made directly to the polypeptide, such as by cleavage, addition of a linker molecule, addition of a detectable moiety, such as biotin, addition of a fatty acid, and the like. Modifications also embrace fusion proteins comprising all or part of the cancer-testis antigen amino acid sequence. One of skill in the art will be familiar with methods for predicting the effect on protein conformation of a change in protein sequence, and can thus “design” a variant cancer-testis polypeptide according to known methods. One example of such a method is described by Dahiyat and Mayo in Science 278:82-87, 1997, whereby proteins can be designed de novo. The method can be applied to a known protein to vary only a portion of the polypeptide sequence. By applying the computational methods of Dahiyat and Mayo, specific variants of a cancer-testis polypeptide can be proposed and tested to determine whether the variant retains a desired conformation.

In general, variants include cancer-testis polypeptides which are modified specifically to alter a feature of the polypeptide unrelated to its desired physiological activity. For example, cysteine residues can be substituted or deleted to prevent unwanted disulfide linkages. Similarly, certain amino acids can be changed to enhance expression of a cancer-testis polypeptide by eliminating proteolysis by proteases in an expression system (e.g., dibasic amino acid residues in yeast expression systems in which KEX2 protease activity is present).

Mutations of a nucleic acid which encode a cancer-testis polypeptide preferably preserve the amino acid reading frame of the coding sequence, and preferably do not create regions in the nucleic acid which are likely to hybridize to form secondary structures, such a hairpins or loops, which can be deleterious to expression of the variant polypeptide.

Mutations can be made by selecting an amino acid substitution, or by random mutagenesis of a selected site in a nucleic acid which encodes the polypeptide. Variant polypeptides are then expressed and tested for one or more activities to determine which mutation provides a variant polypeptide with the desired properties. Further mutations can be made to variants (or to non-variant cancer-testis polypeptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a cancer-testis antigen gene or cDNA clone to enhance expression of the polypeptide. The activity of variants of cancer-testis polypeptides can be tested by cloning the gene encoding the variant cancer-testis polypeptide into a bacterial or mammalian expression vector, introducing the vector into an appropriate host cell, expressing the variant cancer-testis polypeptide, and testing for a functional capability of the cancer-testis polypeptides as disclosed herein. For example, the variant cancer-testis polypeptide can be tested for reaction with autologous or allogeneic sera. Preparation of other variant polypeptides may favor testing of other activities, as will be known to one of ordinary skill in the art.

The skilled artisan will also realize that conservative amino acid substitutions may be made in immunogenic cancer-testis polypeptides to provide functionally equivalent variants, or homologs of the foregoing polypeptides, i.e., the variants retain the functional capabilities of the immunogenic cancer-testis polypeptides. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants or homologs of the cancer-testis polypeptides include conservative amino acid substitutions of in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the cancer-testis antigens disclosed herein and retain the specific antibody-binding characteristics of the antigens.

Likewise, upon determining that a peptide derived from a cancer-testis polypeptide is presented by an MHC molecule and recognized by antibodies or T lymphocytes (e.g., helper T cells or CTLs), one can make conservative amino acid substitutions to the amino acid sequence of the peptide, particularly at residues which are thought not to be direct contact points with the MHC molecule. For example, methods for identifying functional variants of HLA class II binding peptides are provided in a published PCT application of Strominger and Wucherpfennig (PCT/US96/03182). Peptides bearing one or more amino acid substitutions also can be tested for concordance with known HLA/MHC motifs prior to synthesis using, e.g. the computer program described by D'Amaro and Drijfhout (D'Amaro et al., Human Immunol. 43:13-18, 1995; Drijfhout et al., Human Immunol. 43:1-12, 1995). The substituted peptides can then be tested for binding to the MHC molecule and recognition by antibodies or T lymphocytes when bound to MHC. These variants can be tested for improved stability and are useful, inter alia, in vaccine compositions.

Conservative amino-acid substitutions in the amino acid sequence of cancer-testis polypeptides to produce functionally equivalent variants of cancer-testis polypeptides typically are made by alteration of a nucleic acid encoding a cancer-testis polypeptide. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a cancer-testis polypeptide. Where amino acid substitutions are made to a small unique fragment of a cancer-testis polypeptide, such as an antigenic epitope recognized by autologous or allogeneic sera or T lymphocytes, the substitutions can be made by directly synthesizing the peptide. The activity of functionally equivalent variants of cancer-testis polypeptides can be tested by cloning the gene encoding the altered cancer-testis polypeptide into a bacterial or mammalian expression vector, introducing the vector into an appropriate host cell, expressing the altered polypeptide, and testing for a functional capability of the cancer-testis polypeptides as disclosed herein. Peptides that are chemically synthesized can be tested directly for function, e.g., for binding to antisera recognizing associated antigens.

As used herein, a “subject” is preferably a human, non-human primate, cow, horse, pig, sheep, goat, dog, cat or rodent. In all embodiments, human subjects are preferred. In some embodiments, the subject is suspected of having cancer or has been diagnosed with cancer. Cancers in which the cancer-testis nucleic acid or polypeptide are differentially expressed include testicular cancer, melanoma, small cell lung cancer, non-small cell lung cancer, colon cancer, renal cancer, bladder cancer and sarcoma. Additional cancers that can be diagnosed and/or treated using methods of the invention are described further below.

As used herein, a biological sample includes, but is not limited to: tissue, cells and/or body fluid (e.g. serum, blood, lymph node fluid, etc.). The fluid sample may include cells and/or fluid. The tissue and cells may be obtained from a subject or may be grown in culture (e.g. from a cell line). As used herein, a biological sample is body fluid, tissue or cells obtained from a subject using methods well-known to those of ordinary skill in the related medical arts. The biological sample preferably does not contain testis tissue.

The invention in another aspect permits the isolation of the cancer-associated antigens described herein. A variety of methodologies well-known to the skilled practitioner can be utilized to obtain isolated cancer-associated antigens. The proteins may be purified from cells which naturally produce the protein by chromatographic means or immunological recognition. Alternatively, an expression vector may be introduced into cells to cause production of the protein. In another method, mRNA transcripts may be microinjected or otherwise introduced into cells to cause production of the encoded protein. Translation of mRNA in cell-free extracts such as the reticulocyte lysate system also may be used to produce the protein. Those skilled in the art also can readily follow known methods for isolating cancer-associated antigens. These include, but are not limited to, chromatographic techniques such as immunochromatography, HPLC, size-exclusion chromatography, ion-exchange chromatography, and immune-affinity chromatography.

The invention also involves the use of agents such as polypeptides that bind to cancer-testis antigens. Such agents can be used in methods of the invention including the diagnosis and/or treatment of cancer. Such binding agents can be used, for example, in screening assays to detect the presence or absence of cancer-testis antigens and can be used in quantitative binding assays to determine levels of expression in biological samples and cells. Such agents also may be used to inhibit the native activity of the cancer-testis polypeptides, for example, by binding to such polypeptides.

According to this aspect, the binding polypeptides bind to an isolated nucleic acid or protein of the invention, including unique fragments thereof. Preferably, the binding polypeptides bind to a cancer-testis polypeptide, or a unique fragment thereof.

In preferred embodiments, the binding polypeptide is an antibody or antibody fragment, more preferably, an Fab or F(ab)₂ fragment of an antibody. Typically, the fragment includes a CDR3 region that is selective for the cancer-testis antigen. Any of the various types of antibodies can be used for this purpose, including polyclonal antibodies, monoclonal antibodies, humanized antibodies, and chimeric antibodies.

Thus, the invention provides agents which bind to cancer-testis antigens encoded by cancer-testis nucleic acid molecules of the invention, and in certain embodiments preferably to unique fragments of the cancer-testis polypeptides. Such binding partners can be used in screening assays to detect the presence or absence of a cancer-testis antigen and in purification protocols to isolate such cancer-testis antigens. Likewise, such binding partners can be used to selectively target drugs, toxins or other molecules (including detectable diagnostic molecules) to cells which express cancer-testis antigens. In this manner, for example, cells present in solid or non-solid tumors which express cancer-testis proteins can be treated with cytotoxic compounds that are selective for the cancer-testis molecules (nucleic acids and/or antigens). Such binding agents also can be used to inhibit the native activity of the cancer-testis antigen, for example, to further characterize the functions of these molecules.

The antibodies of the present invention are prepared by any of a variety of methods, including administering a protein, fragments of a protein, cells expressing the protein or fragments thereof and the like to an animal to induce polyclonal antibodies. The present invention also provides methods of producing monoclonal antibodies to the cancer-testis molecules of the invention described herein. The production of monoclonal antibodies is performed according to techniques well known in the art. As detailed herein, such antibodies may be used for example to identify tissues expressing protein or to purify protein. Antibodies also may be coupled to specific labeling agents or imaging agents, including, but not limited to a molecule preferably selected from the group consisting of fluorescent, enzyme, radioactive, metallic, biotin, chemiluminescent, bioluminescent, chromophore, or colored, etc. In some aspects of the invention, a label may be a combination of the foregoing molecule types.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762, and 5,859,205.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv, and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies (e.g., ScFv), (single) domain antibodies, and other intracellular antibodies.

Thus, the invention involves polypeptides of numerous size and type that bind specifically to cancer-testis antigens. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties.

The cancer-testis antigens of the invention can be used to screen peptide libraries, including phage display libraries, to identify and select peptide binding partners of the cancer-testis antigens of the invention. Such molecules can be used, as described, for screening assays, for diagnostic assays, for purification protocols or for targeting drugs, toxins and/or labeling agents (e.g., radioisotopes, fluorescent molecules, etc.) to cells which express cancer-testis molecules such as cancer cells which have aberrant cancer-testis expression.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to the cancer-testis antigen. This process can be repeated through several cycles of reselection of phage that bind to the cancer-testis polypeptide. Repeated rounds lead to enrichment of phage bearing particular sequences. DNA sequence analysis can be conducted to identify the sequences of the expressed polypeptides. The minimal linear portion of the sequence that binds to the cancer-testis polypeptide can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the cancer-testis antigens.

As detailed herein, the foregoing antibodies and other binding molecules may be used to identify tissues with normal or aberrant expression of a cancer-testis antigen. Antibodies also may be coupled to specific diagnostic labeling agents for imaging of cells and tissues with normal or aberrant cancer-testis antigen expression or to therapeutically useful agents according to standard coupling procedures. As used herein, “therapeutically useful agents” include any therapeutic molecule which desirably is targeted selectively to a cell or tissue selectively with an aberrant cancer-testis expression.

Diagnostic agents for in vivo use include, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99, iodine-131 and indium-111, and nuclides for nuclear magnetic resonance such as fluorine and gadolinium. Other diagnostic agents useful in the invention will be apparent to one of ordinary skill in the art.

The antibodies of the present invention can also be used to therapeutically target cancer-testis antigens. In a preferred embodiment, antibodies can be used to target antigens expressed on the cell surface. These antibodies can be linked not only to a detectable marker but also an antitumor agent or an immunomodulator. Antitumor agents can include cytotoxic agents and agents that act on tumor neovasculature. Detectable markers include, for example, radioactive or fluorescent markers. Cytotoxic agents include cytotoxic radionuclides, chemical toxins and protein toxins.

The cytotoxic radionuclide or radiotherapeutic isotope preferably is an alpha-emitting isotope such as ²²⁵Ac, ²¹¹At, ²¹²Bi, ²¹³Bi, ²¹²Pb, ²²⁴Ra or ²²³Ra. Alternatively, the cytotoxic radionuclide may a beta-emitting isotope such as ¹⁸⁶Rh, ¹⁸⁸Rh, ¹⁷⁷Lu, ⁹⁰Y, ¹³¹I, ⁶⁷Cu, ⁶⁴Cu, ¹⁵³Sm or ¹⁶⁶Ho. Further, the cytotoxic radionuclide may emit Auger and low energy electrons and include the isotopes ¹²⁵I, ¹²³I or ⁷⁷Br.

Suitable chemical toxins or chemotherapeutic agents include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Other antineoplastic agents that may be conjugated to the antibodies of the present invention include dolastatins (U.S. Pat. Nos. 6,034,065 and 6,239,104) and derivatives thereof. Of particular interest is dolastatin 10 (dolavaline-valine-dolaisoleuine-dolaproine-dolaphenine) and the derivatives auristatin PHE (dolavaline-valine-dolaisoleuine-dolaproine-phenylalanine-methyl ester) (Pettit, Q. R. et al., Anticancer Drug Des. 13(4):243-277, 1998; Woyke, T. et al., Antimicrob. Agents Chemother. 45(12):3580-3584, 2001), and aurastatin E and the like. Toxins that are less preferred in the compositions and methods of the invention include poisonous lectins, plant toxins such as ricin, abrin, modeccin, botulina and diphtheria toxins. Of course, combinations of the various toxins could also be coupled to one antibody molecule thereby accommodating variable cytotoxicity. Other chemotherapeutic agents are known to those skilled in the art.

Agents that act on the tumor vasculature can include tubulin-binding agents such as combrestatin A4 (Griggs et al., Lancet Oncol. 2:82, 2001), angiostatin and endostatin (reviewed in Rosen, Oncologist 5:20, 2000, incorporated by reference herein) and interferon inducible protein 10 (U.S. Pat. No. 5,994,292). A number of antiangiogenic agents currently in clinical trials are also contemplated. Agents currently in clinical trials include: 2ME2, Angiostatin, Angiozyme, Anti-VEGF RhuMAb, Apra (CT-2584), Avicine, Benefin, BMS275291, Carboxyamidotriazole, CC4047, CC5013, CC7085, CDC801, CGP-41251 (PKC 412), CM101, Combretastatin A-4 Prodrug, EMD 121974, Endostatin, Flavopiridol, Genistein (GCP), Green Tea Extract, IM-862, ImmTher, Interferon alpha, Interleukin-12, Iressa (ZD1839), Marimastat, Metastat (Col-3), Neovastat, Octreotide, Paclitaxel, Penicillamine, Photofrin, Photopoint, PI-88, Prinomastat (AG-3340), PTK787 (ZK22584), RO317453, Solimastat, Squalamine, SU 101, SU 5416, SU-6668, Suradista (FCE 26644), Suramin (Metaret), Tetrathiomolybdate, Thalidomide, TNP-470 and Vitaxin. Additional antiangiogenic agents are described by Kerbel, J. Clin. Oncol. 19(18s):45s-51s, 2001, which is incorporated by reference herein. Immunomodulators suitable for conjugation to the antibodies include α-interferon, γ-interferon, and tumor necrosis factor alpha (TNFα).

The coupling of one or more toxin molecules to the antibody is envisioned to include many chemical mechanisms, for instance covalent binding, affinity binding, intercalation, coordinate binding, and complexation. The toxic compounds used to prepare the immunotoxins are attached to the antibodies or antigen-binding fragments thereof by standard protocols known in the art.

As described herein, the cancer-testis molecules and the antibodies and other binding molecules, as described herein, can be used for the diagnosis, determination of prognosis and treatment of disorders. When “disorder” is used herein, it refers to any pathological condition where the cancer-testis antigens are aberrantly expressed. An example of such a disorder is cancer. For human cancers, additional particular examples include, biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma, neurosarcoma, chondrosarcoma, Ewing sarcoma, malignant fibrous histocytoma, glioma, esophageal cancer, hepatoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; testicular cancer; thyroid cancer including thyroid adenocarcinoma and medullar carcinoma; transitional cancer and renal cancer including adenocarcinoma and Wilms tumor.

Conventional treatment for cancer may include, but is not limited to: surgical intervention, chemotherapy, radiotherapy, and adjuvant systemic therapies. In one aspect of the invention, treatment may include administering binding polypeptides such as antibodies that specifically bind to the cancer-testis antigen. These binding polypeptides can be optionally linked to one or more detectable markers, antitumor agents or immunomodulators as described above.

Cancer treatment, in another aspect of the invention, includes administering an antisense molecules or RNAi molecules to reduce expression level and/or function level of cancer-testis polypeptides of the invention in the subject in cancers where a cancer-testis molecule is up-regulated or otherwise aberrantly overexpressed. The use of RNA interference or “RNAi” involves the use of double-stranded RNA (dsRNA) to block gene expression. (see: Sui, G, et al, Proc Natl. Acad. Sci. U.S.A. 99:5515-5520, 2002). Methods of applying RNAi strategies in embodiments of the invention would be understood by one of ordinary skill in the art.

Methods in which small interfering RNA (siRNA) molecules are used to reduce the expression of cancer-testis polypeptides may be used. In one aspect, a cell is contacted with a siRNA molecule to produce RNA interference (RNAi) that reduces expression of one or more cancer-testis polypeptides. The siRNA molecule is directed against nucleic acids coding for the cancer-testis polypeptide (e.g. RNA transcripts including untranslated and translated regions). In a preferred aspect of the invention the cancer-testis polypeptide is CT45. In a further preferred aspect the cancer-testis polypeptide is a CT46 polypeptide. The expression level of the targeted cancer-testis polypeptide(s) can be determined using well known methods such as Western blotting for determining the level of protein expression and Northern blotting or RT-PCR for determining the level of mRNA transcript of the target gene.

As used herein, a “siRNA molecule” is a double stranded RNA molecule (dsRNA) consisting of a sense and an antisense strand or a single stranded molecule that has a dsRNA component, for example a section of the molecule that hybridizes to itself (e.g., a “hairpin” structure). The antisense strand of the siRNA molecule is a complement of the sense strand (Tuschl, T. et al., 1999, Genes & Dev., 13:3191-3197; Elbashir, S. M. et al., 2001, EMBO J., 20:6877-6888; incorporated herein by reference). In one embodiment the last nucleotide at the 3′ end of the antisense strand may be any nucleotide and is not required to be complementary to the region of the target gene. The siRNA molecule may be 19-23 nucleotides in length and form a hairpin structure. In one preferred embodiment the siRNA molecule includes a two nucleotide 3′ overhang on the sense strand. In a second preferred embodiment the two nucleotide overhang is thymidine-thymidine (TT). The siRNA molecule corresponds to at least a portion of a target gene. In one embodiment the siRNA molecule corresponds to a region selected from a cDNA target gene beginning between 50 to 100 nucleotides downstream of the start codon. In a preferred embodiment the first nucleotide of the siRNA molecule is a purine.

The siRNA molecules can be plasmid-based. In a preferred method, a nucleic acid sequence that encodes a cancer-testis polypeptide is amplified using the well known technique of polymerase chain reaction (PCR). The use of the entire polypeptide encoding sequence is not necessary; as is well known in the art, a portion of the polypeptide encoding sequence is sufficient for RNA interference. The PCR fragment is inserted into a vector using routine techniques well known to those of skill in the art. In one aspect the nucleotide encoding sequence is the coding sequence of CT45. In another preferred aspect the nucleotide encoding sequence is the coding sequence of CT46. Combinations of the foregoing can be expressed from a single vector or from multiple vectors introduced into cells.

In one aspect of the invention a mammalian vector comprising any of the nucleotide coding sequences of the invention is provided. The mammalian vectors include but are not limited to the pSUPER RNAi vectors (Brummelkamp, T. R. et al., 2002, Science, 296:550-553, incorporated herein by reference). In one embodiment a nucleotide coding sequence can be inserted into the mammalian vector using restriction sites, creating a stem-loop structure. In a second embodiment, the mammalian vector may comprise the polymerase-III H1-RNA gene promoter. The polymerase-III H1-RNA promoter produces a RNA transcript lacking a polyadenosine tail and has a well-defined start of transcription and a termination signal consisting of five thymidines (T5) in a row. The cleavage of the transcript at the termination site occurs after the second uridine and yields a transcript resembling the ends of synthetic siRNAs containing two 3′ overhanging T or U nucleotides. The antisense strand of the siRNA molecule hybridizes to the corresponding region of the mRNA of the target gene.

Preferred systems for mRNA expression in mammalian cells are those such as pSUPER RNAi system as described in Brummelkamp et al. (2002, Science, 296:550-553). Other examples include but are not limited to pSUPER.neo, pSUPER.neo+gfp, pSUPER.puro, BLOCK-iT T7-TOPO linker, pcDNA1.2/V5-GW/lacZ, pENTRJU6, pLenti6-GW/U6-laminshrna, and pLenti6/BLOCK-iT-DEST. These vectors are available from suppliers such as Invitrogen, and one of skill in the art would be able to obtain and use them.

Cancer-testis polypeptides as described herein, can also be used in one aspect of the invention to induce or enhance an immune response. Some therapeutic approaches based upon the disclosure are premised on a response by a subject's immune system, leading to lysis of antigen presenting cells, such as cancer cells which present one or more cancer-testis antigens of the invention. One such approach is the administration of autologous CTLs specific to a cancer-testis antigen/MHC complex to a subject with abnormal cells of the phenotype at issue. It is within the ability of one of ordinary skill in the art to develop such CTLs in vitro. An example of a method for T cell differentiation is presented in International Application number PCT/US96/05607. Generally, a sample of cells taken from a subject, such as blood cells, are contacted with a cell presenting the complex and capable of provoking CTLs to proliferate. The target cell can be a transfectant, such as a COS cell. Alternatively, instead of transfecting COS cells, one might use autologous APCs such as dendritic cells (DCs) purified from PBMC. DCs could be transfected or pulsed with antigen, either full length protein or peptide antigens. (Ayyoub, M et al J. Immunol. 2004 172:7206-7211, Ayyoub M. et al. J Clin Invest 2004 113:1225-33.) These transfectants present the desired complex of their surface and, when combined with a CTL of interest, stimulate its proliferation. COS cells are widely available, as are other suitable host cells. Specific production of CTL clones is well known in the art. The clonally expanded autologous CTLs then are administered to the subject.

Another method for selecting antigen-specific CTL clones has been described (Altman et al., Science 274:94-96, 1996; Dunbar et al. Curr. Biol. 8:413-416, 1998), in which fluorogenic tetramers or multimers of MHC class I molecule/peptide complexes are used to detect specific CTL clones. Briefly, soluble MHC class I molecules are folded in vitro in the presence of β₂-microglobulin and a peptide antigen which binds the class I molecule. After purification, the MHC/peptide complex is purified and labeled with biotin. Tetramers are formed by mixing the biotinylated peptide-MHC complex with labeled avidin (e.g. phycoerythrin) at a molar ratio or 4:1. Tetramers are then contacted with a source of CTLs such as peripheral blood or lymph node. The tetramers bind CTLs which recognize the peptide antigen/MHC class I complex. Cells bound by the tetramers can be sorted by fluorescence activated cell sorting to isolate the reactive CTLs. The isolated CTLs then can be expanded in vitro for use as described herein. The use of MHC class II molecules as tetramers was recently demonstrated by Crawford et al. (Immunity 8:675-682, 1998; see also Dunbar and Ogg, J. Immunol. Methods 268(1):3-7, 2002; Arnold et al., J. Immunol. Methods 271(1-2):137-151, 2002). Multimeric soluble MHC class II molecules were complexed with a covalently attached peptide (which can be attached with or without a linker molecule), but peptides also can be loaded onto class II molecules. The class II tetramers were shown to bind with appropriate specificity and affinity to specific T cells. Thus tetramers can be used to monitor both CD4⁺ and CD8⁺ cell responses to vaccination protocols. Methods for preparation of multimeric complexes of MHC class II molecules are described in Hugues et al., J. Immunological Meth. 268: 83-92 (2002) and references cited therein, each of which is incorporated by reference.

Computational methods for selecting amino acid substitutions, such as iterative computer structural modeling, can also be performed by one of ordinary skill in the art to prepare variants. HLA class II binding peptide functional variants can be developed by analysis of the binding domains or binding pockets of major histocompatibility complex HLA-DR proteins and/or the T cell receptor (“TCR”) contact points of HLA class II binding peptides. By providing a detailed structural analysis of the residues involved in forming the HLA class II binding pockets, one is enabled to make predictions of sequence motifs for binding of peptides to any of the HLA class II proteins.

Using these sequence motifs as search, evaluation, or design criteria, one is enabled to identify classes of peptides which have a reasonable likelihood of binding to a particular HLA molecule and of interacting with a T cell receptor to induce T cell response. These peptides can be synthesized and tested for activity as described herein. Use of these motifs, as opposed to pure sequence homology (which excludes many peptides which are antigenically similar but quite distinct in, sequence) or sequence homology with unlimited “conservative” substitutions (which admits many peptides which differ at critical highly conserved sites), represents a method by which one of ordinary skill in the art can evaluate peptides for potential application in the treatment of disease.

The Strominger and Wucherpfennig PCT application (PCT/US96/03182), and references cited therein, all of which are incorporated by reference, describe the HLA class II and TCR binding pockets which contact residues of an HLA class II peptide. By keeping the residues which are likely to bind in the HLA class II and/or TCR binding pockets constant or permitting only specified substitutions, functional variants of HLA class II binding peptides can be prepared which retain binding to HLA class II and T cell receptor.

In one therapeutic methodology, referred to as adoptive transfer (Greenberg, J. Immunol. 136(5): 1917, 1986; Riddel et al., Science 257: 238, 1992; Lynch et al, Eur. J. Immunol. 21: 1403-1410, 1991; Kast et al., Cell 59: 603-614, 1989), cells presenting the desired complex (e.g., dendritic cells) are combined with CTLs leading to proliferation of the CTLs specific thereto. The proliferated CTLs are then administered to a subject with a cellular abnormality which is characterized by certain of the abnormal cells presenting the particular complex. The CTLs then lyse the abnormal cells, thereby achieving the desired therapeutic goal.

The foregoing therapy assumes that at least some of the subject's abnormal cells present the relevant HLA/cancer associated antigen complex. This can be determined very easily, as the art is very familiar with methods for identifying cells which present a particular HLA molecule, as well as how to identify cells expressing DNA of the pertinent sequences, in this case a cancer-testis antigen sequence. Once cells presenting the relevant complex are identified via the foregoing screening methodology, they can be combined with a sample from a patient, where the sample contains CTLs. If the complex presenting cells are lysed by the mixed CTL sample, then it can be assumed that a cancer-testis antigen is being presented, and the subject is an appropriate candidate for the therapeutic approaches set forth supra.

Adoptive transfer is not the only form of therapy that is available in accordance with the invention. CTLs can also be provoked in vivo, using a number of approaches. One approach is the use of non-proliferative cells expressing the complex. The cells used in this approach may be those that normally express the complex, such as irradiated tumor cells or cells transfected with one or both of the genes necessary for presentation of the complex (i.e. the antigenic peptide and the presenting MHC molecule). Chen et al. (Proc. Natl. Acad. Sci. USA 88: 110-114, 1991) exemplifies this approach, showing the use of transfected cells expressing HPV E7 peptides in a therapeutic regime. Various cell types may be used. Similarly, vectors carrying one or both of the genes of interest may be used. Viral or bacterial vectors are especially preferred. For example, nucleic acids which encode a cancer-testis polypeptide may be operably linked to promoter and enhancer sequences which direct expression of the cancer-testis antigen polypeptide in certain tissues or cell types. The nucleic acid may be incorporated into an expression vector.

Expression vectors may be unmodified extrachromosomal nucleic acids, plasmids or viral genomes constructed or modified to enable insertion of exogenous nucleic acids, such as those encoding cancer-testis antigen, as described elsewhere herein. Nucleic acids encoding a cancer-testis antigen also may be inserted into a retroviral genome, thereby facilitating integration of the nucleic acid into the genome of the target tissue or cell type. In these systems, the gene of interest is carried by a microorganism, e.g., a Vaccinia virus, pox virus, herpes simplex virus, retrovirus or adenovirus, and the materials de facto “infect” host cells. The cells which result present the complex of interest, and are recognized by autologous CTLs, which then proliferate.

A similar effect can be achieved by combining the cancer-testis polypeptide or a stimulatory fragment thereof with an adjuvant to facilitate incorporation into antigen presenting cells in vivo. The cancer-testis polypeptide is processed to yield the peptide partner of the MHC molecule while a cancer-testis fragment may be presented without the need for further processing. Generally, subjects can receive an intradermal, intravenous, subcutaneous or intramuscular injection of an effective amount of the cancer-testis antigen. Initial doses can be followed by bi- or tri-weekly, weekly or monthly booster doses, following immunization protocols standard in the art. Preferred cancer-testis antigens include those where evidence of naturally or spontaneously induced immunity can be observed. This might be the demonstration of antigen-specific CD8 or CD4 T cells in a high frequency of cancer patients with antigen expressing tumors or the presence of autologous antigen-specific antibodies in such cancer patients, preferably both (Jager et al. PNAS 2000 97:4700-5; Gnjatic et al PNAS 2003 100:8862-7).

The invention involves the use of various materials disclosed herein to “immunize” subjects or as “vaccines”. As used herein, “immunization” or “vaccination” means increasing or activating an immune response against an antigen. It does not require elimination or eradication of a condition but rather contemplates the clinically favorable enhancement of an immune response toward an antigen. Generally accepted animal models can be used for testing of immunization against cancer using a cancer-testis nucleic acid. For example, human cancer cells can be introduced into a mouse to create a tumor, and one or more cancer-testis nucleic acids can be delivered by the methods described herein. The effect on the cancer cells (e.g., reduction of tumor size) can be assessed as a measure of the effectiveness of the cancer-testis nucleic acid immunization. Of course, testing of the foregoing animal model using more conventional methods for immunization include the administration of one or more cancer-testis polypeptides or fragments derived therefrom, optionally combined with one or more adjuvants and/or cytokines to boost the immune response.

Methods for immunization, including formulation of a vaccine composition and selection of doses, route of administration and the schedule of administration (e.g. primary and one or more booster doses), are well known in the art. The tests also can be performed in humans, where the end point is to test for the presence of enhanced levels of circulating CTLs against cells bearing the antigen, to test for levels of circulating antibodies against the antigen, to test for the presence of cells expressing the antigen and so forth.

As part of the immunization compositions, one or more cancer-tests polypeptides or immunogenic fragments thereof are administered with one or more adjuvants to induce an immune response or to increase an immune response. An adjuvant is a substance incorporated into or administered with antigen which potentiates the immune response. Adjuvants may enhance the immunological response by providing a reservoir of antigen (extracellularly or within macrophages), activating macrophages and stimulating specific sets of lymphocytes. Adjuvants of many kinds are well known in the art. Specific examples of adjuvants include monophosphoryl lipid A (MPL, SmithKline Beecham), a congener obtained after purification and acid hydrolysis of Salmonella minnesota Re 595 lipopolysaccharide; saponins including QS21 (SmithKline Beecham), a pure QA-21 saponin purified from Quillja saponaria extract; DQS21, described in PCT application WO96/33739 (SmithKline Beecham), ISCOM (CSL Ltd., Parkville, Victoria, Australia) derived from the bark of the Quillaia saponaria molina tree; QS-7, QS-17, QS-18, and QS-L1 (So et al., Mol. Cells. 7:178-186, 1997); incomplete Freund's adjuvant; complete Freund's adjuvant; montanide; alum; CpG oligonucleotides (see e.g. Kreig et al., Nature 374:546-9, 1995; U.S. Pat. No. 6,207,646) and other immunostimulatory oligonucleotides; various water-in-oil emulsions prepared from biodegradable oils such as squalene and/or tocopherol; and factors that are taken up by the so-called ‘toll-like receptor 7’ on certain immune cells that are found in the outside part of the skin, such as imiquimod (3M, St. Paul, Minn.). Preferably, the antigens are administered mixed with a combination of DQS21/MPL. The ratio of DQS21 to MPL typically will be about 1:10 to 10:1, preferably about 1:5 to 5:1 and more preferably about 1:1. Typically for human administration, DQS21 and MPL will be present in a vaccine formulation in the range of about 1 μg to about 100 μg. Other adjuvants are known in the art and can be used in the invention (see, e.g. Goding, Monoclonal Antibodies: Principles and Practice, 2nd Ed., 1986). Methods for the preparation of mixtures or emulsions of polypeptide and adjuvant are well known to those of skill in the art of vaccination.

Other agents which stimulate the immune response of the subject can also be administered to the subject. For example, other cytokines are also useful in vaccination protocols as a result of their lymphocyte regulatory properties. Many other cytokines useful for such purposes will be known to one of ordinary skill in the art, including interleukin-12 (IL-12) which has been shown to enhance the protective effects of vaccines (see, e.g., Science 268: 1432-1434, 1995), GM-CSF, IL-18 and IL-15 (Klebanoff et al. Proc. Natl. Acad. Sci. USA 2004 101:1969-74). Thus cytokines can be administered in conjunclion with antigens and adjuvants to increase the immune response to the antigens.

There are a number of immune response potentiating compounds that can be used in vaccination protocols. These include costimulatory molecules provided in either protein or nucleic acid form. Such costimulatory molecules include the B7-1 and B7-2 (CD80 and CD86 respectively) molecules which are expressed on dendritic cells (DC) and interact with the CD28 molecule expressed on the T cell. This interaction provides costimulation (signal 2) to an antigen/MHC/TCR stimulated (signal 1) T cell, increasing T cell proliferation and effector function. B7 also interacts with CTLA4 (CD152) on T cells and studies involving CTLA4 and B7 ligands indicate that the B7-CTLA4 interaction can enhance antitumor immunity and CTL proliferation (Zheng P., et al. Proc. Natl. Acad. Sci. USA 95 (11):6284-6289 (1998)).

B7 typically is not expressed on tumor cells so they are not efficient antigen presenting cells (APCs) for T cells. Induction of B7 expression would enable the tumor cells to stimulate more efficiently CTL proliferation and effector function. A combination of B7/IL-6/IL-12 costimulation has been shown to induce IFN-gamma and a Th1 cytokine profile in the T cell population leading to further enhanced T cell activity (Gajewski et al., J. Immunol, 154:5637-5648 (1995)). Tumor cell transfection with B7 has been discussed in relation to in vitro CTL expansion for adoptive transfer immunotherapy by Wang et al., (J. Immunol., 19:1-8 (1986)). Other delivery mechanisms for the B7 molecule would include nucleic acid (naked DNA) immunization (Kim J., et al. Nat. Biotechnol., 15:7:641-646 (1997)) and recombinant viruses such as adeno and pox (Wendtner et al., Gene Ther., 4:7:726-735 (1997)). These systems are all amenable to the construction and use of expression cassettes for the coexpression of B7 with other molecules of choice such as the antigens or fragment(s) of antigens discussed herein (including polytopes) or cytokines.

These delivery systems can be used for induction of the appropriate molecules in vitro and for in vivo vaccination situations. The use of anti-CD28 antibodies to directly stimulate T cells in vitro and in vivo could also be considered. Similarly, the inducible co-stimulatory molecule ICOS which induces T cell responses to foreign antigen could be modulated, for example, by use of anti-ICOS antibodies (Hutloff et al., Nature 397:263-266, 1999).

Lymphocyte function associated antigen-3 (LFA-3) is expressed on APCs and some tumor cells and interacts with CD2 expressed on T cells. This interaction induces T cell IL-2 and IFN-gamma production and can thus complement but not substitute, the B7/CD28 costimulatory interaction (Parra et al., J. Immunol., 158:637-642 (1997), Fenton et al., J. Immunother., 21:2:95-108 (1998)).

Lymphocyte function associated antigen-1 (LFA-1) is expressed on leukocytes and interacts with ICAM-1 expressed on APCs and some tumor cells. This interaction induces T cell IL-2 and IFN-gamma production and can thus complement but not substitute, the B7/CD28 costimulatory interaction (Fenton et al., J. Immunother., 21:2:95-108 (1998)). LFA-1 is thus a further example of a costimulatory molecule that could be provided in a vaccination protocol in the various ways discussed above for B7.

Complete CTL activation and effector function requires Th cell help through the interaction between the Th cell CD40L (CD40 ligand) molecule and the CD40 molecule expressed by DCs (Ridge et al., Nature, 393:474 (1998), Bennett et al., Nature, 393:478 (1998), Schoenberger et al., Nature, 393:480 (1998)). This mechanism of this costimulatory signal is likely to involve upregulation of B7 and associated IL-6/IL-12 production by the DC (APC). The CD40-CD40L interaction thus complements the signal 1 (antigen/MHC-TCR) and signal 2 (B7-CD28) interactions.

The use of anti-CD40 antibodies to stimulate DC cells directly, would be expected to enhance a response to tumor antigens which are normally encountered outside of an inflammatory context or are presented by non-professional APCs (tumor cells). In these situations Th help and B7 costimulation signals are not provided.

The invention contemplates delivery of nucleic acids, polypeptides or fragments thereof for vaccination. Delivery of polypeptides and fragments thereof can be accomplished according to standard vaccination protocols which are well known in the art. In another embodiment, the delivery of nucleic acid is accomplished by ex vivo methods, i.e. by removing a cell from a subject, genetically engineering the cell to include a cancer-testis polypeptide, and reintroducing the engineered cell into the subject. One example of such a procedure is outlined in U.S. Pat. No. 5,399,346 and in exhibits submitted in the file history of that patent, all of which are publicly available documents. In general, it involves introduction in vitro of a functional copy of a gene into a cell(s) of a subject, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo nucleic acid delivery using vectors such as viruses and targeted liposomes also is contemplated according to the invention.

A virus vector for delivering a nucleic acid encoding a cancer-testis polypeptide is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol 26:1951-1959, 1996). A preferred virus vector is an adenovirus.

Preferably the foregoing nucleic acid delivery vectors: (1) contain exogenous genetic material that can be transcribed and translated in a mammalian cell and that can induce an immune response in a host, and (2) contain on a surface a ligand that selectively binds to a receptor on the surface of a target cell, such as a mammalian cell, and thereby gains entry to the target cell.

Various techniques may be employed for introducing nucleic acids of the invention into cells, depending on whether the nucleic acids are introduced in vitro or in vivo in a host. Such techniques include transfection of nucleic acid-CaPO₄ precipitates, transfection of nucleic acids associated with DEAE, transfection or infection with the foregoing viruses including the nucleic acid of interest, liposome mediated transfection, and the like. For certain uses, it is preferred to target the nucleic acid to particular cells. In such instances, a vehicle used for delivering a nucleic acid of the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. Preferred antibodies include antibodies which selectively bind a cancer-testis antigen, alone or as a complex with a MHC molecule. Especially preferred are monoclonal antibodies. Where liposomes are employed to deliver the nucleic acids of the invention, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

According to a further aspect of the invention, compositions containing the nucleic acid molecules, proteins, and binding polypeptides of the invention are provided. The compositions contain any of the foregoing nucleic acid molecules, proteins, and binding polypeptides (as therapeutic agents) in an optional pharmaceutically acceptable carrier. Thus, in a related aspect, the invention provides a method for forming a medicament that involves placing a therapeutically effective amount of the therapeutic agent in the pharmaceutically acceptable carrier to form one or more doses. The effectiveness of treatment or prevention methods of the invention can be determined using standard diagnostic methods described herein.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The therapeutics of the invention can be administered by any conventional route, including injection or by gradual infusion over time. The administration may, for example, be oral, intravenous, intratumoral, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712). Those of skill in the art can readily determine the various parameters and conditions for producing antibody aerosols without undue experimentation. When using antisense preparations of the invention, slow intravenous administration is preferred.

The compositions of the invention are administered in effective amounts. An “effective amount” is that amount of a cancer-testis polypeptide composition that alone, or together with further doses, produces the desired response, e.g. increases an immune response to the cancer-testis polypeptide. In the case of treating a particular disease or condition characterized by expression of one or more cancer-testis polypeptides, such as cancer, the desired response is inhibiting the progression of the disease. This may involve only slowing the progression of the disease temporarily, although more preferably, it involves halting the progression of the disease permanently. This can be monitored by routine methods or can be monitored according to diagnostic methods of the invention discussed herein. The desired response to treatment of the disease or condition also can be delaying the onset or even preventing the onset of the disease or condition.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of cancer-testis polypeptide or nucleic acid encoding cancer-testis polypeptide for producing the desired response in a unit of weight or volume suitable for administration to a patient. The response can, for example, be measured by determining the immune response following administration of the cancer-testis polypeptide composition via a reporter system by measuring downstream effects such as gene expression, or by measuring the physiological effects of the cancer-testis polypeptide composition, such as regression of a tumor or decrease of disease symptoms. Other assays will be known to one of ordinary skill in the art and can be employed for measuring the level of the response.

The doses of cancer-testis polypeptide compositions (e.g., polypeptide, peptide, antibody, cell or nucleic acid) administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, for treatments for eliciting or increasing an immune response, doses of cancer-testis antigen are formulated and administered in doses between 1 ng and 1 mg, and preferably between 10 ng and 100 μg, according to any standard procedure in the art. Where nucleic acids encoding cancer-testis polypeptides or variants thereof are employed, doses of between 1 ng and 0.1 mg generally will be formulated and administered according to standard procedures. Other protocols for the administration of cancer-testis polypeptide compositions will be known to one of ordinary skill in the art, in which the dose amount, schedule of injections, sites of injections, mode of administration (e.g., intra-tumoral) and the like vary from the foregoing. Administration of cancer-testis polypeptide compositions to mammals other than humans, e.g. for testing purposes or veterinary therapeutic purposes, is carried out under substantially the same conditions as described above.

Where cancer-testis polypeptides are used for vaccination, modes of administration which effectively deliver the cancer-testis polypeptide and adjuvant, such that an immune response to the polypeptide is increased, can be used. For administration of a cancer-testis polypeptide in adjuvant, preferred methods include intradermal, intravenous, intramuscular and subcutaneous administration. Although these are preferred embodiments, the invention is not limited by the particular modes of administration disclosed herein. Standard references in the art (e.g., Remington's Pharmaceutical Sciences, 18th edition, 1990) provide modes of administration and formulations for delivery of immunogens with adjuvant or in a non-adjuvant carrier.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, and lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases, and the like.

The pharmaceutical agents of the invention may be administered alone, in combination with each other, and/or in combination with other anti-cancer drug therapies and/or treatments. These therapies and/or treatments may include, but are not limited to: surgical intervention, chemotherapy, radiotherapy, and adjuvant systemic therapies.

The invention also provides a pharmaceutical kit comprising one or more containers comprising one or more of the pharmaceutical compounds or agents of the invention. Additional materials may be included in any or all kits of the invention, and such materials may include, but are not limited to buffers, water, enzymes, tubes, control molecules, etc. The kit may also include instructions for the use of the one or more pharmaceutical compounds or agents of the invention for the treatment of cancer.

The invention further includes nucleic acid or protein microarrays (including antibody arrays) for the analysis of expression of cancer-testis antigens or nucleic acids encoding such antigens. In this aspect of the invention, standard techniques of microarray technology are utilized to assess expression of the cancer-testis antigens and/or identify biological constituents that bind such antigens. The constituents of biological samples include antibodies, lymphocytes (particularly T lymphocytes), and the like. Microarray substrates include but are not limited to glass, silica, aluminosilicates, borosilicates, metal oxides such as alumina and nickel oxide, various clays, nitrocellulose, or nylon. The microarray substrates may be coated with a compound to enhance synthesis of a probe (peptide or nucleic acid) on the substrate. Coupling agents or groups on the substrate can be used to covalently link the first nucleotide or amino acid to the substrate. A variety of coupling agents or groups are known to those of skill in the art. Peptide or nucleic acid probes thus can be synthesized directly on the substrate in a predetermined grid. Alternatively, peptide or nucleic acid probes can be spotted on the substrate, and in such cases the substrate may be coated with a compound to enhance binding of the probe to the substrate. In these embodiments, presynthesized probes are applied to the substrate in a precise, predetermined volume and grid pattern, preferably utilizing a computer-controlled robot to apply probe to the substrate in a contact-printing manner or in a non-contact manner such as ink jet or piezo-electric delivery. Probes may be covalently linked to the substrate. Nucleic acid probes preferably are linked using UV irradiation or heat.

Protein microarray technology, which is also known by other names including protein chip technology and solid-phase protein array technology, is well known to those of ordinary skill in the art and is based on, but not limited to, obtaining an array of identified peptides or proteins on a fixed substrate, binding target molecules or biological constituents to the peptides, and evaluating such binding. See, e.g., G. MacBeath and S. L. Schreiber, “Printing Proteins as Microarrays for High-Throughput Function Determination,” Science 289(5485):1760-1763, 2000.

Targets are peptides or proteins and may be natural or synthetic. The tissue may be obtained from a subject or may be grown in culture (e.g. from a cell line).

In some embodiments of the invention, one or more control peptide or protein molecules are attached to the substrate. Preferably, control peptide or protein molecules allow determination of factors such as peptide or protein quality and binding characteristics, reagent quality and effectiveness, hybridization success, and analysis thresholds and success.

Nucleic acid arrays, particularly arrays that bind nucleic acids encoding cancer-testis antigens, also can be used for diagnostic applications, such as for identifying subjects that have a condition characterized by aberrant cancer-testis antigen expression. Nucleic acid microarray technology, which is also known by other names including: DNA chip technology, gene chip technology, and solid-phase nucleic acid array technology, is well known to those of ordinary skill in the art and is based on, but not limited to, obtaining an array of identified nucleic acid probes on a fixed substrate, labeling target molecules with reporter molecules (e.g., radioactive, chemiluminescent, or fluorescent tags such as fluorescein, Cye3-dUTP, or Cye5-dUTP), hybridizing target nucleic acids to the probes, and evaluating target-probe hybridization. A probe with a nucleic acid sequence that perfectly matches the target sequence will, in general, result in detection of a stronger reporter-molecule signal than will probes with less perfect matches. Many components and techniques utilized in nucleic acid microarray technology are presented in The Chipping Forecast, Nature Genetics, Vol. 21, January 1999, the entire contents of which is incorporated by reference herein.

According to the invention, probes are selected from the group of nucleic acids including, but not limited to: DNA, genomic DNA, cDNA, and oligonucleotides; and may be natural or synthetic. Oligonucleotide probes preferably are 20 to 25-mer oligonucleotides and DNA/cDNA probes preferably are 500 to 5000 bases in length, although other lengths may be used. Appropriate probe length may be determined by one of ordinary skill in the art by following art-known procedures. In one embodiment, preferred probes are sets of one or more of the cancer-testis nucleic acid molecules as described herein. Probes may be purified to remove contaminants using standard methods known to those of ordinary skill in the art such as gel filtration or precipitation.

In one embodiment, the microarray substrate may be coated with a compound to enhance synthesis of the probe on the substrate. Such compounds include, but are not limited to, oligoethylene glycols. In another embodiment, coupling agents or groups on the substrate can be used to covalently link the first nucleotide or oligonucleotide to the substrate. These agents or groups may include, for example, amino, hydroxy, bromo, and carboxy groups. These reactive groups are preferably attached to the substrate through a hydrocarbyl radical such as an alkylene or phenylene divalent radical, one valence position occupied by the chain bonding and the remaining attached to the reactive groups. These hydrocarbyl groups may contain up to about ten carbon atoms, preferably up to about six carbon atoms. Alkylene radicals are usually preferred containing two to four carbon atoms in the principal chain. These and additional details of the process are disclosed, for example, in U.S. Pat. No. 4,458,066, which is incorporated by reference in its entirety.

In one embodiment, nucleic acid probes are synthesized directly on the substrate in a predetermined grid pattern using methods such as light-directed chemical synthesis, photochemical deprotection, or delivery of nucleotide precursors to the substrate and subsequent probe production.

Targets for microarrays are nucleic acids selected from the group, including but not limited to: DNA, genomic DNA, cDNA, RNA, mRNA and may be natural or synthetic. In all embodiments, nucleic acid target molecules from human tissue are preferred. The tissue may be obtained from a subject or may be grown in culture (e.g. from a cell line).

In embodiments of the invention one or more control nucleic acid molecules are attached to the substrate. Preferably, control nucleic acid molecules allow determination of factors such as nucleic acid quality and binding characteristics, reagent quality and effectiveness, hybridization success, and analysis thresholds and success. Control nucleic acids may include but are not limited to expression products of genes such as housekeeping genes or fragments thereof.

### Example 1

### CT45

### Materials and Methods

Tumor Tissues and Cell Lines. Specimens of tumor tissues were obtained from Departments of Pathology at the Weill Medical College of Cornell University and Memorial Sloan-Kettering Cancer Center. Cell lines were obtained from the cell line bank maintained by the Ludwig Institute for Cancer Research, New York Branch, New York, N.Y.
MPSS. Pooled normal human tissue RNA preparations were purchased from Clontech (Palo Alto, Calif.). In addition, mRNA was purified from two cancer cell lines, SK-MEL-37 and SK-LU-17, using standard protocols. After DNase treatment and isolation of poly(A)+ RNA, these samples were used to generate cDNA libraries according to the Megaclone protocol (Brenner, S., Williams, et al., (2000), Proc Natl Acad Sci U.S.A., 97:1665-70), and signature sequences adjacent to poly(A) proximal DpnII restriction sites were obtained by serial cutting and ligation of decoding adapters (Brenner, S., Johnson, et al., (2000), Nat. Biotechnol., 18:630-4). Each signature comprised 17 nucleotides, including the DpnII recognition sequence (GATC). Between 2 million and 3 million tags were sequenced from each sample, in two reading frames offset by two nucleotides. Only signatures that were seen in two independent sequencing runs and present at a minimum of 5 transcripts per million in at least one sample were retained for the analysis.

The mapping of signatures to human transcripts was performed essentially as described before (Jongeneel, C. V. et al., (2003), Proc. Natl. Acad. Sci. U.S.A., 100:4702-5), using the National Center for Biotechnology Information (NCBI) assembly 33 of the human genome. Sequence polymorphisms present in EST sequences but not in the genomic reference sequence were taken into account for the mapping. Signatures that unambiguously matched transcribed regions were retained. Counts were pooled when multiple signatures mapped to the same gene.

In silico analysis. To identify candidate CT genes from the list of 1056 MPSS-defined testis-specific genes, the expression profile of each gene in normal and tumor tissues were evaluated using a combination of the SAGE Anatomic Viewer and its Virtual Northern tool (refer to The Cancer Genome Anatomy Project CGAP website for SAGE and Anatomic viewer: cgap.nci.nih.gov/SAGE/AnatomicViewer), and database searches using BLASTN (refer to The National Library of Medicine website for BLAST: ncbi.nlm.nih.gov/BLAST). The focus of the analysis was to identify Unigene clusters containing ESTs derived from testis as well as from non-germ cell tumors and with limited expression in somatic tissues. Once a Unigene cluster was considered to be a likely CT candidate, the intron-exon structure of the corresponding gene was defined using the tools on the NCBI Web site (refer to The National Library of Medicine website: ncbi.nlm.nih.gov). This information was then used to design trans-intronic primers for RT-PCR.

For some genes, e.g. CT45 (see below), the NCBI Web site (ncbi.nlm.nih.gov) was used for protein similarity searches, the identification of conserved domains, chromosomal localization, the location of DNA contigs, and transcripts/proteins prediction. The MyHits database (refer to the Swiss Institute of Bioinformatics website: myhits.isb-sib.ch) was used to explore potential protein domains. Gene identifiers were retrieved from the Ensembl database (refer to The Wellcome Trust Sanger Institute website: ensembl.org), to maintain a consistent naming convention, and short names were assigned to each previously uncharacterized gene identified in the project, using Human Gene Nomenclature Committee (HGNC)—approved symbols whenever possible.

Qualitative RT-PCR. A normalized cDNA panel was used that comprises brain, colon, heart, kidney, leukocytes, liver, lung, ovary, pancreas, placenta, prostate, skeletal muscle, small intestine, spleen, thymus, and testis (MTC panels I and II, BD Biosciences, Franklin Lakes, N.J.). For evaluating the expression in tumor cell lines, RNA was prepared by the standard guanidinium thiocyanate-CsCl gradient method. Total RNA (2 μg) was used for 20 μl reverse-transcriptase reaction, and 2 μl of cDNA was used per 25 μl PCR. PCR was performed using Invitrogen Platinum Taq Supermix with 35 cycles each consisting of 15 sec at 94° C., 1 min at 60° C., and 1 min at 72° C. PCR products were visualized on 1% agarose gel electrophoresis by ethidium bromide staining.
Quantitative RT-PCR. Quantitative RT-PCR was performed using PRISM 7000 sequence detection system (Applied Biosystems, Foster City, Calif.). Normal testis RNA was obtained from Ambion (Austin, Tex.). RNA from tumor tissue was prepared by using TriZol reagents (Life Technologies; Carlsbad, Calif.). Two micrograms of total RNA was used per 20 μl reverse transcription reaction, and 2 μl of cDNA was used for each 25 μl PCR. Reactions were in duplicate, and the level of expression was determined relative to the testicular preparation. A standard curve was established for each PCR plate by using testicular cDNA in 4-fold serial dilutions. Forty-five two-step cycles amplification were undertaken, each cycle consisting of 15 sec at 95° C. and 1 min at 60° C. The RNA quality of the cell lines and tissues was evaluated by amplification of β-glucuromidase (GUS) and GAPDH. All specimens included in the final analysis had cycle time (Ct) values differing by fewer than four cycles, indicating similar qualities and quantities of the cDNA used.

### Results

Identification of candidate CT genes. MPSS data were obtained from 32 normal tissues, including two separate preparations of testis and placenta and two CT-rich cell lines, SK-MEL-37 and SK-LC-17. Genes were considered to have testis-predominant expression when the number of corresponding MPSS tags in the testis was at least two times greater than the combined number of tags in all somatic tissues. A total of 1056 such testis-predominant genes were identified (Table 1), of which thirty-nine are located on chromosome X; a chromosome known to contain many CT antigen genes (Scanlan, M. J. et at, (2004), Cancer Immun., 4:1). Nine of these 39 genes encode known CT antigens, NY-ESO-1, LAGE1, CT10, MAGE-B1, -B2, and -B4, GAGE1, GAGE2, and PAGE5, demonstrating that this approach can potentially identify new genes encoding CT antigens. Other CT antigen-encoding genes in the 1,056 gene list included SCP1 (chromosome 1), CT9/BRDT (chromosome 1), OY-TES-1/ACRBP (chromosome 12), ADAM2 (chromosome 8), ADAM21 (chromosome 14), and TPTE (chromosome 21).

TABLE 1

| | Testis-specific genes | Genes tested for normal and cell |
|---|---|---|
| Chromosome | (MPSS tag in testis >10) | lines |
| X | 39 | 2 |
| Y | 6 | 0 |
| 1 | 93 | 6 |
| 2 | 75 | 7 |
| 3 | 60 | 11 |
| 4 | 50 | 3 |
| 5 | 53 | 1 |
| 6 | 63 | 5 |
| 7 | 51 | 2 |
| 8 | 36 | 1 |
| 9 | 46 | 4 |
| 10 | 38 | 2 |
| 11 | 61 | 4 |
| 12 | 38 | 0 |
| 13 | 28 | 0 |
| 14 | 31 | 2 |
| 15 | 48 | 4 |
| 16 | 44 | 3 |
| 17 | 42 | 4 |
| 18 | 24 | 0 |
| 19 | 53 | 7 |
| 20 | 42 | 3 |
| 21 | 7 | 0 |
| 22 | 28 | 0 |
| Total | 1056 | 71 |

The 1,041 genes that did not correspond to known CT genes were analyzed by using the MPSS data from SK-MEL-37 and SK-LC-17, as well as ESTs from the public database. Candidate CT genes were taken as those with ESTs or MPSS tags from cancer tissues or cell lines (excluding germ cell or testicular tumors), and where ESTs were not found in more than two normal somatic tissues, excluding fetal tissues and pooled tissues. Pooled tissues were excluded because they often include testis, and fetal tissue was excluded because its capacity to express CT antigens has yet to be determined.

Based on these criteria, 202 genes were identified, of which 36 were found to be intronless genes and were excluded from further analysis. Trans-intronic primers were designed for the remaining 166 genes.

mRNA Expression of CT-candidate Genes in Normal Tissues and in Cell Lines. The presence of mRNA corresponding to the 166 selected genes in normal tissue was evaluated using the cDNA panel derived from normal tissues (see Materials and Methods). Successful RT-PCR amplifications were achieved for 144 of the 166 genes, of which 41 exhibited expression in the majority of tissues tested, 32 exhibited selective expression but were in three or more somatic tissues and 71 exhibited expression only in testis, ovary, and/or placenta (41 of 71), or in these tissues and no more than two other somatic tissues (30 of 71).

The expression of the 71 genes with testis-predominant expression was evaluated by is RT-PCR in 21 cancer cell lines: seven derived from melanoma (SK-MEL-10, -24, -37, -49, -55, -80, -128), four from small cell lung cancer (NCI-H82, -H128, -H187, -1-1740), three from non-small cell lung cancer (SK-LC-5, -14, -17), three from colon cancer (SW403, SW480, LS174T), one from renal cancer (SK-RCC-1), one from hepatocellular carcinoma (SK-HEP-1), one from bladder cancer (T24), and one from sarcoma (SW982). Each of these cell lines expresses at least one known CT gene (data not shown).

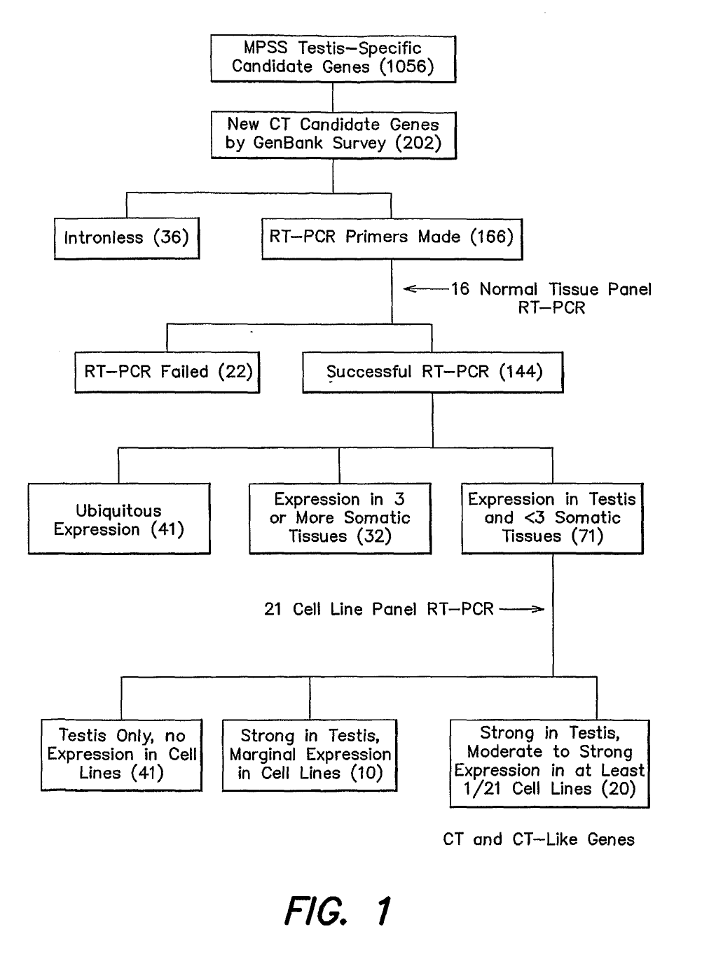

The 71 genes fell into three groups, based on their expression in the cancer cell lines used. Forty-one genes exhibited no detectable expression in any of the cell lines, 10 exhibited only very low level expression (relative to expression levels in testis), and 20 exhibited moderate to strong expression in at least one cell line. The entire screening process is summarized in FIG. 1. Table 2 lists the final group of 20 CT and CT-like genes and their expression in normal tissues and Table 3 shows their expression in the 21 cell lines.

TABLE 2

| # | Ensembl ID | Gene Name | UniGene# | Acc. No. | Chr. | Expression in normal tissues by RT-PCR |
|---|---|---|---|---|---|---|
| 1 | ENSG00000105549 | THEG | Hs.250002 | NM_016585 | 19 | Testis only, strong expression, 2 alt. spliced forms |
| 2 | ENSG00000117148 | LOC81569 | Hs.2149 | NM_030812 | 1 | Strong in testis, weak in placenta |
| 3 | ENSG00000187262 | MGC27005 | Hs.460933 | NM_152582 | X | Testies only, strong expression, 3 alt. spiced forms |
| 4 | ENSG00000160505 | NALP4 | Hs.351637 | NM_134444 | 19 | Strong in testis and ovary, weak in pancreas |
| 5 | ENSG00000133247 | COXVIB2 | Hs.329540 | NM_144613 | 19 | Strong in testis, weak in thymus, heart |
| 6 | N.A. | LOC348120 | Hs.116287 | BC047459 | 15 | Testis only, 2 alt. spliced forms |
| 7 | ENSG00000140481 | FLJ32855 | Hs.383206 | NM_182791 | 15 | Strong in testis, lung, moderate in placenta, weak in ovary |
| 8 | ENSESTG00000023728 | LOC196993 | Hs.97823 | BC048128 | 15 | Testis only, strong expression |
| 9 | ENSG00000166049 | LOC139135 | Hs.160594 | NM_173493 | X | Testis only, strong expression |
| 10 | N.A. | IMAGE164099 | Hs.408584 | BX103208 | 3 | Testis only, strong expression |
| 11 | ENSG00000104804 | TULP2 | Hs.104636 | NM_003323 | 19 | Testis only, strong expression |
| 12 | ENSESTG00000013526 | IMAGE1471044 | Hs.362492 | AA884595 | 7 | Testis only, strong expression |
| 13 | ENSESTG00000024371 | FLJ25339 | Hs.411239 | BC057843 | 16 | Testis only, strong expression, 2 alt. spliced forms |
| 14 | ENSG00000151962 | MGC271016 | Hs.133095 | NM_144979 | 4 | Testis only, strong expression |
| 15 | N.A. | IMAGE4837072 | Hs.371922 | BC040308 | 6 | Testis only, strong expression |
| 16 | N.A. | IMAGE5173800 | Hs.121221 | BI818097 | 9 | Strong in testis, weak in pancreas |
| 17 | ENSG00000101448 | SPINLW1 | Hs.121084 | NM_181502 | 20 | Testis only, strong expression |
| 18 | ENSG00000178093 | SSTK | Hs.367871 | NM_032037 | 19 | Strong in testis, weak (+/−) in multiple tissues |
| 19 | ENSG00000168594 | ADAM29 | Hs.126836 | NM_014269 | 4 | Testis only, strong expression |
| 20 | ENSG00000173421 | LOC339834 | Hs.383008 | NM_178173 | 3 | Testis only, strong expression |

TABLE 3

| | | LOC- | MGC27005/ | | | | | | | | |
|---|---|---|---|---|---|---|---|---|---|---|---|
| | THEG | 87569 | CT45 | NALP4 | COXVIB2 | LOC348120 | FLJ32855 | LOC196993 | LOC139135 | IMAGE164099 | TULP2 |
| SK-Mel-10 | ++ | +++ | ++ | + | + | − | − | − | ++ | + | + |
| SK-Mel-24 | +++ | + | + | + | − | + | − | − | − | + | + |
| SK-Mel-37 | ++ | +++ | +++ | +++ | − | +++ | − | + | +++ | − | + |
| SK-Mel-49 | − | ++ | ++ | +++ | − | ++ | + | − | − | − | + |
| SK-Mel-55 | ++ | ++ | +++ | ++ | − | − | + | − | − | + | + |
| SK-Mel-80 | + | + | − | − | ++ | − | +++ | + | − | − | + |
| SK-Mel-128 | ++ | ++ | ++ | − | +++ | +++ | − | + | − | − | + |
| NCI-H82 | +++ | +++ | + | + | + | − | ++ | + | − | +++ | + |
| NCI-H128 | +++ | − | − | − | ++ | − | +++ | + | − | − | + |
| NCI-H187 | +++ | + | − | +++ | − | − | +++ | − | − | − | +++ |
| NCI-H740 | ++ | − | − | − | + | − | +++ | ++ | − | − | − |
| SK-LC-5 | +++ | + | + | +++ | + | +++ | + | ++ | − | ++ | + |
| SK-LC-14 | ++ | ++ | +++ | + | + | − | + | − | − | ++ | + |
| SK-LC-17 | − | ++ | ++ | − | − | − | − | − | ++ | − | − |
| HCT15 | +++ | ++ | ++ | ++ | + | − | − | + | − | − | ++ |
| LS174T | − | ++ | − | +++ | ++ | +++ | − | − | − | − | − |
| SW403 | + | +++ | − | + | + | + | − | − | − | − | + |
| SW982 | ++ | − | + | − | − | − | − | + | − | + | − |
| SK-Hep-1 | − | − | − | − | ++ | − | − | − | − | − | − |
| SK-RCC-1 | ++ | + | − | − | − | − | − | − | − | − | − |
| T24 | ++ | − | + | − | − | − | − | ++ | − | − | − |
| Testis | +++ | +++ | +++ | +++ | +++ | +++ | +++ | +++ | +++ | +++ | +++ |
| ++-+++ | 15 | 11 | 8 | 7 | 5 | 5 | 5 | 3 | 3 | 3 | 2 |
| + | 2 | 5 | 5 | 5 | 7 | 2 | 4 | 7 | 0 | 4 | 12 |
| − | 4 | 5 | 8 | 9 | 9 | 14 | 12 | 11 | 18 | 14 | 7 |

| | IMAGE1471044 | FLJ25339 | MGC271016 | IMAGE4837072 | IMAGE5173800 | SPINLW1 | SSTK | ADAM29 | LOC339834 |
|---|---|---|---|---|---|---|---|---|---|
| SK-Mel-10 | + | − | − | + | − | − | − | − | + |
| SK-Mel-24 | + | − | + | − | + | − | − | − | − |
| SK-Mel-37 | − | − | − | ++ | + | + | − | − | ++ |
| SK-Mel-49 | − | − | − | − | + | − | + | + | − |
| SK-Mel-55 | + | + | − | ++ | − | − | + | − | − |
| SK-Mel-80 | + | − | − | − | + | + | − | − | − |
| SK-Mel-128 | − | − | − | − | + | ++ | + | − | − |
| NCI-H82 | ++ | − | − | − | + | − | − | − | − |
| NCI-H128 | + | ++ | − | − | + | − | ++ | − | − |
| NCI-H187 | + | − | − | − | + | − | − | ++ | − |
| NCI-H740 | − | − | − | − | + | − | − | + | − |
| SK-LC-5 | − | − | − | − | + | − | − | − | − |
| SK-LC-14 | +++ | − | +++ | − | + | − | − | − | − |
| SK-LC-17 | + | − | +++ | − | + | − | − | − | − |
| HCT15 | − | ++ | − | − | ++ | − | − | − | − |
| LS174T | − | − | − | − | − | − | − | − | − |
| SW403 | − | − | − | − | − | − | − | − | − |
| SW982 | − | − | − | − | − | − | − | − | − |
| SK-Hep-1 | + | − | − | − | + | + | − | − | − |
| SK-RCC-1 | − | − | − | − | + | − | − | − | − |
| T24 | − | − | − | − | − | − | − | − | − |
| Testis | +++ | +++ | +++ | +++ | +++ | +++ | +++ | +++ | +++ |
| ++-+++ | 2 | 2 | 2 | 2 | 1 | 1 | 1 | 1 | 1 |
| + | 8 | 1 | 1 | 1 | 14 | 3 | 3 | 2 | 1 |
| _− | 11 | 18 | 18 | 18 | 10 | 17 | 17 | 18 | 19 |

Quantitative RT-PCR of selective CT genes in tumor specimens. Of the 20 CT-like genes, 7 showed expression in at least five of the 21 (˜25%) cell lines examined (Table 3). Two of these, ENSG00000117148 (LOC81569, UniGene Hs.2149; NM_{—}030812) and ENSG00000140481 (Hs.383206; FLJ32855), exhibited strong expression in the pancreas and lung, respectively, limiting their potential utility as vaccines. These two genes may indeed encode differentiation antigens of the pancreas and lung, respectively, with concurrent testicular expression. This view is strengthened by the observation that four of five cancer cell lines expressing FLJ32855 were small-cell lung cancer lines, and that 4 of 16 ESTs corresponding to this gene were derived from lung, the remaining being from testis, placenta or brain. In comparison, ESTs derived from NM_{—}030812 were found in brain and cervix in addition to testis, indicating that this gene is probably expressed in at least a few somatic tissues.

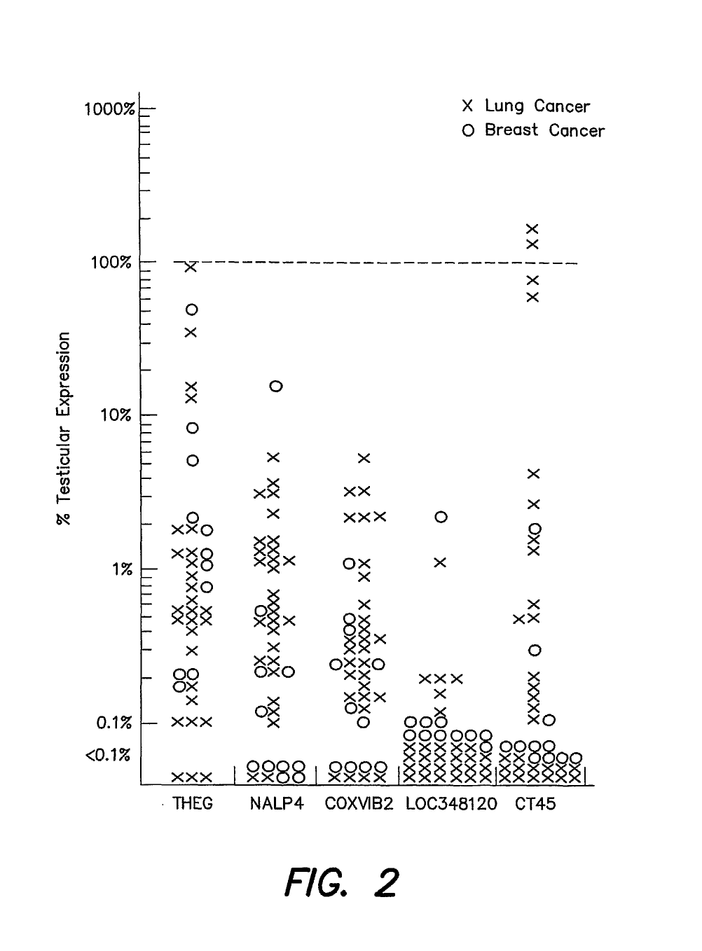

The other five genes ENSQ00000105549 (THEG), ENSG00000187262 (MGC27005), ENSG00000160505 (NALP4), ENSG00000160471 (COXVIB2), and LOC348120 (no Ensembl identifier; Unigene Hs.116287) are previously unidentified CT genes. Their expression was then measured in 29 lung tumors and 11 breast tumors by real-time RT-PCR. Table 4 lists the primer and probe sequences used. FIG. 2 shows the mRNA level distribution of these 5 genes in these specimens, expressed as percentages relative to testicular expression of these genes.

TABLE 4
Primer and probe sequences for quantitative
RT-PCR of CT genes

| | | |
|---|---|---|
| THEG | Forward: | CCAAAACCCAAGCCACATGT |
| | Reverse: | GCACTTGTCCGACTGAGCTTT |
| | Probe: | Fam-CAGACCATAACCGCCCTCCTTCACTTGG-Tamra |
| NALP4 | Forward: | TTGTCACCTCTCACCCATTGATT |
| | Reverse: | CAGGATACATTCAGATACGTCAGCTT |
| | Probe: | Fam-TGAAGTCCTTGCTGGCCTTCTAACCAACA-Tamra |
| COXVIB2 | Forward: | CCGTAACTGCTACCAGAACTTCCT |
| | Reverse: | AGTGGTACACGCGGAAATAGTACTC |
| | Probe: | Fam-ACTACCACCGCTGCCTCAAGACCAGG-Tamra |
| LOC348120 | Forward: | TGGATTCCAATTCATCTGACTACAG |
| | Reverse: | CTTCCGCTTACCTCCAACTGA |
| | Probe: | Fam-CTGCAGGTGATTCATTTGCAAGGTAAGCTG-Tamra |
| CT45 | Forward: | CTCTGCCATGTCCAAAGCAA |
| | Reverse: | AAGTCATCAATCTGAGAATCCAATTG |
| | Probe: | Fam-AAGCTTATGACAGGACATGCTATTCCACCCA-Tamra |

THEG, NALP4, COXVIB2 and LOC348120. THEO is the human ortholog of mouse Theg (testicular haploid expressed gene) (Mannan, A., et al., (2000), Cytogenet. Cell Genet., 91:171-9). RT-PCR and DNA sequencing indicated that both known splice variants of 379 and 344 amino acids are expressed in testis and in cancer. This gene was expressed in 15/21 cell lines examined by qualitative RT-PCR. By real-time RT-PCR, expression was detected in 4/29 lung tumors and 1/11 breast tumors at >10% of testicular level of expression and in 9/29 and 7/11, respectively, at >1% testicular expression.

NALP4 encodes a protein of 994 residues and contains the NTPase NACHT domain found in apoptosis-associated proteins and in proteins involved in the transcriptional activation of major histocompatibility genes and leucine-rich repeats probably involved in protein-protein interactions (Tschopp, J. et al., (2003), Nat. Rev. Mol. Cell. Biol., 4:95-104). Both NALP4 and NALP7 were identified in this study as possible CT genes. NALP7, however, was found to be only expressed weakly in three cell lines. In contrast, NALP4 was expressed in 7 of the lines with moderate to strong intensity. Furthermore, 11 of 29 lung tumors and 1 of 11 breast tumor specimens expressed NALP4 at >1% of testicular expression. However, in only one breast cancer sample was expression detected at >10% of testicular expression.

COXVIB2 encodes testis-specific cytochrome c oxidase subunit VIb (Huttemann, M. et al., (2003), Mol. Reprod. Dev., 66:8-16) and was expressed in 7 of 29 lung tumors, and 1 of 11 breast tumors expressed COXVIB2 at >1% of the testicular level of expression, but in none was it expressed at >10% of testicular expression levels.

LOC348120 encodes a hypothetical protein of 117 amino acids that has no identifiable functional domains but shows significant similarity to the mouse TLR11 (toll-like receptor 11) gene. It was expressed in only 1 of 29 lung tumors, and 1 of 11 breast tumors expressed LOC348120 at >1% of the testicular level of expression, but none exhibited expression at >10% of testicular expression levels.

A Distinctive CT multigene family on Xq26. The transcript of MGC27005 (Hs. 460933, NM_{—}152582) maps to chromosome Xq26.3 and was found to be expressed in 13 of 21 cell lines tested, with 8 of 13 showing moderate to strong expression. As measured by quantitative RT-PCR, the expression level in these 8 cell lines ranged from 0.0168 to 16.2 times that in the testis. By real-time RT-PCR, 4 of 29 lung cancer (but none of the 11 breast cancer) expressed MGC27005 at >10% of the testicular level of expression, whereas 8 of 29 and 1 of 11 of the lung and breast tumor specimens, respectively, showed MGC27005 expression at levels >1% testicular expression level.

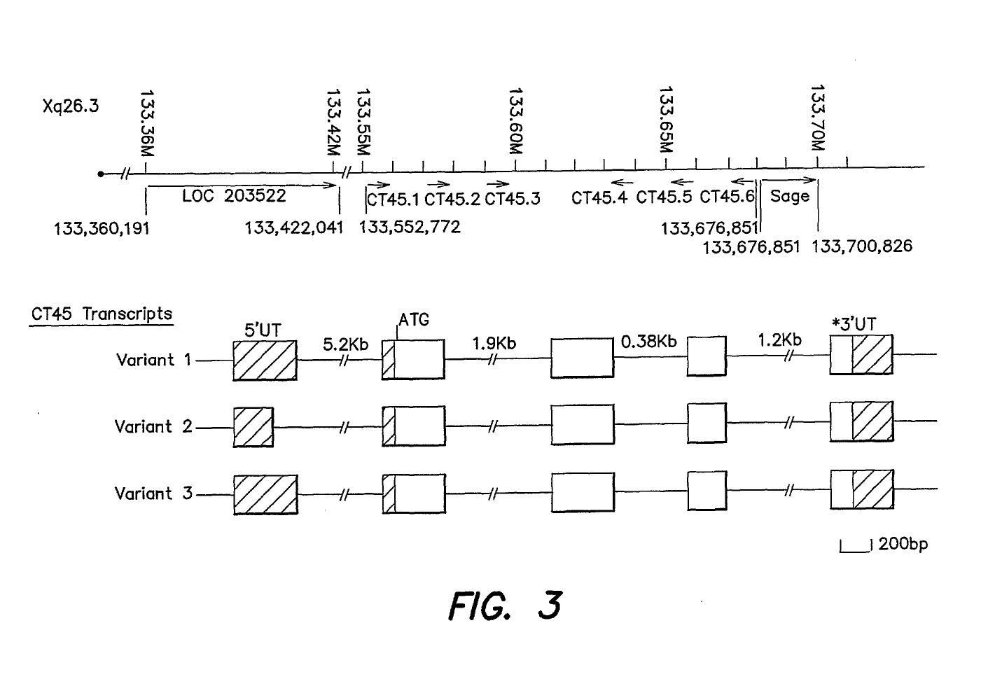

Comparison of the MGC27005 full-length sequence (GenBank Accession No. NM_{—}152582.3) to the human genome by BLASTN identified six complete copies of extremely similar genes on chromosome X (nucleotides 133550000 to 133700000 on the Ensembl genome browser), with five having previously assigned Ensembl gene entries: ENSG00000187262, ENSG00000187264, ENSG00000187265, ENSG00000187267 and ENSG00000187245. This gene family is hereby designated as CT45, following the CT nomenclature that we have proposed (Scanlan, M. J. et al., (2004), Cancer Immun., 4:1). All CT45 gene members are products of recent gene duplication events, with only 2 bp to 12 bp differences in their respective 1.0 kb transcript sequences (submitted as GenBank accession nos. AY743709 to AY743714). Thus, the CT45 transcripts detected by RT-PCR represent the accumulated expression of the CT45 gene family. Each gene spans 8-9 kb, and the genes are located in tandem within a 125 kb region (FIG. 3). The three centromeric genes are transcribed in the centromeric to telomeric direction, whereas the three telomeric genes are transcribed in the opposite direction.

An intronless copy of CT45 was identified on chromosome 5 that corresponds to the cDNA sequence of transcript variant 2 (see below), indicating that this copy on chromosome 5 is a retrogene. Although the ORF in this gene utilizes the same translational initiation site as CT45, there is a premature termination codon, resulting in a truncated 160 amino acid protein (versus 189 amino acids). This copy of CT45 on chromosome 5 is likely to be a pseudogene which may or may not be transcribed.

In addition to these complete copies, several partial gene copies were identified within the Xq26.3 region resulting from failed duplication events as has also been observed for other CT gene families, such as SSX, on chromosome X (Gure, A. O. et al., (1997), Int. J. Cancer, 72:965-71) (refer to the Cancer Immunity CT Gene Database website).

Two transcript variants of CT45 can be identified by aligning individual EST sequences against the full-length CT45 mRNA sequence (GenBank accession No. NM_{—}152582). RT-PCR analysis and DNA sequencing confirmed both transcripts in testis and in cell lines and also identified a third transcript variant (FIG. 3). All three transcripts are derived from five exons, but with exon 1 consisting entirely of a 5′ untranslated sequence varying between 85 by and 256 bp. The CT45 transcripts thus comprise a 5′ untranslated region ranging from 90 to 261 bp, a coding region of 570 bp, and a 3′ untranslated region of 292 bp, excluding the poly(A) tail. The CT45 protein consists of 189 amino acids with sequence similarity to known gene products restricted to its C-terminal 120 amino acids. Interestingly, the genes of two of the most similar proteins, LOC203522 (RefSeq NM_{—}182540) and SAGE (RefSeq NM_{—}018666), both map to Xq26 (see below).

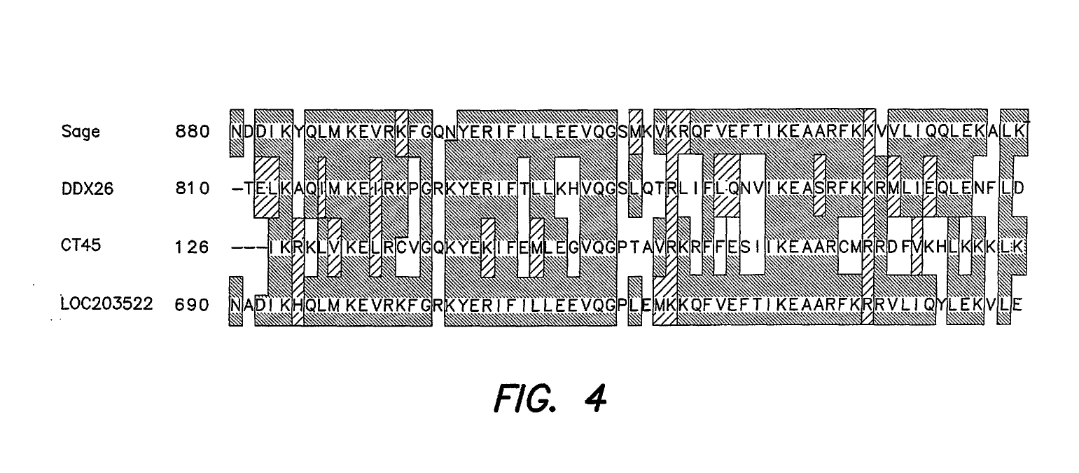

CT45 Belongs to a Distinctive Protein Family. LOC203522 is the most similar gene, with significant similarity also seen with SAGE, another CT gene (Martelange, V. et al., (2000), Cancer Res., 60:3848-55), and with DDX26 (RefSeq NM_{—}012141; SEQ ID NO:8; synonyms: DICE1, Notch12, HDB, DBI-1), a DEAD box-containing protein encoded by a gene in a region of 13q14 that has been found to be deleted in some cancers (FIG. 4). The four proteins are of different lengths. CT45 (SEQ ID NO:3) comprises 10 amino acids, DDX26 (SEQ ID NO:8) comprises 887 amino acids, and SAGE (SEQ ID NO:9) comprises 904 amino acids. LOC203522 (SEQ ID NO:7) has several putative protein products, with a 308 amino acid product (GenBank accession No. AK123209) showing homology to CT45. The observed amino acid similarity among these four proteins is restricted to their carboxyl ends. Both LOC203522 and DDX26 contain a von Willebrand factor type A domain near their N-termini that was not present in CT45.

LOC203522 is located ˜130 kb centromeric to the CT45 gene family, whereas SAGE is immediately (4.6 kb) telomeric to the CT45 genes. ESTs corresponding to LOC203522 were derived from multiple somatic tissues, and RT-PCR analysis confirmed that this gene is ubiquitously expressed in normal tissues (data not shown) whereas SAGE and CT45 are both CT genes.

Production and purification of recombinant CT45 protein. To produce recombinant CT45 protein, the full-length CT45 cDNA (corresponding to nucleotides 246-816 of RefSeq NM_{—}152582) was obtained by RT-PCR amplification from testicular RNA, cloned into BamHI and KpnI sites of pQE30 (Qiagen), and used to transform E. coli strain M15 (pREP4). The inserted CT45 cDNA was confirmed by DNA sequencing.

CT45 protein with a 5′ histidine tag derived from the pQE30 plasmid was then produced by IPTG induction of overnight culture of the transformed E. coli. Following lysis of the bacteria, CT45 protein was purified by nickel ion affinity chromatography under denaturing condition using a pH gradient. The eluted CT45, when analyzed by SDS-polyacrylamide gel electrophoresis, showed a major protein species at 31 kDa by silver staining, consistent with the predicted molecular weight.

Western blotting performed using anti-His tag antibody confirmed this major band as the recombinant CT45 protein, and this purified protein was used for immunization and monoclonal antibody production.

### CT45 Protein is Immunogenic in Cancer Patients

The immunogenicity of CT45 was tested by assaying sera from non-small cell lung cancer (NSCLC) patients for the presence of antibodies reactive with recombinant CT45 protein. The samples were tested in accordance with the protocol described in: Stocked et al., A survey of the humoral immune response of cancer patients to a panel of human tumor antigens. J Exp Med. 1998. 187(8):1349-54, and Atanackovic et al. Vaccine-induced CD4+ T cell responses to MAGE-3 protein in lung cancer patients. J Immunol. 2004. 172(5):3289-96.

Plasma samples were tested at 2 dilutions, 1/200 and 1/1000, for the presence of anti-CT45 antibodies.

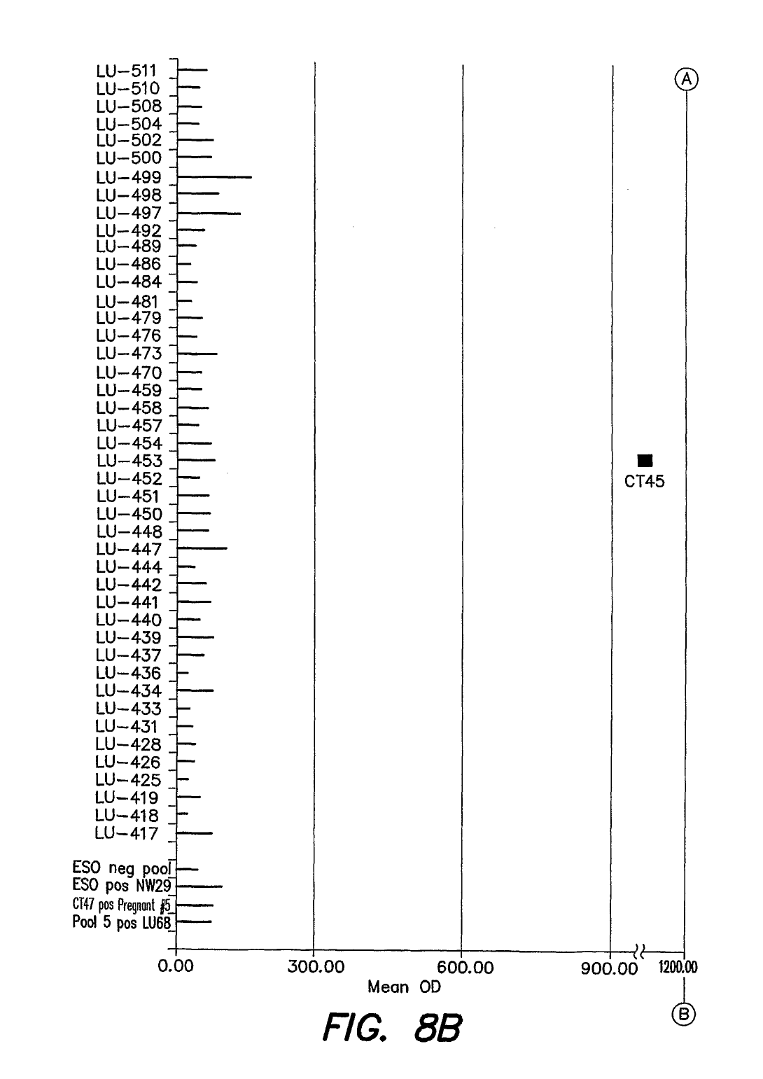

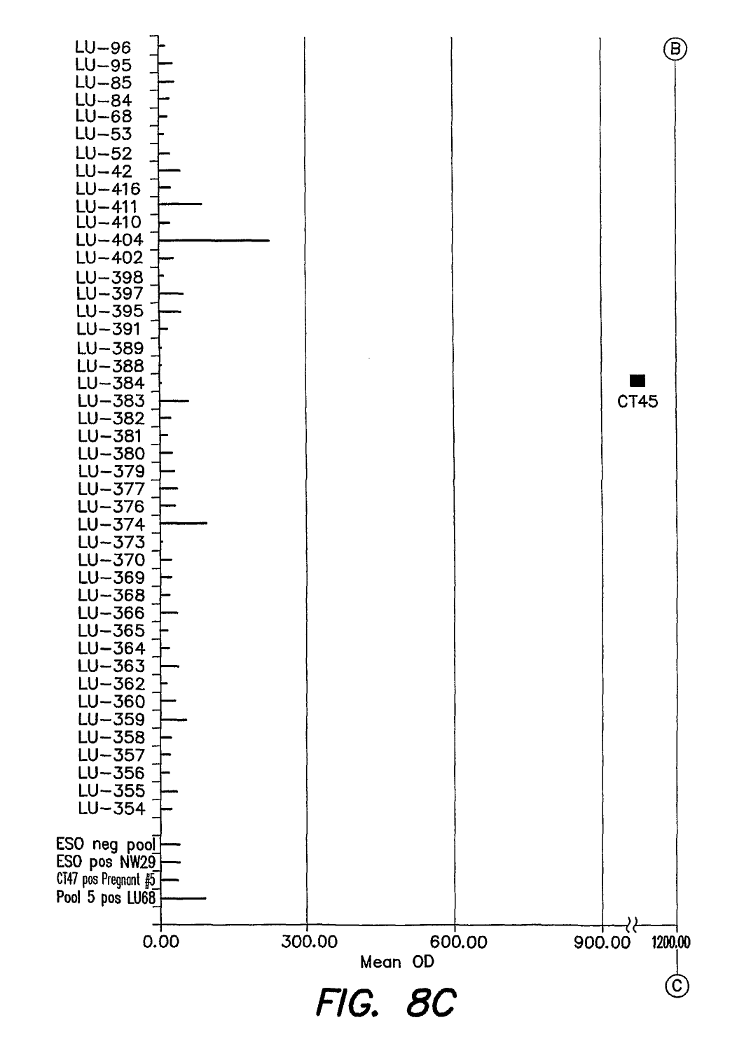

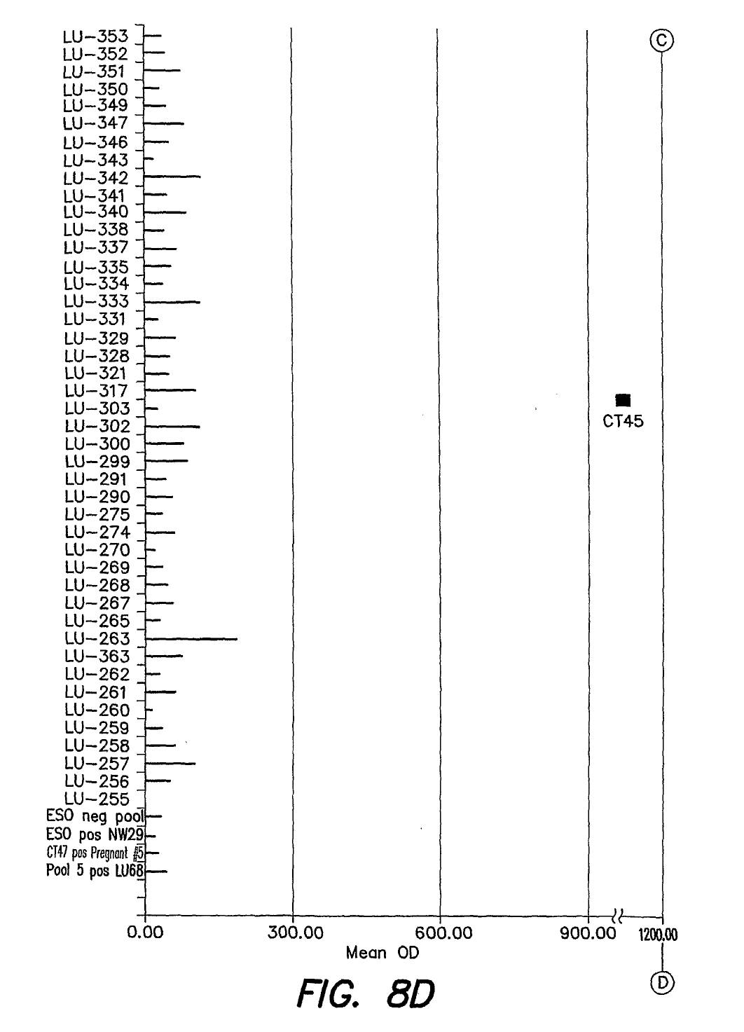

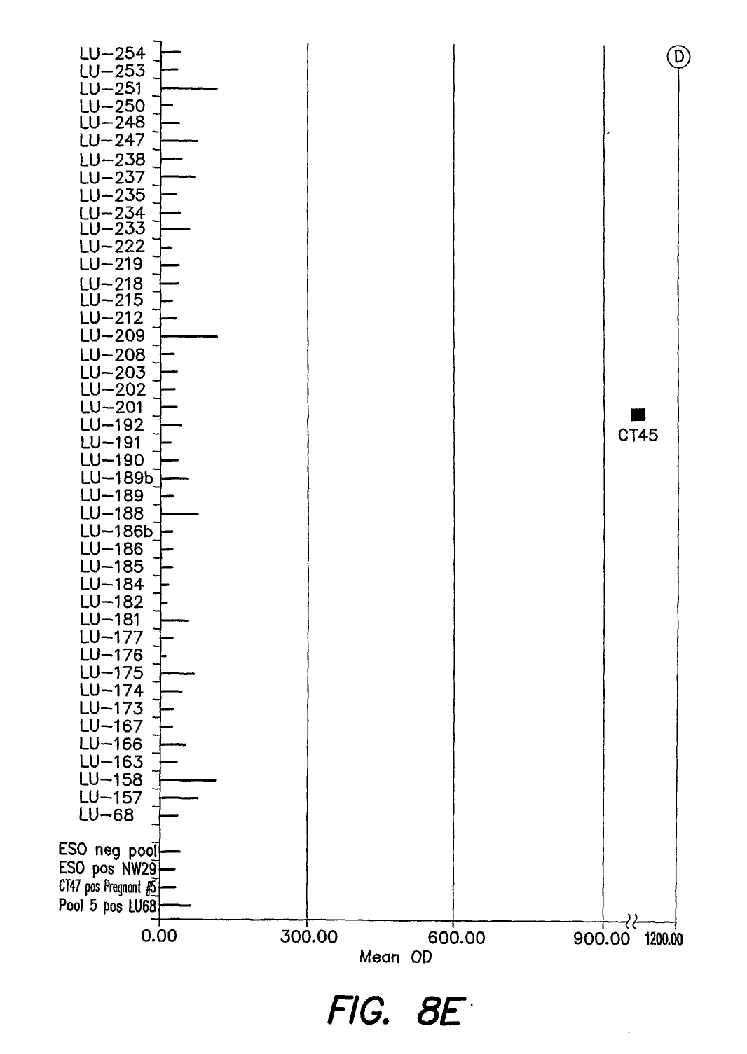

Several sera out of 175 samples tested had reactivity to CT45, as shown in FIG. 8.

### Discussion

Of 1056 genes initially identified with MPSS tags derived mainly from testis, a significant proportion were verified as being testis-specific by RT-PCR analysis. This finding illustrated that MPSS is a powerful tool for the identification of novel differentiation antigens. In this regard, MPSS should be extremely useful for identifying lineage-specific cancer vaccine targets for tumor types for which tissue-specific autoimmunity is not a major concern, such as melanoma and ovarian cancer or prostate cancer.

Our principal objective here was to identify CT genes of potential value as immunotherapeutic agents for use in human cancer. The first several CT antigens, including the MAGE, BAGE, and GAGE gene families, were all discovered on the basis of the autologous CD8+ T cell responses they elicited in cancer patients (van der Bruggen, P. et al., (2002), Immunol. Rev., 188:51-64). Subsequently a further series of CT antigen genes were identified by serological analysis of recombinant expression (SEREX) tumor cDNA libraries (Sahin, U. et al., (1995), Proc. Natl. Acad. Sci. U.S.A., 92:11810-3). The SEREX-defined CT antigens include the SSX family, SCP1, NY-ESO-1, CT7, CT8/HOM-TES-85, CAGE, CAGE1, and NY-SAR-35. More recently, CT antigens have been sought by identifying genes with restricted cancer/testis mRNA expression pattern, irrespective of their immunogenicity. This process has resulted in the identification of LAGE-1, CT9, CT10, and SAGE by representational difference analysis (Martelange, V. et al., (2000), Cancer Res., 60:3848-55; Lethe, B. et al., (1998), Int. J. Cancer, 76:903-8; Scanlan, M. J. et al., (2000), Cancer Lett., 150:155-64; Gure, A. O. et al., (2000), Int. J. Cancer, 85:726-32), and CT15, CT16, FATE, and TPTE, by EST database mining (Scanlan, M. J. et al., (2002), Int. J. Cancer, 98:485-92; Dong, X. Y. et al., (2003), Br. J. Cancer, 89:291-7). The present study, using MPSS to identify tissue specific genes with therapeutic potential, is a direct extension of the concept of identifying genes encoding CT antigens using sequence-based transcription data.

To validate the normal tissue expression, we chose to use a normalized 16 normal tissue cDNA panel from a commercial source (BD Biosciences, San Jose, Calif.) that provided standardization across this study. However, we later found it valuable to also use a second RNA source to confirm testis restriction. For example, THEG showed expression, albeit at low levels, in a few somatic tissues when tested against non-normalized cDNA synthesized from RNA of a different source (Ambion, Austin, Tex.). Such discrepancies are not uncommon in studies of this kind, and expression of CT genes should ultimately be verified by protein expression data. CT45 mRNA remains testis-restricted in both nucleic acid sources, and generation of antibody reagents against the protein product of this transcript has been undertaken as described above.

The testis-specific genes identified in this study form three groups. The first, and largest, group consists of genes that showed expression highly restricted to testis and germ cell tumors, with no evidence of expression in somatic tissue or in non-germ-cell cancers. This group of genes encodes true testis differentiation antigens, some of which are known functional proteins in germ cells, often expressed from abundant mRNAs. Examples include Protamine (PRM) 2, PRM1, and YBX2, which have 35,089, 19,397 and 5036 corresponding MPSS tags per million respectively (Steger, K. et al., (2000), Mol. Hum. Reprod., 6:219-25; Gu, W. et al., (1998), Biol. Reprod., 59:1266-74). A second group represents the true CT genes, with strong expression in a proportion of cancers. The CT45 gene family belongs to this group. The third group consists of genes that showed strong testicular expression but only marginal, low-level expression in cancer. It is clear that there is a gradient of regulation of gene expression operating in germ cells, presumably reflecting a multitude of transcriptional control mechanisms. The first group of genes is the most tightly controlled and has not yet been found to be expressed in cancers outside of germ cell lineages. The CT genes, on the other hand, are most frequently activated in cancer, probably through hypomethylation or histone deacetylation (De Smet, C. et al., (1996), Proc. Natl. Acad. Sci. U.S.A., 93:7149-53; Gure, A. O. et al., (2002), Int. J. Cancer, 101:448-53). However, even within this group, there is clearly a wide range of frequencies with which the genes are expressed in cancer, e.g. from >50% to <5% for 20 CT and CT-like genes discussed here, in the same panel of 21 cell lines. Genes in the third group are also tightly controlled, but exhibit occasional “leaky” expression in cancer. In terms of functional classification, it is debatable whether it is useful to include this third group within the CT gene category. Categorization is also complicated by the fact that some “CT genes” are expressed in selected somatic tissues. From the viewpoint of potential therapeutic utility, CT antigens that show substantial mRNA and protein expression in cancers are of most interest. Although the phenomenon of germ line gene activation and expression in tumors is of great interest and deserves full investigation, the main focus of our efforts has been on the identification of CT antigens that are truly of immunotherapeutic potential. Of the 44 CT genes/gene families in the recently created CT database (Scanlan, M. J. et al., (2004), Cancer Immun., 4:1), we estimate that probably less than a dozen would fall into this group, most of which, intriguingly, reside on the X chromosome, including MAGE, NY-ESO-1, SSX, CT7, CT10, XAGE, CAGE and SPANX. This group is now expanded by the discovery of CT45.

CT45 shares many features with other classic CT genes: a) Xq localization, which is the same as CT7 (Xq26), SAGE (Xq26), CT10 (Xq27), MAGE-A (Xq28), NY-ESO-1 (Xq28), and HOM-TES-85 (Xq24); b) multigene family, as are MAGE, GAGE, NY-ESO-1 and SSX; and c) identical or near-identical gene copies, indicating recent gene duplications, as were also described for NY-ESO-1 (Alpen, 13. et al., (2002), Gene, 297:141-9), SSX2, and SSX7 (Gure, A. O. et al., (2002), Int. J. Cancer, 101:448-53).

A protein similarity search using the CT45 sequence identified the two neighboring genes on Xq26.3, SAGE and LOC203522, as encoding proteins similar to CT45, suggesting that these three genes may be evolutionarily related. However, the exon-intron structures of these three genes are not conserved, and the gene and protein sizes are quite different. It would thus appear that, whereas these genes may be related, they have diverged significantly, so that their gene products are no longer functionally redundant. In this regard, the relationship between SAGE and CT45 is analogous to that between CT7 (MAGE-C1) and MAGE-A, two other X chromosomal CT antigen genes. The CT7 protein is 1115 amino acids long, with the N-terminus containing ten 35-amino acid tandem repeats and the carboxyl terminal sequence being non-repetitive. It is the latter region that has similarity to the other MAGE proteins, which are typically ˜310 residues in size and lack the repetitive N-terminal sequences (Chen, Y. T. et al., (1998), Proc. Natl. Acad. Sci. U.S.A., 95:6919-23). On the other hand, SAGE is a 904 amino acid protein containing thirteen 47-amino acid tandem repeats, and, again it is the carboxyl-terminal non-repetitive portion that exhibits similarity to CT45, a much smaller protein.

### Example 2

### CT46/HORMAD1 (Hs.298312, NM 032132)

In the present study, we continued our search for new CT antigens by analyzing EST database for genes with testis-predominant expression, followed by investigation of their expression in tumors by RT-PCR analysis. Of 20 CT candidate genes analyzed, we identified CT46/HORMAD1 as a novel CT antigen gene that encodes a meiosis-related protein.

### Material and Methods

### Tumor Tissues and Cell Lines.

Specimens of tumor tissues were obtained from Departments of Pathology at the Weill Medical College of Cornell University and Memorial Sloan-Kettering Cancer Center. Cell lines were obtained from the cell line bank maintained at the New York Branch of the Ludwig Institute for Cancer Research (LICR).

EST-Based Identification of Genes with a Cancer/Testis Predominant Expression Pattern.

The LICR Transcriptome database was used to search for genes showing a cancer/testis predominant expression pattern (hereafter referred to as CT-like genes). This relational database documents clusters of transcript sequences (including ESTs) aligned to the genome, and the fine structure of the genes from which they are derived (Stevenson, et al., J Infect Dis, 187 Suppl 2: S308-314, 2003). The eVOC set of controlled vocabularies (Kelso, et is al., Genome Res, 13: 1222-1230, 2003) is used to describe the origin of EST libraries contributing to the database, allowing reliable searches for genes with specific tissue expression patterns. The version of the Transcriptome DB used during this study was based on Build 30 of the NCBI assembly of the human genome.

Three pools of ESTs were derived from the database. Pool A contained ESTs derived from cDNA libraries of normal adult tissues excluding testis, ovary, placenta, pooled normal tissues, and normal tissues of unknown origin. Pool B included ESTs from libraries of any cancer types except testis. Finally, pool C contained libraries from normal testis. Normalized and subtracted libraries, as well as small libraries (less than 600 ESTs) were excluded, in an attempt to avoid non-representative EST data.

Genes showing an expression level in normal tissues (pool A) below 5% of the level observed in normal testis (pool C) but also found in cancers (pool B) were retrieved. Fisher's exact test was applied to test the significance of the representational difference observed between pools A and C for the putative CT genes, and genes with a P value <0.05 were retained. This list contained 371 candidates, among which were 7 genes already listed in the CT database (Scanlan et al. (Cancer Immun. 2004. 4:1); cancerimmunity.org/CTdatabase/): SPANXA1/CT11.1, MAGEA2/CT1.2, GAGED2/CT12.1, BORIS/CT27, HAGE/CT13, AF15q14/CT29 and TDRD1/CT41.1.

### In Silico Analysis.

To select the most promising candidates among the 371 CT genes identified, the expression profiles of each gene in normal and tumor tissues were evaluated using a combination of the SAGE Anatomic Reviewer and its Virtual Northern tool (cgap.nci.nih.gov/SAGE/AnatomicViewer), and database searches using BLASTN (ncbi.nlm.nih.gov/BLAST). The objective of the analysis was to identify Unigene clusters containing ESTs derived from testis as well as from non-germ cell tumors, but with limited expression in somatic tissues. Once a Unigene cluster was considered to be a likely CT candidate, the intron-exon structure of the corresponding gene was defined using the tools at the NCBI Web site. This information was then used to design trans-intronic primers for RT-PCR.

For specific genes of interest, e.g. CT46 (see below), various tools on the NCBI Web site were used for protein similarity searches, the identification of conserved domains, and the prediction of possible transcript variants and proteins. Gene identifiers were retrieved from the Ensembl database (ensembl.org) in order to maintain a consistent naming convention; short names were assigned to each new gene identified in the project, using Human Gene Nomenclature Committee (HGNC)-approved symbols whenever possible.

### Qualitative RT-PCR.

For RT-PCR analysis of normal tissue expression, a panel of normalized cDNA (MTC panels I and II; BD Biosciences, Palo Alto, Calif.) derived from 16 normal tissues were used. Tissues included in these panels were brain, colon, heart, kidney, leukocytes, liver, lung, ovary, pancreas, placenta, prostate, skeletal muscle, small intestine, spleen, thymus, and testis. In order to evaluate gene expression in tumor cell lines, total RNA was prepared by standard guanidinium thiocyanate-CsCl gradient method, and 2 μg was used in a 20 μl reverse transcription reaction. Two μl of the synthesized cDNA was then used per 25 μl PCR reaction. PCR were set up using a commercial master mix (Platinum Taq Supermix, Invitrogen, Carlsbad, Calif.), with 35 cycles of amplification, each consisting of 15 sec 94° C., 1 min 60° C., and 1 min 72° C. The PCR products were visualized by 1% agarose gel electrophoresis and ethidium bromide staining.

### Quantitative RT-PCR.

Quantitative RT-PCR was performed using an ABI PRISM 7000 Sequence Detection System (Applied Biosystems, Foster City, Calif.). Normal testis total RNA was obtained commercially (Ambion, Austin, Tex.). Tumor tissue total RNA was prepared using Trizol reagents (Invitrogen). Two μg total RNA was used per 20 μl reverse transcription reaction, and 2 μl cDNA was then used for each 25 μl PCR. The reactions were set up in duplicate sets, and the level of expression was determined as abundance relative to that in the testicular preparation. For this purpose, a standard curve was established for each PCR plate, consisting of testicular cDNA in 4-fold serial dilutions. Forty-five two-step cycles of amplification were performed, each cycle consisting of 15 sec at 95° C. and 1 min at 60° C. The RNA quality of the cell lines and tissues was evaluated by separate control amplification of GUS and GAPDH transcripts. All specimens included in the final analysis have Ct values differing by less than four cycles, indicating similar cDNA quality and quantity.

### Results

### Selection of CT Candidate Genes by EST-Based Database Analysis.

The LICR Transcriptome database was analyzed and transcripts with a somatic tissue EST to testicular EST ratios of <5% (statistical p-value 0.05) were selected, resulting in a list of 371 genes. Twelve of the 371 genes were already described in the literature as having a cancer/testis expression pattern, including seven listed in the recently compiled CT database (cancerimmunity.org/CTdatabase/), e.g. GAGE-D2/CT12.1, BORIS/CT27, SPANX-A1/CT11.1, MAGE-A2/CT1.2, HAGE/CT13, AF15q14/CT29 and TDRD1/CT41.1. The remaining 359 genes were manually evaluated with website bioinformatics tools to confirm the testis-specificity of the mRNA transcript and to seek evidence of expression in cancer cell lines or tissues. Two hundred and thirty genes were found to either have ESTs present in more than two somatic tissues, to have no ESTs in any cancer cDNA libraries (except germ cell tumors), or to have inadequate data available in the database. All such genes were eliminated. A sample of 20 genes was then selected from the remaining 129 genes, based on their having higher testis/normal EST ratios and the presence of ESTs from more than one type of cancer, and the mRNA distribution of these genes in normal tissues was analyzed by RT-PCR (Table 5).

TABLE 5

| | | | | Ref Seq. | | |
|---|---|---|---|---|---|---|
| LICR No. | Gene Name | Ensembl# | UniGene # | No. | Chromosome | Gene Description |
| HTR004485 | BOLL | ENSG00000152430 | Hs.169797 | NM_033030 | 2q33 | Boule-like (Drosophila) |
| HTR010472 | PRM2 | ENSG00000122304 | Hs.2324 | NM_002762 | 16q13 | Protamine 2 |
| HTR016539 | LOC440934 | N.A. | Hs.238964 | BC033986 | 2q36 | Clone IMAGE 5295746 mRNA |
| HTR022027 | LOC151273 | N.A. | Hs.244783 | BC039382 | 2q32 | Clone IMAGE 5271897 mRNA |
| HTR017116 | CPXCR1 | ENSG00000147183 | Hs.458292 | NM_033048 | Xq21 | CPX chromosome region, candidate 1 |
| HTR09806 | C10orf94 | ENSG00000171772 | Hs.117226 | NM_130784 | 10q26 | LOC93426 hypothetical gene |
| HTR07567 | HORMAD1/CT46 | ENSG00000143452 | Hs.298312 | NM_032132 | 1q21 | Hypothetical Protein DKFZp434A1315 |
| HTR016783 | FLJ33768 | ENSG00000176363 | Hs.376709 | NM_173610 | 15q22 | Hypothetical protein FLJ33768 |
| HTR015705 | PCSK4 | ENSG00000115257 | Hs.46884 | NM_017573 | 19p13 | Pro-protein convertase |
| | | | | | | sybtilisin/Kexin type 4 |
| HTR011589 | FSCN3 | ENSG00000106328 | Hs.128402 | NM_020369 | 7q31 | Fascin homolog 3, actin-binding |
| | | | | | | protein, testicular |
| HTR09020 | HCFC2 | ENSG00000111727 | Hs.55601 | NM_013320 | 12q23 | Host cell factor 2 |
| HTR005822 | MGC26979 | ENSG00000164953 | Hs.130554 | NM_153704 | 8q22 | MGC26979 hypothetical protein |
| HTR007542 | SCML2 | ENSG00000102098 | Hs.171558 | NM_006089 | Xp22 | Sex comb midleg-like 2 (Drosophila) |
| HTR005702 | DEPDC1B | ENSG00000035499 | Hs.421337 | NM_018369 | 5q12 | HbxAg transactivated protein 1 |
| HTR009187 | YBX2 | ENSG00000006047 | Hs.380691 | NM_015982 | 17p11-13 | Germ cell specific Y-box binding |
| | | | | | | protein |
| HTR009044 | NYD-SP14 | ENSG00000137473 | Hs.378893 | NM_031956 | 14q31 | NYD-SP14 protein |
| HTR006938 | NEK2 | ENSG00000117650 | Hs.153704 | NM_002497 | 1q32 | NIMA (never in mitosis gene a)- |
| | | | | | | related kinase 2 |
| HTR001543 | TP53TG3 | ENSG00000180118 | Hs.513543 | NM_015369 | 16p13 | TP53TG3 protein |
| HTR002199 | MBNL3 | ENSG00000076770 | Hs.105134 | NM_133486 | Xq26.2 | Muscleblind-like 3 (Drosophila) |
| HTR007263 | FLJ14904 | ENSG00000143194 | Hs.180191 | NM_032858 | 1q23 | Hypothetical Protein FLJ14904 |

TABLE 6

| | Tissue | | | | | | | | | | | |
|---|---|---|---|---|---|---|---|---|---|---|---|---|
| Gene | Brain | Breast | Colon | Kidney | Liver | Lung | Pancreas | Placenta | Prostate | Sk. Muscle | Spleen | Testis |
| BOLL | − | − | − | − | − | − | − | − | − | − | − | +++ |
| PRM2 | − | − | − | − | − | − | − | − | − | − | − | +++ |
| LOC440934 | − | − | − | − | − | − | − | − | − | − | − | +++ |
| LOC151273 | − | − | − | − | − | − | − | − | − | − | − | + |
| CPXCR1 | ++ | − | − | − | − | + | − | − | − | + | − | +++ |
| C10orf94 | +++ | − | − | − | − | − | − | − | − | − | − | + |
| HORMAD1/CT46 | + | + | + | − | − | − | − | + | − | − | + | +++ |
| FLJ33768 | + | + | − | ++ | − | − | − | + | − | − | − | +++ |
| PCSK4 | ++ | + | + | ++ | ++ | + | + | − | + | − | − | +++ |
| FSCN3 | ++ | ++ | ++ | ++ | + | + | + | ++ | ++ | NT | + | +++ |
| HCFC2 | ++ | ++ | ++ | ++ | ++ | ++ | ++ | ++ | ++ | ++ | ++ | ++ |
| MGC26979 | +++ | +++ | +++ | +++ | +++ | +++ | +++ | +++ | +++ | +++ | +++ | +++ |
| SCML2 | ++ | ++ | ++ | ++ | ++ | ++ | ++ | ++ | ++ | ++ | ++ | ++ |
| DEPDC1B | +++ | +++ | +++ | +++ | +++ | +++ | +++ | +++ | +++ | NT | +++ | +++ |
| YBX2 | +++ | +++ | +++ | +++ | +++ | − | +++ | − | +++ | NT | + | +++ |
| NYD-SP14 | +++ | ++ | + | +++ | − | +++ | − | + | + | NT | + | +++ |
| NEK2 | +++ | +++ | +++ | ++ | + | +++ | +++ | +++ | +++ | + | +++ | +++ |
| TP53TG3 | ++ | + | + | + | − | + | + | + | + | − | + | + |
| MBNL3 | ++ | ++ | ++ | ++ | ++ | ++ | ++ | ++ | ++ | ++ | ++ | + |
| FLJ14904 | +++ | ++ | +++ | − | + | + | ++ | +++ | +++ | + | + | +++ |

TABLE 7

| | Gene | | | | | | |
|---|---|---|---|---|---|---|---|
| | | | | | | | HORMAD1/ |
| Cell Line | BOLL | PRM2 | LOC151273 | CPXCR1 | C10orf94 | LOC440934 | CT46 |
| SK-MEL-3 | − | − | − | − | − | − | − |
| SK-MEL-10 | − | − | − | − | − | − | − |
| SK-MEL-12 | − | − | − | − | − | − | + |
| SK-MEL-14 | − | − | − | − | − | − | − |
| SK-MEL-21 | − | − | − | − | − | − | − |
| SK-MEL-24 | − | − | − | − | − | − | ++ |
| SK-MEL-28 | − | − | − | − | − | − | − |
| SK-MEL-36 | − | − | − | − | − | − | − |
| SK-MEL-37 | − | − | − | − | − | − | − |
| SK-MEL-49 | − | − | − | − | − | − | − |
| SK-MEL-55 | − | − | − | − | − | − | − |
| SK-MEL-80 | − | − | − | − | − | − | ++ |
| SK-MEL-95 | − | − | − | − | − | − | − |
| SK-MEL-108 | − | − | − | − | − | − | − |
| SK-MEL-128 | − | − | − | − | − | − | − |
| NCI-H82 | − | − | − | − | +++ | ++ | − |
| NCI-H128 | − | − | − | − | − | ++ | ++ |
| NCI-H187 | − | − | − | − | + | ++ | − |
| NCI-H740 | − | − | − | + | − | ++ | − |
| SK-LC-5 | − | − | − | − | − | + | − |
| SK-LC-14 | − | − | − | + | − | − | ++ |
| SK-LC-17 | − | − | − | − | − | − | − |
| SW403 | − | − | − | ++ | − | − | − |
| HCT-15 | − | − | − | + | − | − | − |
| LS174T | − | − | − | +++ | − | − | − |
| SK-RCC-1 | − | − | − | − | − | − | − |
| SK-HEP-1 | − | − | − | + | − | − | − |
| T24 | − | − | − | − | − | − | − |
| SW982 | − | − | − | − | − | − | − |
| testis | +++ | +++ | + | +++ | + | +++ | +++ |

### Identification of Four CT and CT-Like Genes by RT-PCR.

Ten of the selected genes showed ubiquitous expression in all 12 normal tissues examined and 3 showed differential expression, with at least moderate expression in two or more somatic tissues (Table 6). Seven genes remained as potential CT genes, including four true testis-specific genes (BOLL, PRM2, LOC440934/Hs.238964, LOC151273/Hs.244783) and 3 genes with limited and/or weak expression in somatic tissues (CPXCR1, C10orf94, formerly Hs.117226, and HORMAD1/NOHMA).

The expression of these seven genes was then evaluated in 29 cell lines, comprising 15 melanomas, four small cell lung cancers (NCI-H82, -H128, -H187, 41740), three non-small cell lung cancers (SK-LC-5, -14, -17), three colon cancers (SW403, HCT15, LS174T), one renal cancer (SK-RCC-1), one hepatocellular carcinoma (SK-HEP-1), one bladder cancer (T24), and one sarcoma (SW982). Melanoma expresses known CT antigens at a frequency higher than most other tumor types (Scanlan, et al. Cancer Immun, 4: 1, 2004). The other cell lines have been previously typed and shown to express one or more known CT genes (data not shown).

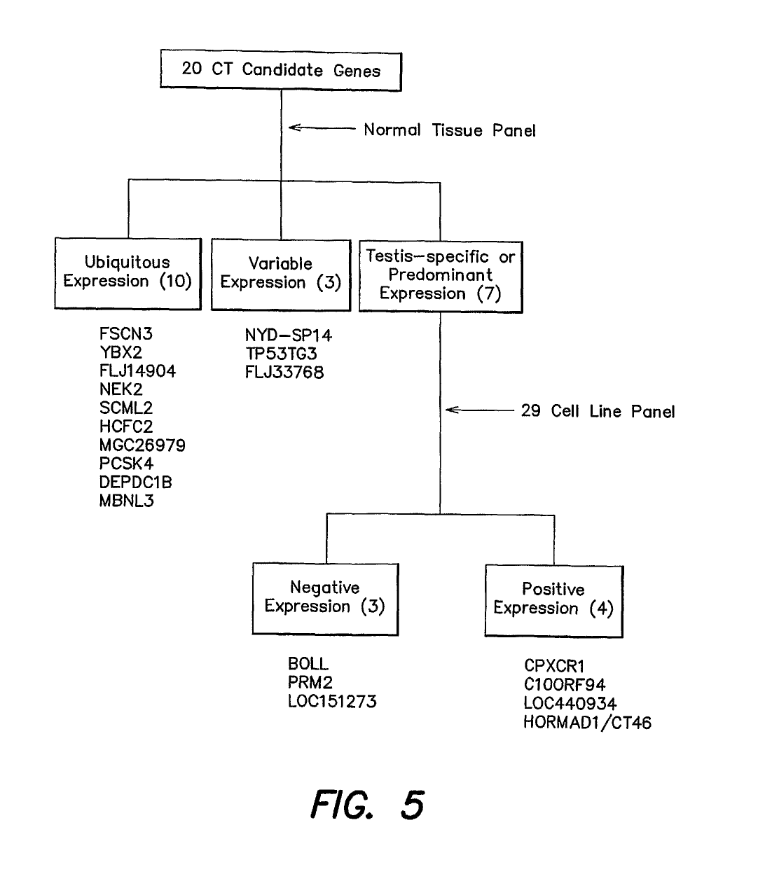

The expression profile of the seven potential CT genes in this selected “CT-rich” cell line panel is summarized in Table 7. Three genes—BOLL, PRM2, and LOC151273 (Hs.244783)—showed no expression in any of the 29 cell lines, indicating that these genes, although having cancer-derived ESTs in the GenBank, are rarely expressed in cancer. The other four genes, CPXCR1, C10orf94, LOC440934 (Hs.238964), and HORMAD1, showed at least moderate to strong expression in one or more cell lines, identifying these four genes as new CT or CT-like genes. The entire process of RT-PCR analysis of the 20 genes is summarized in FIG. 5.

Among these four genes, CPXCR1 and C10orf94 showed moderate to strong mRNA expression in normal brain by RT-PCR. LOC440934 (Hs.238964) was only expressed in five of seven cell lines derived from lung cancer (including four small cell lung cancer), but not in any of the other 22 cell lines from other cell lineages. CPXCR1, C10orf94, and LOC440934 (Hs.238964) are thus likely differentiation antigens with concurrent strong expression in testis but not in other somatic tissues, rather than true CT genes. This phenomenon has previously been observed in the case of NY-BR-1, for example, which is a breast differentiation antigen that is also expressed in testis (Jager, et al., Cancer Res, 61: 2055-2061, 2001). The products of CPXCR1 and C10orf94 are not likely to be useful as targets for cancer vaccines, as the concomitant brain expression raises the concern of anti-neuronal autoimmunity. On the other hand, LOC440934 (Hs.238964) gene product might be of value as a vaccine target for lung cancer.

In comparison to these three genes, HORMAD1 [Hs.298312, NM_{—}032132; see SEQ ID NO:25, amino acid sequence for CT46 protein (NM_{—}032132); SEQ ID NO:26, nucleotide sequence for CT46 protein (NM_{—}173493.1)] was expressed in three melanoma cell lines and two non-melanoma cell lines, and thus appeared to be a new CT gene. This gene was designated CT46, following our proposed CT nomenclature system (Scanlan, et al., Cancer Immun, 4: 1, 2004).

### Quantitative RT-PCR Analysis of CT46 Expression.

To confirm the qualitative RT-PCR data on cell lines and to evaluate further the expression of CT46/HORMAD1 in tumor tissues, quantitative RT-PCR (qRT-PCR) was performed. In addition to strong expression in testis, qualitative RT-PCR (Table 6) showed weak expression of CT46/HORMAD1 in brain, breast, colon, spleen, and placenta. This data was confirmed by qPCR. Among 11 non-testicular normal tissues, the highest expression was seen in placenta, at a level 0.76% of the testicular expression, followed by spleen (0.55%) and colon (0.23%). Other normal tissues expressed CT46/HORMAD1 mRNA at levels <0.1% of testicular expression, including breast (0.046%) and brain (0.044%).

Quantitative RT-PCR (qRT-PCR) on cell lines similarly confirmed the qualitative PCR data. Thus, of the 15 melanoma cell lines tested, the three positive lines—SK-MEL-12, -24, and -80—expressed CT46/HORMAD1 at 2.85%, 6.39%, and 8.33% of testicular expression level, respectively. All other melanoma lines, found to be negative by qualitative RT-PCR, had CT46/HORMAD1 mRNA levels that were <0.02% of the testicular expression level. There is thus 100% concordance between the qualitative and quantitative RT-PCR results. Since these two assays utilized primers derived from different regions of the genes, this data validated the expression data of CT46/HORMAD1 in normal tissue and in cell lines.

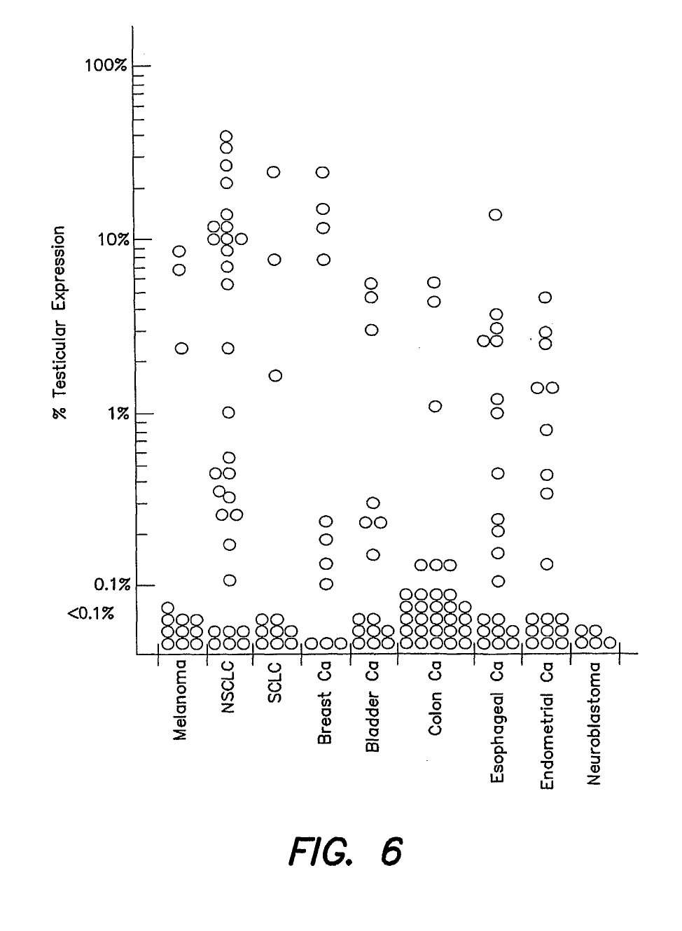

The expression of CT46/HORMAD1 in additional tumor cell lines and tumor specimens was then examined by qRT-PCR and is summarized in FIG. 6: We observed weak, moderate, and strong CT46/HORMAD1 expression by qualitative RT-PCR to be approximately equivalent to >0.1%, >1%, and >10% of testicular expression as measured by qRT-PCR. Based on these cut-off values, moderate to strong CT46/HORMAD1 expression (>1% testicular level) was seen in 14/30 (47%) non-small cell lung cancer specimens, 4/11 (36%) breast cancer specimens, 7/20 (35%) esophageal cancer specimens, 5/18 (28%) endometrial cancer specimens, 3/15 (20%) bladder cancer specimens, and 1/15 (7%) colon cancer specimens. Similar levels of expression was also seen in 4/12 (25%) small cell lung cancer cell lines and 2/17 (12%) colon cancer cell lines, but not in neuroblastoma cell lines (0/5). In total, 34 of 109 (31%) tumor specimens showed >1% testicular level of expression, with 12 of 109 (11%) exhibiting strong (>10%) expression of CT46/HORMAD1.

### CT46/HORMAD1 Protein is Immunogenic in Cancer Patients

BLAST analysis of CT46/HORMAD1 sequence against the patent database showed that a partial CT46/HORMA1 cDNA sequence had previously been identified by Obata et al. (GenBank Accession No. AX053429) by SEREX analysis of breast cancer with autologous patient serum. This indicates that CT46/HORMAD1 is immunogenic and capable of eliciting spontaneous antibody responses in cancer patients.

This has been further confirmed by testing sera from non-small cell lung cancer (NSCLC) patients for the presence of antibodies reactive with recombinant CT46/HORMAD1 protein. The samples were tested in accordance with the protocol described in: Stockert et al., A survey of the humoral immune response of cancer patients to a panel of human tumor antigens. J Exp Med. 1998. 187(8):1349-54, and Atanackovic et al. Vaccine-induced CD4+ T cell responses to MAGE-3 protein in lung cancer patients. J Immunol. 2004. 172(5):3289-96.

A total of 219 plasma samples were tested at 2 dilutions, 1/200 and 1/1000, for the presence of anti-CT46 antibodies.

Serum from a lung cancer patient (LU-68) that previously tested positive for CT-46 was used as a positive control for CT46.

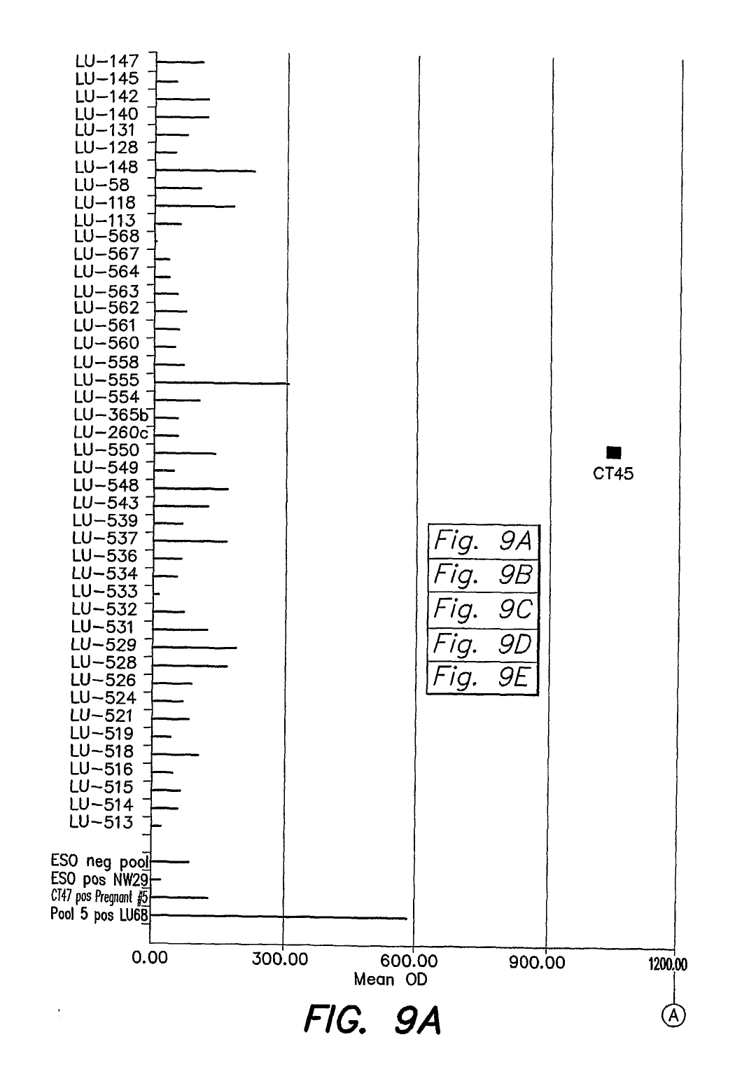

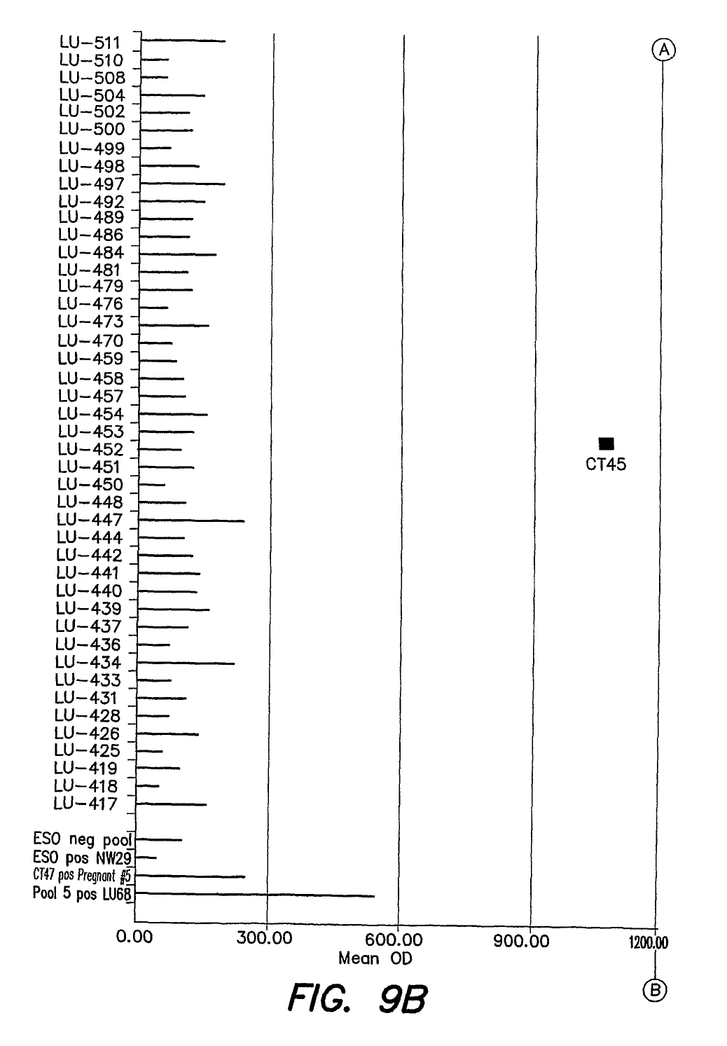

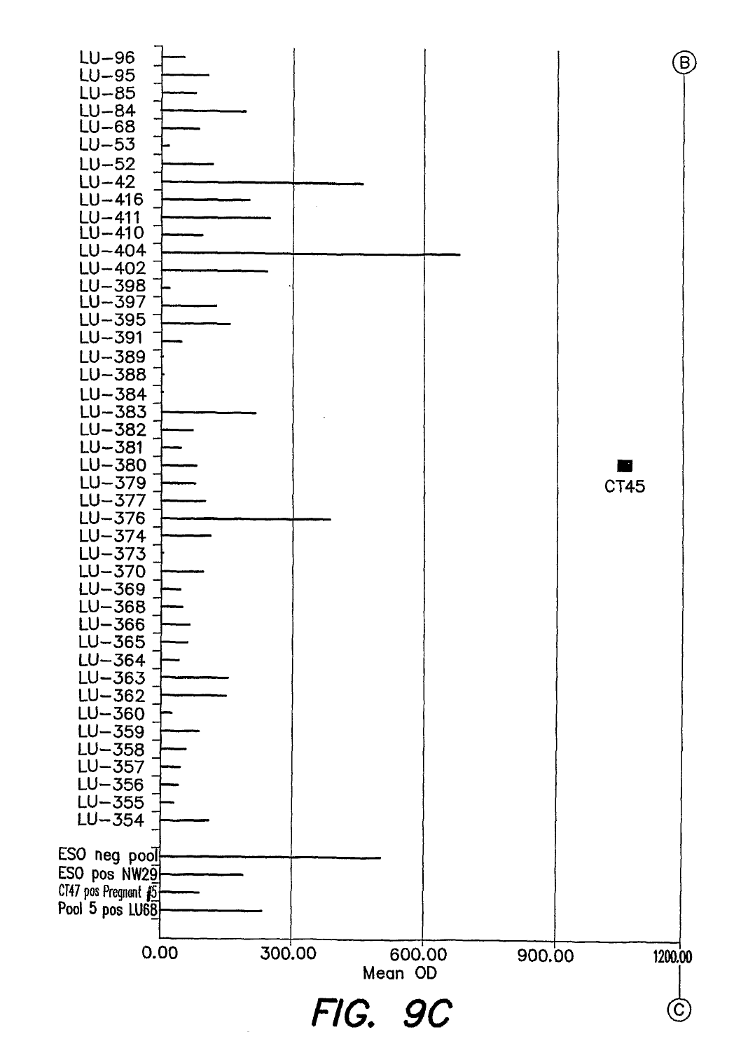

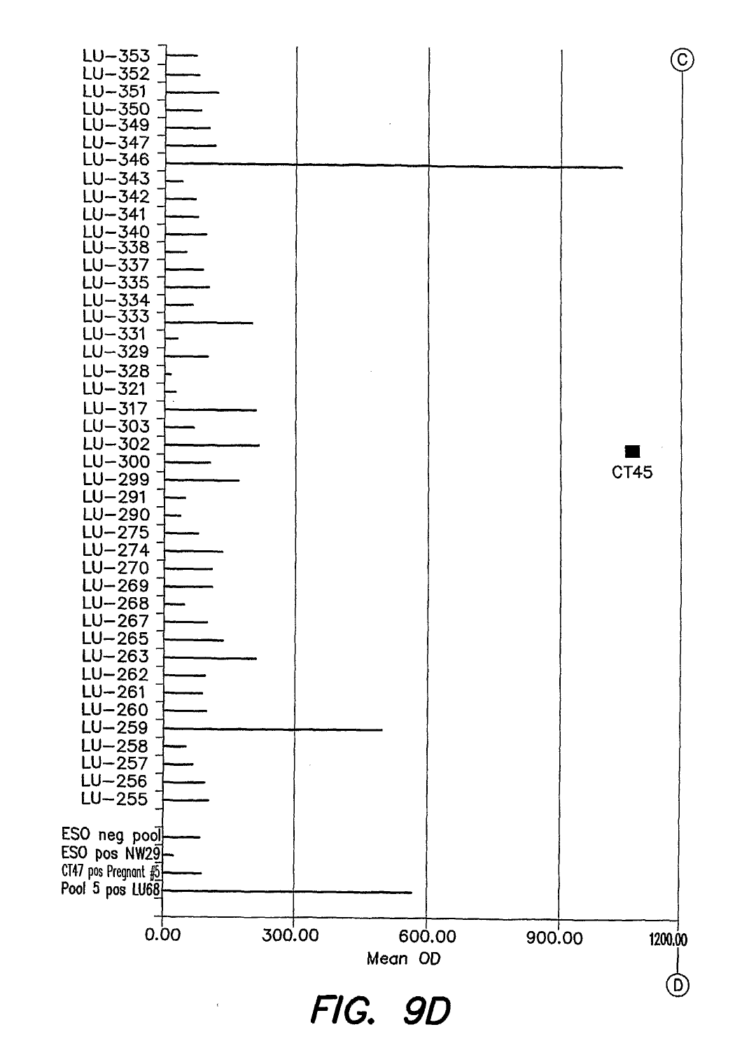

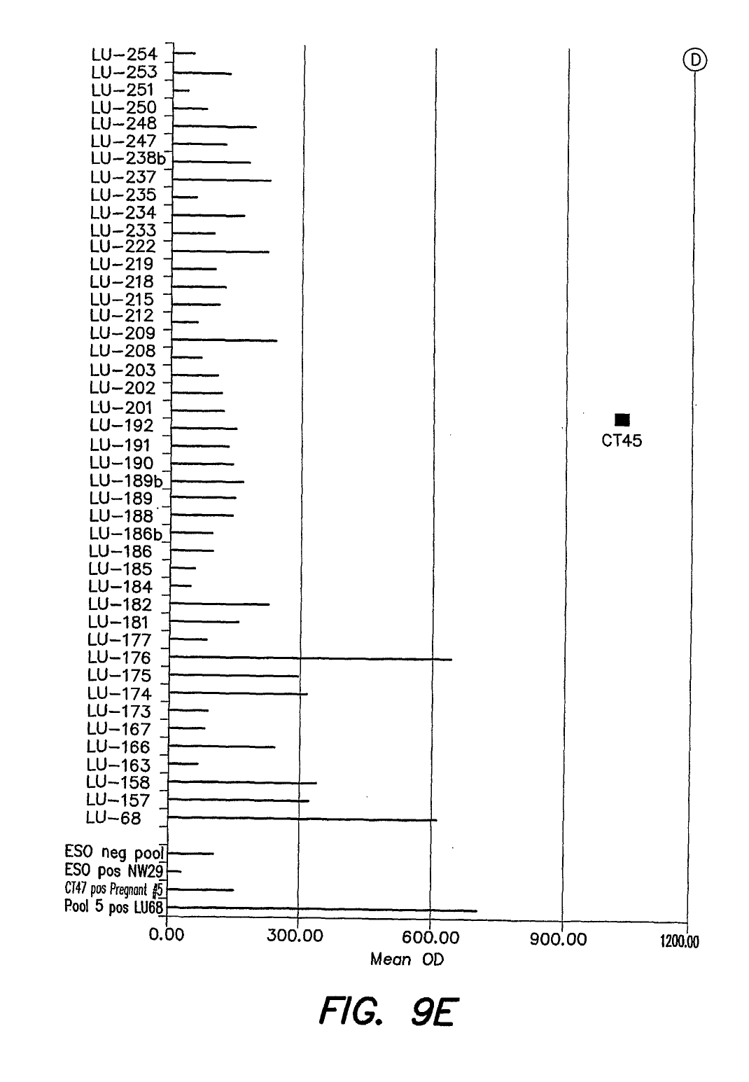

The results are shown in FIG. 9. At least six sera out of 175 had significant reactivity to CT46; additional sera were reactive with CT46 if borderline titers are included.

### The CT46/HORMAD1 Gene and Gene Products.

CT46/HORMAD1 is a single-copy gene, located on chromosome 1q21.3, that spans 22.8 kb and encodes a mRNA of 1880 bp (excluding the polyA tail). An intronless pseudogene was also identified on chromosome 6q12-14.1 (GenBank Accession No. AL132673), with 93% sequence identity to the CT46/HORMAD1 cDNA sequence.

RT-PCR and DNA sequencing of testicular CT46/HORMAD1 cDNA revealed two transcript variants. The predominant full-length CT46/HORMAD1 transcript (SEQ ID NO:26) consists of 13 exons, whereas the alternative transcript variant (SEQ ID NO:30) lacks exon 4 (64 bp). The major transcript encodes a putative protein of 394 amino acids (SEQ ID NO:25), with the translational initiation site located in exon 2. If the same initiation site is used for transcript variant 2, the encoded protein would only be 60 amino acids in length (SEQ ID NO:31), due to a frameshift in the open reading frame resulting from the missing 64 bp. Alternatively, this minor, shorter transcript may be translated from a new initiation site in exon 3, with a putative protein containing 323 amino acids (SEQ ID NO:32), of which the carboxyl 313 residues are identical to the sequences of the main product.

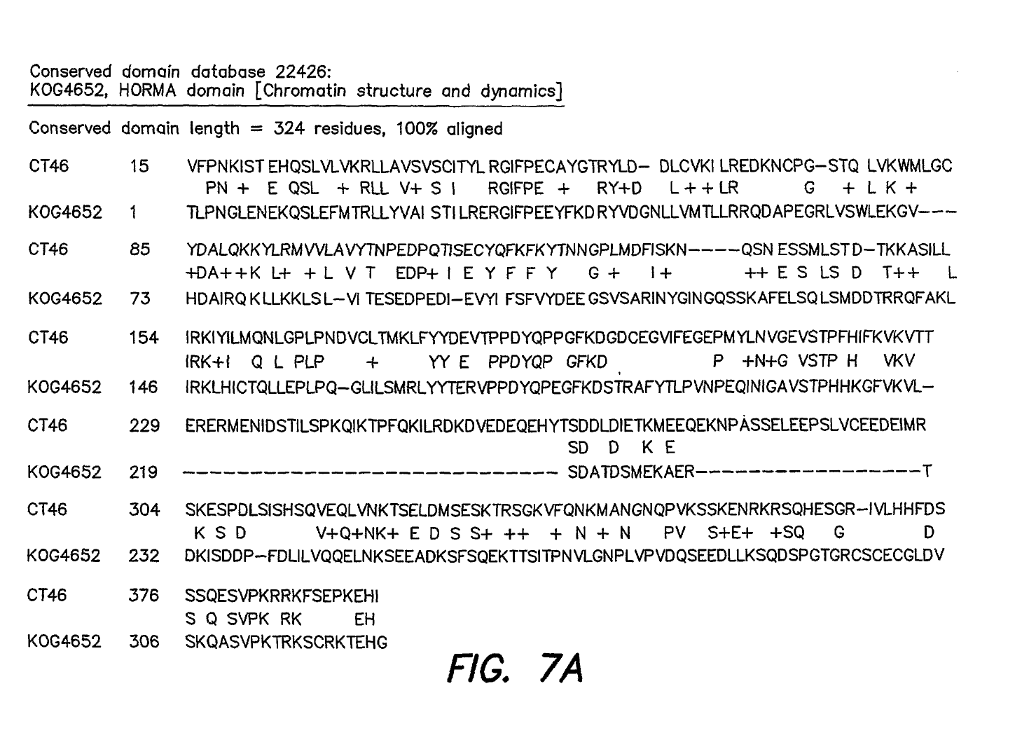

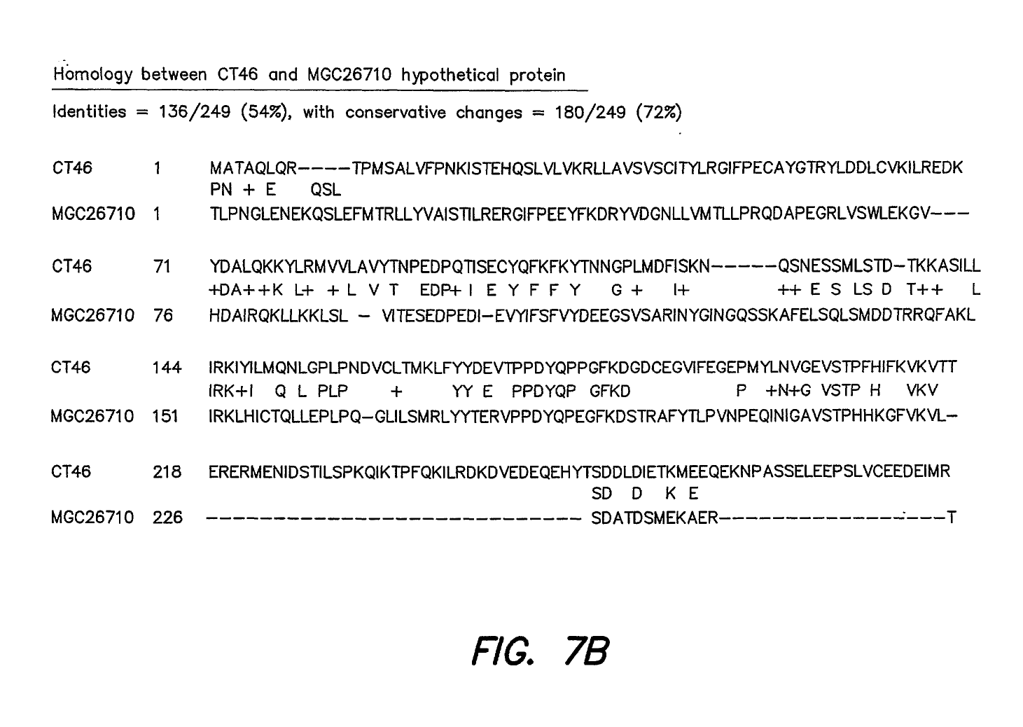

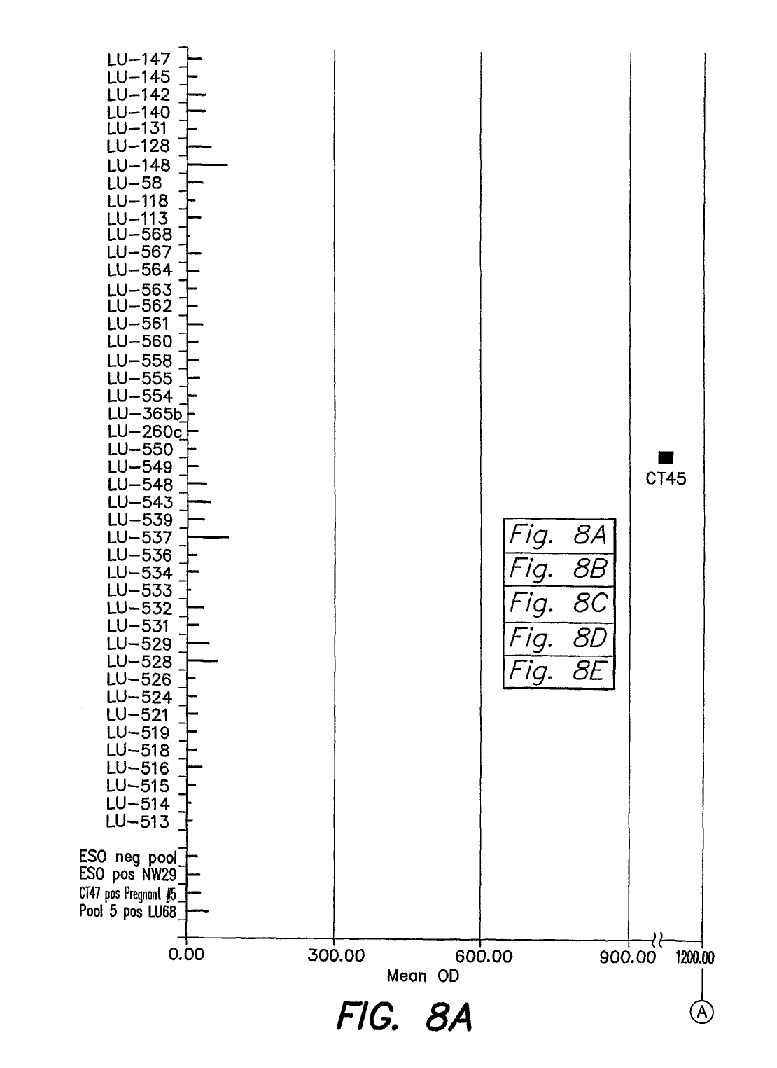

A search for conserved protein domains identified a HORMA domain comprising the entire length of the full-length 394 amino acid sequence (KOG4652, HORMA domain; and pfam02301, HORMA domain) (FIG. 7A). Indeed, while this study was ongoing, the Human Genome Organization (HUGO) named the gene HORMAD1, recognizing it as a HORMA domain-containing protein. HORMA (for Hop1p, Rev7p and MAD2) domain proteins are involved in modulating chromatin structure and dynamics. Specifically, it has been suggested that the HORMA domain recognizes chromatin states that result from DNA double strand breaks or non-attachment to the mitotic spindle and acts as an adaptor to recruit other proteins (Aravind and Koonin, Trends Biochem Sci, 23: 284-286, 1998). Hop1, the prototype HORMA domain protein, is a yeast meiosis specific protein, with which CT46/HORMAD1 shares 25.8% homology over its 215 amino acid sequence. Although it is not certain whether CT46/HORMAD1 is the human Hop1 ortholog, the presence of the HORMA domain, the similarity to Hop1 and asy1 (Arabidopsis thaliana, meiotic asynaptic mutant protein, 27.65% similarity over 260 residues), together with the germ cell-restricted expression of CT46/HORMAD1, all point to CT46/HORMAD1 being a meiosis-related protein.

### CT46/HORMAD1 is Highly Conserved Across Species.

Homology searches using predicted CT46/HORMAD1 protein sequences identified orthologs in other primates (Macaca fascicularis, GenPept Accession NO: BAB63133) as well as rodents (Mus musculus, RefSeq Accession No. NP_{—}080765; Rattus norvegicus, RefSeq Accession No. XP_{—}228333). All are hypothetical proteins predicted from cDNA sequences. Each of the cDNAs was derived from testis, indicating conserved testis-specific transcription.

The available monkey cDNA sequence (GenBank Accession No. AB070034) is a partial sequence encoding the carboxyl 298 residues, with 98.3% (293/298) sequence identity to human CT46/HORMAD1. The mouse and rat counterparts are full-length sequences, with predicted proteins of 374 amino acids and 391 amino acids, respectively. The mouse protein shows 78% sequence identity to CT46/HORMAD1 (89% similarity allowing conservative amino acid changes), and the rat protein has 72% identity to CT46/HORMAD1, with 0.83% sequence similarity including conservative changes.

In addition to identifying these ortholog genes, the protein homology search identified additional meiotic synapsis proteins, including meiotic synapsis protein from rice [GenPept Accession No. BAD00095, from Oryza sativa (japonica cultivar-group)] and the Asy1 meiotic protein from Chinese kale (GenPept Accession No. AAN37925), further supporting the hypothesis that CT46/HORMAD1 is an evolutionarily conserved meiotic protein.

### MGC26710, a Human Protein Homologous to CT46/HORMAD1.

Amongst human proteins, MGC26710 is most similar to CT46/HORMAD1. The MGC26710 gene is located on chromosome 22q12 and encodes a putative protein of 307 amino acids (RefSeq Accession No. NM_{—}152510; SEQ ID NO:33, nucleotide sequence of MGC26710; SEQ ID NO:34, amino acid sequence of MGC26710). Its similarity to CT46/HORMAD1 lies in the N-terminal HORMA domain, with 54% sequence identity in the first 240 residues, which has 72% similarity, including conservative changes (FIG. 7B).

The mRNA expression of MGC26710 in normal tissues was evaluated by qualitative RT-PCR. The results indicated tissue-restricted expression, with strong expression in testis, liver, and brain, weak expression in kidney, and no or minimal expression in eight other normal tissues. Examination of the cancer cell lines showed moderate to strong expression in 3 of 21 cell lines tested (NCI-H82, SK-LC-14 and T24), which did not coincide with CT46/HORMAD1 expression. MGC26710 is thus a differentially expressed gene, but differs from CT46/HORMAD1 in its normal and tumor tissue expression profile.

### Discussion

Through analysis of genes with predominant expression in testis we have identified CT46/HORMAD1 as a novel CT antigen. Twenty-seven ESTs from normal tissues corresponding to CT46/HORMAD1 were found in GenBank, 23 being derived from testis and four from brain tissue. By comparison, nine ESTs derived from tumor tissue were found, including four from germ cell tumors, four from breast cancer, and one from lung cancer. The EST distribution thus suggested that CT46/HORMAD1 is a germ cell-specific gene that can be activated in non-germ cell malignancies, which is characteristic of CT antigen genes. Our experimental data confirm this impression, revealing CT46 expression in lung, breast, esophageal, endometrial, bladder, and colon cancers. Although quantitative RT-PCR detected amplification products in a few somatic tissues, we could not formally exclude the possibility that this was the result of amplifying contaminating genomic DNA, as the intronless pseudogene is highly homologous, even in the region where the trans-intronic primers and probe were derived. Even if mRNA were expressed in somatic tissues, our data demonstrated that the level of expression is <1% that of testicular expression. Similar low-level expression has also been observed for other CT antigens (Scanlan, et al., Immunol Rev, 188: 22-32, 2002), which does not preclude their use as targets for cancer vaccines.

It has been observed that CT antigens can be separated into two groups, based on whether on not they are located on chromosome X. Chromosome X has been shown to contain an unusually high number of testis-specific genes (Wang, et al., Nat Genet, 27: 422-426, 2001; Warburton, et al., Genome Res, 14: 1861-1869, 2004), some of which are CT antigen genes. CT antigen genes belonging to this group include MAGE, GAGE, NY-ESO-1, SSX, XAGE, SPANX, and CT45 as described above. These genes are almost always members of multigene families, with highly similar members derived from recent gene duplication events. In contrast, most CT antigen genes not located on chromosome X are single-copy genes. CT46/HORMAD1 is a new member of the latter group.

Although the function of CT46/HORMAD1 remains to be experimentally validated, the predicted protein contains a HORMA domain, and thus is likely to be involved in regulating chromatin structure and dynamics. More specifically, CT46/HORMAD1 is highly similar to meiotic proteins, consistent with its tissue-specific expression in germ cells. This likely association with meiosis is of particular interest, as other meiosis-related proteins have also been found to be CT antigens, including Spo11 and SCP-1 (synaptoriemal complex protein 1) (Tureci, et al., Proc Natl Acad Sci USA, 95: 5211-5216, 1998). We have speculated that expression of such meiosis-specific proteins in somatic cells may lead to genome instability and thus contribute to tumor progression (Old, Cancer Immun, 1: 1, 2001).

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. A method of inducing an immune response in a subject comprising:
administering to a subject in need of such treatment an isolated polypeptide comprising an amino acid sequence set forth as SEQ ID NO:3, SEQ ID NO:4, SEQ ID NO:6, or an immunogenic fragment thereof, in an amount effective to induce an immune response in the subject,
optionally wherein the immunogenic fragment is eight or more amino acids in length;
optionally wherein the subject has or is suspected of having cancer,
preferably wherein the cancer is melanoma, small cell lung cancer, non-small cell lung cancer, colon cancer, sarcoma or bladder cancer;
optionally wherein the immune response comprises antibodies that bind to the isolated polypeptide or T cells that recognize epitopes of the isolated polypeptide presented by MHC molecules;
optionally further comprising administering an antigen presenting cell,
preferably wherein the antigen presenting cell is a dendritic cell or an autologous cell.

2.-9. (canceled)

10. A method for treating a subject comprising:
administering to a subject having or suspected of having cancer an effective amount of an antibody or antigen-binding fragment thereof that specifically binds to a CT45 polypeptide molecule that comprises an amino acid sequence as set forth in SEQ ID NO:3, or an immunogenic fragment thereof,
optionally wherein the immunogenic fragment is eight or more amino acids in length;
optionally wherein the antibody is a monoclonal antibody, a chimeric antibody, human antibody, humanized antibody, single chain antibody, (single) domain antibody or intracellular antibody; or wherein the antigen-binding fragment is a F(ab′)₂, Fab, Fd, or Fv fragment;
optionally wherein the fragment of the CT45 polypeptide molecule comprises the amino acid sequence set forth as SEQ ID NO:4 or SEQ ID NO:6.

11.-16. (canceled)

17. The method of claim 10, wherein the antibody or antigen-binding fragment thereof is bound to a cytotoxic agent.,
optionally wherein the cytotoxic agent is calicheamicin, esperamicin, methotrexate, doxorubicin, melphalan, chlorambucil, ARA-C, vindesine, mitomycin C, cisplatinum, etopside, bleomycin and/or 5-fluorouracil; or
optionally wherein the cytotoxic agent is a radioisotope,
preferably wherein the radioisotope emits α radiation, β radiation, or γ radiation, or wherein the radioisotope is ²²⁵Ac, ²¹¹At, ²¹²Bi, ²¹³Bi, ¹⁸⁶Rh, ¹⁸⁸Rh, ¹⁷⁷Lu, ⁹⁰Y, ¹³¹I, ⁶⁷Cu, ¹²⁵I, ¹²³I, ⁷⁷Br, ¹⁵³Sm, ¹⁶⁶Bo, ⁶⁴Cu, ²¹²Pb, ²²⁴Ra and/or ²²³Ra.

18.-25. (canceled)

26. A composition comprising an isolated polypeptide comprising an amino acid sequence, wherein the amino acid sequence is SEQ ID NO:3, SEQ ID NO:4, SEQ ID NO:6, or an immunogenic fragment thereof,
optionally wherein the composition comprises an amount of the isolated polypeptide effective to induce an immune response, or wherein the composition comprises an amount of the isolated polypeptide effective to induce treat cancer;
optionally further comprising a pharmaceutically acceptable carrier, and/or an antigen presenting cell, preferably wherein the antigen presenting cell is a dendritic cell or an autologous cell.

27.-32. (canceled)

33. A method of diagnosing cancer in a subject, comprising:
determining the presence or amount of a nucleic acid molecule that encodes an amino acid sequence set forth as SEQ ID NO:3 or a fragment thereof, in a biological sample isolated from the subject,
wherein the presence or amount of the nucleic acid molecule in the biological sample indicates the presence of cancer in the subject,
optionally wherein the nucleic acid molecule comprises the coding sequence of the nucleotide sequence set forth as SEQ ID NO:1, or a nucleotide sequence at least about 90% identical to the coding sequence of the nucleotide sequence set forth as SEQ ID NO:1,
preferably wherein the nucleic acid molecule comprises the coding sequence of the nucleotide sequence set forth as SEQ ID NO:1 or comprises the nucleotide sequence set forth as SEQ ID NO:1;
optionally wherein the nucleic acid molecule comprises the coding sequence of the nucleotide sequence set forth as SEQ ID NO:2, or a nucleotide sequence at least about 90% identical to the coding sequence of the nucleotide sequence set forth as SEQ ID NO:2,
preferably wherein the nucleic acid molecule comprises the coding sequence of the nucleotide sequence set forth as SEQ ID NO:2 or comprises the nucleotide sequence set forth as SEQ ID NO:2;
optionally wherein the fragment of the polypeptide sequence set forth as SEQ ID NO:3 comprises SEQ ID NO:4 or SEQ ID NO:6;
optionally wherein the presence or amount of the nucleic acid molecule in the biological sample is compared with the presence or amount of the nucleic acid molecule in a biological sample from a subject not having cancer;
optionally wherein the biological sample is tissue, cells and/or blood, and/or
optionally wherein the biological sample does not contain testis tissue.

34.-41. (canceled)

42. The method of claim 33, wherein determining the presence or amount of the nucleic acid molecule comprises contacting the biological sample with an agent that selectively binds to the nucleic acid molecule;
optionally wherein the agent that selectively binds is another nucleic acid molecule and/or
optionally wherein determining the presence or amount of the nucleic acid molecule comprises nucleic acid hybridization or nucleic acid amplification,
preferably wherein the nucleic acid amplification is PCR or wherein the nucleic acid hybridization is performed using a nucleic acid microarray, and/or
preferably wherein primers used in the method are SEQ ID NO:22 and/or SEQ ID NO:23, and/or wherein cDNA is detected.

43.-51. (canceled)

52. A method of diagnosing cancer in a subject comprising
determining the presence or amount of a CT45 polypeptide molecule comprising an amino acid sequence set forth as SEQ ID NO:3 or a fragment thereof, in a biological sample isolated from the subject,
wherein the presence or amount of the CT45 polypeptide molecule in the biological sample indicates the presence of cancer in the subject.,
optionally wherein the fragment of the polypeptide sequence set forth as SEQ ID NO:3 comprises SEQ ID NO:4 or SEQ ID NO:6;
optionally wherein the biological sample is contacted with an agent that selectively binds the CT45 polypeptide or fragment thereof,
preferably wherein the agent that selectively binds is an antibody or antigen-binding fragment thereof,
preferably wherein the antibody is a monoclonal antibody, a chimeric antibody, human antibody, humanized antibody, single chain antibody, (single) domain antibody or intracellular antibody, or wherein the antigen-binding fragment is a F(ab′)₂, Fab, Fd, or Fv fragment,
preferably wherein the antibody or antigen-binding fragment is labeled with a detectable label,
preferably wherein the detectable label is a fluorescent molecule, a radioactive molecule, an enzyme, a metal, a biotin molecule, a chemiluminescent molecule, a bioluminescent molecule, or a chromophore molecule;
optionally wherein the presence or amount of the CT45 polypeptide molecule in the biological sample is compared with the presence or amount of the CT45 polypeptide molecule in a biological sample from a subject not having cancer;
optionally wherein the biological sample is tissue, cells and/or blood, and/or
optionally wherein the biological sample does not contain testis tissue.

53.-65. (canceled)

66. A method for diagnosing cancer in a subject, comprising
determining the presence or amount of antibodies that specifically bind to a CT45 polypeptide molecule comprising an amino acid sequence set forth as SEQ ID NO:3 or a fragment thereof, in a biological sample isolated from the subject,
wherein the presence or amount of the antibodies in the biological sample indicates the presence of cancer in the subject.,
optionally wherein determining the presence or amount of antibodies comprises contacting the biological sample with CT45 polypeptide molecules comprising an amino acid sequence set forth as SEQ ID NO:3 or a fragment thereof, and determining the specific binding of the CT 45 polypeptide molecules to the antibodies,
preferably wherein the CT45 polypeptide molecules are bound to a substrate and/or wherein the CT45 polypeptide molecules comprise a detectable label, preferably wherein the detectable label is a fluorescent molecule, a radioactive molecule, an enzyme, a metal, a biotin molecule, a chemiluminescent molecule, a bioluminescent molecule, or a chromophore molecule; and/or
preferably further comprising contacting the biological sample with a detectable second antibody that binds the CT45 polypeptide molecules; and/or
preferably wherein the fragment of the CT45 polypeptide molecule comprises the amino acid sequence set forth as SEQ ID NO:4 or SEQ ID NO:6.,
optionally wherein the biological sample is tissue, cells and/or blood; and/or
optionally wherein the biological sample does not contain testis tissue.

67.-75. (canceled)

76. A method of inducing an immune response in a subject comprising:
administering to a subject in need of such treatment an isolated CT46 polypeptide molecule comprising an amino acid sequence set forth as SEQ ID NO:25, SEQ ID NO:31, SEQ ID NO:32, or an immunogenic fragment thereof, in an amount effective to induce an immune response in the subject,
optionally wherein the immunogenic fragment is eight or more amino acids in length;
optionally wherein the subject has or is suspected of having cancer,
preferably wherein the cancer is melanoma, small cell lung cancer, non-small cell lung cancer, colon cancer, bladder cancer, breast cancer, esophageal cancer, or endometrial cancer;
optionally wherein the immune response comprises antibodies that bind to the isolated polypeptide or wherein the immune response comprises T cells that recognize epitopes of the isolated polypeptide presented by MHC molecules;
optionally further comprising administering an antigen presenting cell,
preferably wherein the antigen presenting cell is a dendritic cell or an autologous cell.

77.-84. (canceled)

85. A method for treating a subject comprising:
administering to a subject having or suspected of having cancer an effective amount of an antibody or antigen-binding fragment thereof that specifically binds to a CT46 polypeptide molecule comprising an amino acid sequence set forth as SEQ ID NO:25, SEQ ID NO:31 or SEQ ID NO:32, or an immunogenic fragment thereof,
optionally wherein the immunogenic fragment is eight or more amino acids in length;
optionally wherein the antibody is a monoclonal antibody, chimeric antibody, human antibody, humanized antibody, single chain antibody, (single) domain antibody or intracellular antibody, or wherein the antigen-binding fragment is a F(ab′)2, Fab, Fd, or Fv fragment.

86.-91. (canceled)

92. The method of claim 85, wherein the antibody or antigen-binding fragment thereof is bound to a cytotoxic agent,
optionally wherein the cytotoxic agent is calicheamicin, esperamicin, methotrexate, doxorubicin, melphalan, chlorambucil, ARA-C, vindesine, mitomycin C, cisplatinum, etopside, bleomycin and/or 5-fluorouracil;
optionally wherein the cytotoxic agent is a radioisotope,
preferably wherein the radioisotope emits a radiation, β radiation or γ radiation, or
preferably wherein the radioisotope is ²²⁵Ac, ²¹¹At, ²¹²Bi, ²¹³Bi, ¹⁸⁶Rh, ¹⁸⁸Rh, ¹⁷⁷Lu, ⁹⁰Y, ¹³¹I, ⁶⁷Cu, ¹²⁵I, ¹²³I, ⁷⁷Br, ¹⁵³Sm, ¹⁶⁶Bo, ⁶⁴Cu, ²¹²Pb, ²²⁴Ra and/or ²²³Ra.

93.-98. (canceled)

99. An isolated nucleic acid molecule that encodes the amino acid sequence of SEQ ID NO:31 or SEQ ID NO:32, optionally wherein the nucleic acid molecule comprises SEQ ID NO:30.

100.-101. (canceled)

102. An isolated polypeptide comprising the amino acid sequence of SEQ ID NO:31 or SEQ ID NO:32.

103. (canceled)

104. A composition comprising an isolated polypeptide comprising an amino acid sequence, wherein the amino acid sequence is SEQ ID NO:25, SEQ ID NO:31, SEQ ID NO:32, or an immunogenic fragment thereof,
optionally wherein the composition comprises an amount of the isolated polypeptide effective to induce an immune response or to treat cancer;
optionally further comprising a pharmaceutically acceptable carrier and/or an antigen presenting cell,
preferably wherein the antigen presenting cell is a dendritic cell or an autologous cell.

105.-110. (canceled)

111. A method of diagnosing cancer in a subject, comprising:
determining the presence or amount of a nucleic acid molecule that encodes an amino acid sequence set forth as SEQ ID NO:25 or a fragment thereof, in a biological sample isolated from the subject,
wherein the presence or amount of the nucleic acid molecule in the biological sample indicates the presence of cancer in the subject,
optionally wherein the nucleic acid molecule comprises the coding sequence of the nucleotide sequence set forth as SEQ ID NO:26, or a nucleotide sequence at least about 90% identical to the coding sequence of the nucleotide sequence set forth as SEQ ID NO:26,
preferably wherein the nucleic acid molecule comprises the coding sequence of the nucleotide sequence set forth as SEQ ID NO:26 or SEQ ID NO:26,
preferably wherein the nucleic acid molecule consists of the coding sequence of the nucleotide sequence set forth as SEQ ID NO:26 or the nucleotide sequence set forth as SEQ ID NO:26;
optionally wherein determining the presence or amount of the nucleic acid molecule comprises contacting the biological sample with an agent that selectively binds to the nucleic acid molecule,
preferably wherein the agent that selectively binds is another nucleic acid molecule,
preferably wherein determining the presence or amount of the nucleic acid molecule comprises nucleic acid hybridization or nucleic acid amplification,
preferably wherein the nucleic acid amplification is PCR,
preferably wherein the nucleic acid hybridization is performed using a nucleic acid microarray
preferably wherein cDNA is detected;
optionally wherein the biological sample is tissue, cells and/or blood; and/or
optionally wherein the biological sample does not contain testis tissue;
optionally wherein the presence or amount of the nucleic acid molecule in the biological sample is compared with the presence or amount of the nucleic acid molecule in a biological sample from a subject not having cancer.

112.-125. (canceled)

126. A method of diagnosing cancer in a subject comprising
determining the presence or amount of a CT46 polypeptide molecule comprising an amino acid sequence set forth as SEQ ID NO:25, SEQ ID NO:31 or SEQ ID NO:32, or a fragment thereof, in a biological sample isolated from the subject,
wherein the presence or amount of the CT46 polypeptide molecule in the biological sample indicates the presence of cancer in the subject
optionally wherein the CT46 polypeptide molecule consists of an amino acid sequence set forth as SEQ ID NO:25, SEQ ID NO:31 or SEQ ID NO:32;
optionally wherein the biological sample is tissue, cells and/or blood; and/or
optionally wherein the biological sample does not contain testis tissue;
optionally wherein the presence or amount of the CT46 polypeptide molecule in the biological sample is compared with the presence or amount of the CT46 polypeptide molecule in a biological sample from a subject not having cancer.

127. (canceled)

128. The method of claim 126, wherein the biological sample is contacted with an agent that selectively binds the CT46 polypeptide or fragment thereof
optionally wherein the agent that selectively binds is an antibody or antigen-binding fragment thereof,
preferably wherein the antibody is a monoclonal antibody,
preferably wherein the antibody is a chimeric, human, or humanized antibody,
preferably wherein the antibody is a single chain antibody,
preferably wherein the antigen-binding fragment is a F(ab′)2, Fab, Fd, or Fv fragment, or
preferably wherein the antibody or antigen-binding fragment is labeled with a detectable label, preferably wherein the detectable label is a fluorescent molecule, a radioactive molecule, an enzyme, a metal, a biotin molecule, a chemiluminescent molecule, a bioluminescent molecule, or a chromophore molecule.

129.-138. (canceled)

139. A method for diagnosing cancer in a subject, comprising
determining the presence or amount of antibodies that specifically bind to a CT46 polypeptide molecule comprising an amino acid sequence set forth as SEQ ID NO:25, SEQ ID NO:31 or SEQ ID NO:32, or a fragment thereof, in a biological sample isolated from the subject,
wherein the presence or amount of the antibodies in the biological sample indicates the presence of cancer in the subject,
optionally wherein the biological sample is tissue, cells and/or blood; and/or
optionally wherein the biological sample does not contain testis tissue.

140. The method of claim 139, wherein determining the presence or amount of antibodies comprises contacting the biological sample with CT46 polypeptide molecule comprising an amino acid sequence set forth as SEQ ID NO:25, SEQ ID NO:31 or SEQ ID NO:32, or a fragment thereof, and determining the specific binding of the CT46 polypeptide molecules to the antibodies,
optionally wherein the CT46 polypeptide molecules are bound to a substrate;
optionally wherein the CT46 polypeptide molecules comprise a detectable label,
preferably wherein the detectable label is a fluorescent molecule, a radioactive molecule, an enzyme, a metal, a biotin molecule, a chemiluminescent molecule, a bioluminescent molecule, or a chromophore molecule; and/or
preferably further comprising contacting the biological sample with a detectable second antibody that binds the CT46 polypeptide molecules.

143.-146. (canceled)
